SEQUENCE LISTING (22 sequences)

SEQ ID NO: 1 - DNA, 16 bp, Artificial Sequence
  synthetic oligonucleotide
gccgcacgcgtttaat

SEQ ID NO: 2 - DNA, 16 bp, Artificial Sequence
  synthetic oligonucleotide
gcgatcgcttgtcgac

SEQ ID NO: 3 - DNA, 19 bp, Artificial Sequence
  synthetic oligonucleotide
ctgcggccgcacgcgttta

SEQ ID NO: 4 - DNA, 22 bp, Artificial Sequence
  synthetic oligonucleotide
gccgctatcacagatctctcga

SEQ ID NO: 5 - DNA, 16 bp, Artificial Sequence
  synthetic oligonucleotide
gcgatcgcttgtcgac

SEQ ID NO: 6 - DNA, 17 bp, Artificial Sequence
  synthetic oligonucleotide
gcatcgataccgagcgc

SEQ ID NO: 7 - DNA, 15 bp, Artificial Sequence
  synthetic oligonucleotide
gggtgtcgggtgcag

SEQ ID NO: 8 - DNA, 25 bp, Artificial Sequence
  synthetic oligonucleotide
gcatcgataccgagcgcgcgatcgc

SEQ ID NO: 9 - DNA, 22 bp, Artificial Sequence
  synthetic oligonucleotide
tcgagagatctgtgatagcggc

SEQ ID NO: 10 - DNA, 4954 bp, Artificial Sequence
  viral vector
aactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaatgtcgactgatatcgaattcctgcagcccgggctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaagaaactagtagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgcgcgatcgcaaacaaacctcgagagatctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcg

SEQ ID NO: 11 - DNA, 5700 bp, Artificial Sequence
  viral vector
aactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaatctttagagggggaaactgccttttgagttgtttatatataaagttatttaaataatgaagatcatttttttctgcctataatgtttttcttgagatgatgctttcttgaaaaaaatattttcaaaggctgaaaacaaatacataagaactcagtaaactcgggaagtgtttagcttcataatcagactgtgcagaagataggaagcagcagccggatccacagcctctgattgtcccaaatcacaggagtcatcaactgagtactccaaaaaggaaaacaagccattttcagctaaaagatatgagcataatgtgtaccataatctcacagtggctgttttagaaccaagagtgtttgtgacttaatttgaatttctcaatgcaacatttctcaaaaattccttaaacgtcatgtcatagatgatttattatgtacaaaacataactgttgagaaactccatttccttgccttctgggaggaaccttaggaaacatcagcagcaggtgcaaagtattccatagagagagggctggcataaagaacatatttattcatcagttccaaatttccctgcttctgagggcttaaaaagagggatttcttgagctgaggaaattaaaaacaaaacaaacaactatgctgaaagaggactagaaatgttctgggatattgtgaaatctagacttgaaattccttctcatttccttatgcacagattttaacacccttggtttcttcggagtagtcgactgatatcgaattcctgcagcccgggctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaagaaactagtagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgcgcgatcgcaaacaaacctcgagagatctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcg

SEQ ID NO: 12 - DNA, 5674 bp, Artificial Sequence
  viral vector
aactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaatagccaggactacacagagaaaccctgtctcaaaaaaacaaaatcaaaacaaaacaaacaaacaaaaaagctaatgactccatcatgactgtaacaaacacatcagtgcggcagtgagagcccgtctgtcagcatcagcaacagcattagtcagactgtatttgtgagcatatttgcttaggtctcttctaaatacccttcacttttctctcagagaaacccagttcatcgtattctgaaaaggagcggccgtaaaggactgatcctgtctgaagcactttggtataaaagttgcttagcagtggggcagaaaagaaaaaaagcaattaagtttatatttagtgatctatctatacacatctggagcacatttgggaaagaattcaaaagggccaattcattgcatgcctcctgctacagaacgagtgtgggagtcaagctgcgatttccacagcatcagacatttattgttgacttcaaaaagttctcccacttatgtgtaattactatcctagcaaatggctctgaaatttcagcttcttaagcataaggcagagtggtcctttaaaagtaaaataaaacgtaggccctatgagataaaattaagataaattaagaatcagttacttccaagacgaagcacttatggtgcatgccttcttatataaagcagatccttaccatgtatgtgtgctgtttgcttgccaagaccaagatgtctgtcgactgatatcgaattcctgcagcccgggctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaagaaactagtagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgcgcgatcgcaaacaaacctcgagagatctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcg

SEQ ID NO: 13 - DNA, 5674 bp, Artificial Sequence
  viral vector
aactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaatgttcagtacccagacactcccatacccttatttagaagaaataaatatcatcaagtcataatatccttgactgattaagaaagccactttgtaagtgtttattaaactgtcaagaaacttacagaatttactacatgatcgttagaataactttgagtcaggacatatttgatatgacttaatcatactccctccaaaaggaaataaggctttgtgaaggtaaattatttcttcctgggttggatatgtgtttatggagtgatcattcagctgttcccaaccttcattctgaaaaggcctcagaacacttcatgatgaatcaagctgtatcctgaatagagtaaaatgaaccacttcgtaggaactatggtgtcaccacatcagcaattcttattgaaaagtgtgcatttcttattcacatatttcaaagatggtattccagaggagtgattttctcaatgtatttttcatctacaagccttcattttaagcctaccaccgtgtgtgttttcaagacagcaattatcgttttaaaatgtgcaggtctagcttgagcttctcagcaagtttctatgccaaagaaaacaccaatcctttccatttactgagaatcaatgtttaatcctcctttttgttctcatacttattacaaatcataaagaattctgagtgtcagtttgataactagaagctccatgtaccattcctgctccttattgagtcgactgatatcgaattcctgcagcccgggctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaagaaactagtagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggcgcgccacccctgcagggaattccccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgcgcgatcgcaaacaaacctcgagagatctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcg

SEQ ID NO: 14 - DNA, 255 bp, Mus musculus
ctttagagggggaaactgccttttgagttgtttatatataaagttatttaaataatgaagatcatttttttctgcctataatgtttttcttgagatgatgctttcttgaaaaaaatattttcaaaggctgaaaacaaatacataagaactcagtaaactcgggaagtgtttagcttcataatcagactgtgcagaagataggaagcagcagccggatccacagcctctgattgtcccaaatcacaggagtcatca

SEQ ID NO: 15 - DNA, 255 bp, Mus musculus
agccaggactacacagagaaaccctgtctcaaaaaaacaaaatcaaaacaaaacaaacaaacaaaaaagctaatgactccatcatgactgtaacaaacacatcagtgcggcagtgagagcccgtctgtcagcatcagcaacagcattagtcagactgtatttgtgagcatatttgcttaggtctcttctaaatacccttcacttttctctcagagaaacccagttcatcgtattctgaaaaggagcggccgtaaa

SEQ ID NO: 16 - DNA, 255 bp, Mus musculus
gttcagtacccagacactcccatacccttatttagaagaaataaatatcatcaagtcataatatccttgactgattaagaaagccactttgtaagtgtttattaaactgtcaagaaacttacagaatttactacatgatcgttagaataactttgagtcaggacatatttgatatgacttaatcatactccctccaaaaggaaataaggctttgtgaaggtaaattatttcttcctgggttggatatgtgtttat

SEQ ID NO: 17 - DNA, 746 bp, Mus musculus
ctttagagggggaaactgccttttgagttgtttatatataaagttatttaaataatgaagatcatttttttctgcctataatgtttttcttgagatgatgctttcttgaaaaaaatattttcaaaggctgaaaacaaatacataagaactcagtaaactcgggaagtgtttagcttcataatcagactgtgcagaagataggaagcagcagccggatccacagcctctgattgtcccaaatcacaggagtcatcaactgagtactccaaaaaggaaaacaagccattttcagctaaaagatatgagcataatgtgtaccataatctcacagtggctgttttagaaccaagagtgtttgtgacttaatttgaatttctcaatgcaacatttctcaaaaattccttaaacgtcatgtcatagatgatttattatgtacaaaacataactgttgagaaactccatttccttgccttctgggaggaaccttaggaaacatcagcagcaggtgcaaagtattccatagagagagggctggcataaagaacatatttattcatcagttccaaatttccctgcttctgagggcttaaaaagagggatttcttgagctgaggaaattaaaaacaaaacaaacaactatgctgaaagaggactagaaatgttctgggatattgtgaaatctagacttgaaattccttctcatttccttatgcacagattttaacacccttggtttcttcggagta

SEQ ID NO: 18 - DNA, 720 bp, Mus musculus
agccaggactacacagagaaaccctgtctcaaaaaaacaaaatcaaaacaaaacaaacaaacaaaaaagctaatgactccatcatgactgtaacaaacacatcagtgcggcagtgagagcccgtctgtcagcatcagcaacagcattagtcagactgtatttgtgagcatatttgcttaggtctcttctaaatacccttcacttttctctcagagaaacccagttcatcgtattctgaaaaggagcggccgtaaaggactgatcctgtctgaagcactttggtataaaagttgcttagcagtggggcagaaaagaaaaaaagcaattaagtttatatttagtgatctatctatacacatctggagcacatttgggaaagaattcaaaagggccaattcattgcatgcctcctgctacagaacgagtgtgggagtcaagctgcgatttccacagcatcagacatttattgttgacttcaaaaagttctcccacttatgtgtaattactatcctagcaaatggctctgaaatttcagcttcttaagcataaggcagagtggtcctttaaaagtaaaataaaacgtaggccctatgagataaaattaagataaattaagaatcagttacttccaagacgaagcacttatggtgcatgccttcttatataaagcagatccttaccatgtatgtgtgctgtttgcttgccaagaccaagatgtct

SEQ ID NO: 19 - DNA, 720 bp, Mus musculus
gttcagtacccagacactcccatacccttatttagaagaaataaatatcatcaagtcataatatccttgactgattaagaaagccactttgtaagtgtttattaaactgtcaagaaacttacagaatttactacatgatcgttagaataactttgagtcaggacatatttgatatgacttaatcatactccctccaaaaggaaataaggctttgtgaaggtaaattatttcttcctgggttggatatgtgtttatggagtgatcattcagctgttcccaaccttcattctgaaaaggcctcagaacacttcatgatgaatcaagctgtatcctgaatagagtaaaatgaaccacttcgtaggaactatggtgtcaccacatcagcaattcttattgaaaagtgtgcatttcttattcacatatttcaaagatggtattccagaggagtgattttctcaatgtatttttcatctacaagccttcattttaagcctaccaccgtgtgtgttttcaagacagcaattatcgttttaaaatgtgcaggtctagcttgagcttctcagcaagtttctatgccaaagaaaacaccaatcctttccatttactgagaatcaatgtttaatcctcctttttgttctcatacttattacaaatcataaagaattctgagtgtcagtttgataactagaagctccatgtaccattcctgctccttattga

SEQ ID NO: 20 - DNA, 704 bp, Mus musculus
gcagaatcagataagcagaatgaatcttcattataatgtactcatatccaacagtttactgactttctgatctgagtatgaatctgagtctatttcctaaccctactaatagtcaatattattatttatttctatgtctacactggcaggcattttttccatttacaacccggtcatcctgtagcatcattctatgtatttacatatttcctggtcctcctgggacaatattctagcatagttccccaccttccttcctcagcccagctgcagactcctctcttctttctttctcagtatgattgaatacatttaaaatcaacatcatctcttccactcgcctttcctctcccatctgctgatgcccacccaatccttcttttggaatcagatttaatatgaatctttaaaatcaaaatcatcttctgtttctttgccctaatccagcagctgcagatttcttctcccggcactgttctccgtctgcagctcaccaaaatgcttcttagaaaaatatgcagttgtttttctcccctatccaaaaggctggaactttcctggcttcccaattatacaatttatgcttcttttaaggattgtgaagatgatattattagaagttgagcgaattggggctgtgtatggaaggaagggaagtttctttaagtgaatgatattgggtataaaatggcacatagggctct

SEQ ID NO: 21 - DNA, 741 bp, Mus musculus
agtagaggccacagctaagaagtgtttctctatctgcaggtgcaaagggagcgtggataaatgatttttgtaaatctacctcaatgctgtacttcaagtatttcattttcacacaatccattaagagatgaaatggaatcagtaggtcattacggtcagaagtatttaaatgatttaatatgactggagatataaatctatactgtagtccttgatttcttctattcatccgaaaacctttattattcaaaagtgctcaccaggttctgcctcatgcagaaaaataccctcaagcagaggactgttgcatattcttaccatattctcccaaacttgaatggtaagcagttgtgcatcagtaccaccacccgctgccacgggtgtgcatatggagtctcacaaataagaacataaaataatgacaggaagaaaacaaaccaaaagctaaaattaccagtggagctgattagcatatgtataagagacacttgtacagatgtgggttgctttctttagaacctaagttctcagagcagtgattcttcatcattttttgagttgtgaagtcttattatttgtttgctttttatgatcatcaccagctcctcccaaaagcatatttttaaatgggaagaaataattttatttttgaacatttgctcttatttttaccctcccaaagagggtaaaaaacgctctagaggtttgcctagttatcattaattcggaatcagcagcctc

SEQ ID NO: 22 - DNA, 7435 bp, Artificial Sequence
  viral vector
aactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaatctttagagggggaaactgccttttgagttgtttatatataaagttatttaaataatgaagatcatttttttctgcctataatgtttttcttgagatgatgctttcttgaaaaaaatattttcaaaggctgaaaacaaatacataagaactcagtaaactcgggaagtgtttagcttcataatcagactgtgcagaagataggaagcagcagccggatccacagcctctgattgtcccaaatcacaggagtcatcaactgagtactccaaaaaggaaaacaagccattttcagctaaaagatatgagcataatgtgtaccataatctcacagtggctgttttagaaccaagagtgtttgtgacttaatttgaatttctcaatgcaacatttctcaaaaattccttaaacgtcatgtcatagatgatttattatgtacaaaacataactgttgagaaactccatttccttgccttctgggaggaaccttaggaaacatcagcagcaggtgcaaagtattccatagagagagggctggcataaagaacatatttattcatcagttccaaatttccctgcttctgagggcttaaaaagagggatttcttgagctgaggaaattaaaaacaaaacaaacaactatgctgaaagaggactagaaatgttctgggatattgtgaaatctagacttgaaattccttctcatttccttatgcacagattttaacacccttggtttcttcggagtagtcgactgatatcgaattcctgcagcccgggctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagtgccaccatgaccttgcacaataacagtacaacctcgcctttgtttccaaacatcagctcctcctggatacacagcccctccgatgcagggctgcccccgggaaccgtcactcatttcggcagctacaatgtttctcgagcagctggcaatttctcctctccagacggtaccaccgatgaccctctgggaggtcataccgtctggcaagtggtcttcatcgctttcttaacgggcatcctggccttggtgaccatcatcggcaacatcctggtaattgtgtcatttaaggtcaacaagcagctgaagacggtcaacaactacttcctcttaagcctggcctgtgccgatctgattatcggggtcatttcaatgaatctgtttacgacctacatcatcatgaatcgatgggccttagggaacttggcctgtgacctctggcttgccattgactgcgtagccagcaatgcctctgttatgaatcttctggtcatcagctttgacagatacttttccatcacgaggccgctcacgtaccgagccaaacgaacaacaaagagagccggtgtgatgatcggtctggcttgggtcatctcctttgtcctttgggctcctgccatcttgttctggcaatactttgttggaaagagaactgtgcctccgggagagtgcttcattcagttcctcagtgagcccaccattacttttggcacagccatcgctggtttttatatgcctgtcaccattatgactattttatactggaggatctataaggaaactgaaaagcgtaccaaagagcttgctggcctgcaagcctctgggacagaggcagagacagaaaactttgtccaccccacgggcagttctcgaagctgcagcagttacgaacttcaacagcaaagcatgaaacgctccaacaggaggaagtatggccgctgccacttctggttcacaaccaagagctggaaacccagctccgagcagatggaccaagaccacagcagcagtgacagttggaacaacaatgatgctgctgcctccctggagaactccgcctcctccgacgaggaggacattggctccgagacgagagccatctactccatcgtgctcaagcttccgggtcacagcaccatcctcaactccaccaagttaccctcatcggacaacctgcaggtgcctgaggaggagctggggatggtggacttggagaggaaagccgacaagctgcaggcccagaagagcgtggacgatggaggcagttttccaaaaagcttctccaagcttcccatccagctagagtcagccgtggacacagctaagacttctgacgtcaactcctcagtgggtaagagcacggccactctacctctgtccttcaaggaagccactctggccaagaggtttgctctgaagaccagaagtcagatcactaagcggaaaaggatgtccctggtcaaggagaagaaagcggcccagaccctcagtgcgatcttgcttgccttcatcatcacttggaccccatacaacatcatggttctggtgaacaccttttgtgacagctgcatacccaaaaccttttggaatctgggctactggctgtgctacatcaacagcaccgtgaaccccgtgtgctatgctctgtgcaacaaaacattcagaaccactttcaagatgctgctgctgtgccagtgtgacaaaaaaaagaggcgcaagcagcagtaccagcagagacagtcggtcatttttcacaagcgcgcacccgagcaggccttgaaggatcccccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagaattccccctgcagggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgcgcgatcgcaaacaaacctcgagagatctgtgatagcggccatcaagctggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcagcttatcgataccgcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcg

(19) United States
(12) Patent Application Publication
(10) Pub. No.: US 2022/0025398 A1
(43) Pub. Date: Jan. 27, 2022

(54) A SCALABLE PLATFORM FOR THE DEVELOPMENT OF CELL-TYPE-SPECIFIC VIRUSES

(71) Applicant: PRESIDENT AND FELLOWS OF HARVARD COLLEGE, Cambridge, MA (US)

(72) Inventors: Sinisa HRVATIN, Cambridge, MA (US); Mark Aurel NAGY, Cambridge, MA (US); Michael E. GREENBERG, Cambridge, MA (US)

(73) Assignee: PRESIDENT AND FELLOWS OF HARVARD COLLEGE, Cambridge, MA (US)

(21) Appl. No.: 17/311,255

(22) PCT Filed: Dec. 5, 2019
(86) PCT No.: PCT/US2019/064616

§ 371 Date: Jun. 4, 2021

Related U.S. Application Data
(60) Provisional application No. 62/775,764, filed on Dec. 5, 2018.

(51) Int. Cl. C12N 15/86; C12N 15/10

(52) U.S. Cl. CPC C12N 15/86; C12N 2750/14143; C12N 15/1065

(57) ABSTRACT

The technology described herein is directed to adeno-associated vims (AAV) vectors comprising at least one gene regulatory element (GRE) and cells comprising said vectors. In another aspect, described herein are methods of screening for said gene regulatory elements. In another aspect, described herein are nucleic acid compositions comprising a GRE as described herein.

### CROSS-REFERENCE TO RELATED APPLICATIONS

This application claims benefit under 35 U.S.C. § 119(e) of U.S. Provisional Application No. 62/775,764 filed Dec. 5, 2018, the contents of which are incorporated herein by reference in their entirety.

### GOVERNMENT SUPPORT

This invention was made with government support under Grant Nos. MH114081, GM007753, and AG000222 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Dec. 4, 2019, is named 002806-093730WOPT-SL.txt and is 47,544 bytes in size.

### TECHNICAL FIELD

Described herein are methods and compositions related to scalable platforms for identifying properties of regulatory elements of viruses, such as those elements directing cell type specificity.

### BACKGROUND

Recombinant adeno-associated viruses (rAAVs) are emerging as a favored vehicle for delivery of gene therapy, but limiting side-effects and immune responses have been observed, likely stemming in part from viral expression in off-target cell types. Recognized strategies to restrict payload expression to the desired cell type include the modification of AAV tropism and incorporation of appropriate gene regulatory elements. However, while manipulation of tropism through capsid sequence mutagenesis and selection is an area of active investigation, systematic efforts to screen or design gene regulatory sequences capable of restricting and tailoring AAV payload expression remain largely unexplored.

The incorporation of cell-type-selective gene regulatory elements (GREs) has been employed to target viral payload expression to distinct cell types. However, given size restrictions associated with the AAV genome, it has proven challenging to identify promoter regions of sufficiently small size to preserve payload flexibility while retaining cell-type-restricted gene expression. The recent appreciation that distal enhancer elements serve as the primary determinants of tissue- and cell-type-specific gene expression can help significantly improve the specificity of viral GRE-based targeting. Moreover, the short modular nature of these elements—they are typically 200-500 base pairs (bp) in length—facilitates their inclusion in viral vectors and potentially allows for subsequent multimerization or multiplexing.

Exploiting these advances for the generation of new cell-type-specific AAVs, however, will require the development of new viral screening methods. Current approaches for viral testing are laborious, expensive, and low-throughput, typically relying on the production of individual viral vectors and the assessment of expression across a limited number of cell types by in situ hybridization or immunofluorescence. The lack of a high-throughput platform for rapid development and testing is therefore a critical bottleneck impeding the generation of cell-type-specific viral reagents.

To address these issues the Inventors developed a scalable Paralleled Enhancer Single Cell Assay (PESCA) to assess the specificity of viral vectors across the full complement of cell types present in the target tissue.

### SUMMARY

Mammalian organ systems comprise a diverse array of functionally distinct cellular populations. Understanding of how these populations of cells function in healthy and diseased individuals remains hampered by the inability to effectively and selectively target and manipulate cells in their native biological contexts. Cell-type-specific recombinant adeno-associated viruses represent a promising approach to overcome these limitations, but current methods to identify and test such viruses remain laborious, expensive, and low-throughput. Described herein is PESCA, a novel scalable single-cell RNA-sequencing-based platform for the isolation of cell-type-specific viral drivers. Applying PESCA, the Inventors generated multiple viral vectors capable of robustly and specifically targeting a rare population of GABAergic interneurons in the mouse central nervous system. This study demonstrates the utility of this readily generalizable platform for developing new cell-type-specific viral reagents, with significant implications for both basic science and future therapeutic applications.

Accordingly, described herein is an adeno-associated virus (AAV) vector, including at least one inverted terminal repeat, at least one gene regulatory element (GRE), an expression cassette, and a polyadenylation tail. In some embodiments of any of the aspects, the at least one GRE exhibits cell-type specificity. In some embodiments of any of the aspects, the at least one GRE is selected from the group consisting of: GRE12, GRE19, GRE22, GRE44, and GRE80. In some embodiments of any of the aspects, the AAV is selected from the group consisting of: bovine AAV (b-AAV), canine AAV (CAAV), mouse AAV1, caprine AAV, rat AAV, avian AAV (AAAV), AAV1, AAV2, AAV3b, AAV4, AAV5, AAV6, AAV7, AAV8, AAV9, AAV10, AAV11, AAV12, and AAV13. In some embodiments of any of the aspects, the AAV vector encodes an AAV capsid without a functional Rep protein. In some embodiments of any of the aspects, the AAV vector encodes an AAV capsid without one or more of VP1, VP2 and VP3. In some embodiments of any of the aspects, a host cell includes the aforementioned AAV vector.

Also described herein is a method of screening for adeno-associated virus (AAV) cell-type specific gene regulatory elements (GREs), including labeling a library of GREs with barcodes including a nucleic acid, wherein each of the barcodes is associated with a GRE structure, function, or both, in the library of GREs, packaging the library of labeled GREs into AAV to generate an AAV library, administering the AAV library to an organism, detecting the barcodes in one or more cell types in the organism, and identifying the GRE based on the cell type of interest and detected barcodes, thereby screening cell-type specific GREs. In some embodiments of any of the aspects, labeling the library of GREs includes amplifying GREs using polymerase chain reaction (PCR) with a primer including a vector cloning site, a barcode sequence. In some embodiments of any of the aspects, the barcode sequence is about 7-15 base pairs. In some embodiments of any of the aspects, the barcode is 10 base pairs. In some embodiments of any of the aspects, packaging the library of labeled GREs into the AAV library includes shuttling of the GRE PCR products into an AAV vector. In some embodiments of any of the aspects, detecting the barcodes in one or more cell types in the organism includes single cell RNA sequencing (sc-RNA seq) or single nucleus RNA sequencing (sn-RNA seq). In some embodiments of any of the aspects, detecting the barcodes in single cells in the organism includes single cell RNA sequencing (sc-RNA seq). In some embodiments of any of the aspects, each of the barcodes is unique to a GRE in the library of GREs. In some embodiments of any of the aspects, detecting the barcodes in one or more cell types in the organism includes enrichment of RNA transcripts. In some embodiments of any of the aspects, enrichment of RNA transcripts includes reverse transcribing RNA transcripts to generate complementary DNA (cDNA), amplifying the cDNA using second strand synthesis, and transcription of the cDNA to generate RNA intermediates. In some embodiments of any of the aspects, the RNA intermediates are amplified using PCR. In some embodiments of any of the aspects, detecting the barcodes in one or more cell types in the organism includes capturing nuclei of the one or more cell types in hydrogels including cell barcode single primers.

Further described herein is a composition, including: a nucleic acid sequence at least 10%, 20%, 30%, 40%, 50%, 60%, 70%, 80%, 85%, 90%, 95%, 96%, 97%, 98%, 99% or 100% identical to part or whole of one of sequence GRE12, GRE19, GRE22, GRE44 or GRE80.

### BRIEF DESCRIPTION OF THE DRAWINGS

FIG. 1A-1D is a series of images and schematics showing the experimental strategy and GRE selection. (FIG. 1A) Paralleled Enhancer Single Cell Assay (PESCA). A library of gene regulatory elements (GREs) is inserted upstream of a minimal promoter-driven GFP. The viral barcode sequence is inserted in the 3′UTR, and the vector packaged into AAVs. Following en masse injection of the AAV library, the specificity of the constituent GREs for various cell types in vivo is determined by single-nucleus RNA sequencing, measuring expression of the barcoded transcripts in tens of thousands of individual cells in the target tissue. Finally, bioinformatic analysis determines the most cell-type-specific barcode-associated AAV-GRE-GFP constructs. pA=polyA tail. (FIG. 1B) Area-proportional Venn diagram of the number of putative GREs identified by ATAC-Seq of purified PV, SST, and VIP interneuron chromatin. Overlapping areas indicate shared putative GREs. Non-overlapping areas represent GREs that are either unique or strongly enriched in a single cell type. (FIG. 1C) Representative ATAC-seq genome browser traces of a putative GRE enriched in SST-, PV-, or VIP-interneurons. Sequence conservation across the Placental mammalian clade is also shown. (FIG. 1D) Putative GREs (n=323,369) are plotted based on average sequence conservation (phyloP, 60 placental mammals) and SST-specificity (ratio of the average ATAC-Seq signal intensity between SST samples and non-SST samples). Dashed vertical line indicates the minimal conservation value cutoff (0.5). Light grey coloring in upper right quadrant of graph indicates the 287 most SST-specific GREs selected for PESCA screening.

FIG. 2A-2J is a series of schematics and graphs showing the PESCA screen results. (FIG. 2A) PESCA library plasmid map. ITR, inverted terminal repeats; GRE, gene regulatory element; pr, HBB minimal promoter; int, intron; GFP, green fluorescent protein; WPRE, Woodchuck Hepatitis Virus post-transcriptional regulatory element; BAR, 10-mer sequence barcode associated with each GRE; pA, polyadenylation signal. (FIG. 2B) Library complexity plotted as distribution of the abundance of the 861 barcodes and 287 GREs in the AAV library. Barcodes and GREs were binned by number of sequencing reads attributed to each barcode or GRE within the library. (FIG. 2C) Transcript count per nucleus (n=32,335 nuclei). Sequencing libraries were prepared with or without PCR-enrichment for viral transcripts. PCR enrichment resulted in a 382-fold increase in the number of recovered viral transcripts (p=0) to an average of 15.6 unique viral transcripts per nucleus. Displayed as Log 10(Count+1). (FIG. 2D) t-SNE plot of 32,335 nuclei from V1 cortex of two animals. The key denotes main cell types: Exc (Excitatory neurons), Pv (PV Interneurons), Sst (SST Interneurons), Vip (VIP interneurons), Npy (NPY Interneurons), Astro (Astrocytes), Vasc (Vascular-associated cells), Micro (Microglia), Olig (Oligodendrocytes), OPCs (Oligodendrocyte precursor cells). (FIG. 2E) Marker gene expression across cell types. The gradient denotes mean expression across all nuclei normalized to the highest mean across cell types. Size represents the fraction of nuclei in which the marker gene was detected. (FIG. 2F) Three-dimensional dot plot with each dot representing one GRE (n=287). The values on each axis represent the SST fold-enrichment calculated for each GRE based on the three barcodes paired with that GRE. Plane of correlation between the enrichment values calculated from three sets of barcodes associated with 287 GREs (r=0.53±0.03, p<2.2×10⁻¹⁶). The gradient indicates the average enrichment between the three barcodes. (FIG. 2G) Pairwise Pearson correlation between the enrichment values calculated from three sets of barcodes associated with 287 GREs for experimental data (Exp. Data, r=0.53±0.03, p<2.2×10⁻¹⁶) and after random shuffling of enrichment values (Shuffled Data, r=0±0.06). (FIG. 2H) GREs ranked by average expression specificity for SST interneurons Shading indicates the minimal and maximal specificity calculated by analyzing each of the three barcodes associated with a GRE. Also shown are the five top hits that also passed a statistical test for SST interneuron enrichment (FDR-corrected q<0.01). (FIG. 2I) Expression of the top five hits: GRE12, GRE19, GRE22, GRE44, GRE80. For each GRE, expression values are split into two animals, and, for each animal, into the three barcodes associated with that GRE. Gradient denotes mean expression across all nuclei normalized to the highest mean across cell types. Size represents the fraction of nuclei in which the marker gene was detected. (FIG. 2J) t-SNE plot of 32,335 nuclei from V1 cortex of two animals, showing expression of GRE12, GRE19, GRE22, GRE44, and GRE80. Plot is pseudocolored based on the mean GRE expression in each cell type.

FIG. 3A-3M is a series of images and graphs showing hit confirmation and electrophysiology. (FIG. 3A-3D) Fluorescent images from adult Sst-Cre; Ai14 mouse visual cortex twelve days following injection with rAAV-GRE-GFP as indicated. Scale bars 100 um. (FIG. 3E) Identification of rAAV-GRE-GFP⁺ cells that express tdTomato (SST⁺). Each dot represents a GFP⁺ cell (n=2066, 172, 1164, and 765, for AAV-[ΔGRE, GRE12, GRE22, GRE44]-GFP, respectively). Dark grey dots indicates tdTomato⁺ (SST⁺) cells. Distribution of cell frequency across tdTomato intensity is plotted on the right for each construct. (FIG. 3F) Quantification of the fraction of GFP⁺ cells that are SST⁺. Each dot represents one animal. Box plot represents mean±standard error of the mean (s.e.m). Values are 27.2±1.9%, 90.7±2.1, 72.9±4.2%, and 95.8±0.6% for AAV-[ΔGRE, GRE12, GRE22, GRE44]-GFP, respectively. (FIG. 3G) Quantification of the number of GFP⁺ SST⁻ cells normalized for area of infection. Each dot represents one animal. Box plot represents mean±standard error of the mean (s.e.m). Values are 198.0±46.0, 16.4±6.2, 56.0±17.3 and 6.1±2.1 cells/mm² for AAV-[ΔGRE, GRE12, GRE22, GRE44]-GFP, respectively. (FIG. 3H) Quantification of the fraction of GFP⁺ cells that are Pr or VIP⁺. Box plot represents mean±standard error of the mean (s.e.m). Fraction of AAV-GRE-GFP⁺ cells that are PV⁺ is 1.4±1.4%, 2.2±0.7, and 4.3±1.7% for AAV-[GRE12, GRE22, GRE44]-GFP, respectively. Similarly, the fraction of AAV-GRE-GFP⁺ cells that are VIP⁺ is 1.2±1.2%, 1.3±1.3%, and 1.7±1.0% for AAV-[GRE12, GRE22, GRE44]-GFP⁺ cells, respectively. (FIG. 3I) Distribution of the location of GFP-expressing cells as function of distance from the pia. The curves indicated represents SST⁺ cells (n=2648); the remaining line represents GFP⁺ SST⁺ cells (n=2066, 172, 1164, and 765, respectively, for AAV-[ΔGRE, GRE12, GRE22, GRE44]-GFP). Shading represents the 95% confidence interval. (FIG. 3J) Quantification of the fraction of Sst-Cre; Ai14⁺ cells within the infection area that are GFP⁺. Each dot represents one animal. Box plot represents mean±standard error of the mean (s.e.m). Values are 44.5±12.0%, 73.4±9.4%, and 35.9±6.2% for AAV-[GRE12, GRE22, GRE44]-GFP, respectively. (FIG. 3K) Representative current-clamp recordings from AAV-GRE12-Gq-tdTomato⁺ cells before and during CNO application. (FIG. 3L) Increased firing rates of AAV-GRE12-Gq-tdTomato⁺ cells evoked by depolarizing current injections upon bath application of CNO (3 animals, 6-7 cells). (FIG. 3M) Robust depolarization of AAV-GRE12-Gq-tdTomato⁺ cells upon bath application of CNO (3 animals, 6-7 cells).

FIG. 4 is a series of graphs showing the identification of conserved GREs. Left: For each of the 323,369 genomic regions that were identified by ATAC-Seq as GREs in either SST⁺, VIP⁺ or PV⁺ cells, a region of the same size was chosen exactly 100,000 bases away from the GRE. The mean sequence conservation score (phyloP, 60 placental mammals) for each of these GRE-distal regions was calculated and plotted. A vertical line at the conservation score of 0.5 indicates the 95^{th} percentile of that distribution and was chosen as a minimal conservation score needed to consider a GRE sequence as conserved. Right: The mean sequence conservation score (phyloP, 60 placental mammals) for each of the 323,369 GREs was calculated and plotted. A vertical line indicates the minimal conservation score of 0.5. 36,215 GREs (11%) had a mean conservation greater than 0.5 and were deemed conserved.

FIG. 5 is a schematic showing PESCA library construction. PCR is used to amplify GREs from the genomic DNA and to introduce appropriate restriction enzyme sites and, subsequently, a 10 bp barcode sequence. Each GRE is amplified three times using three different barcode sequences. The amplified GREs are pooled and cloned into an AAV vector. Restriction enzyme sites between the GRE and the barcode are used to insert an expression cassette consisting of a minimal promoter, intron, GFP and WPRE sequences. See e.g., experimental methods section for details.

FIG. 6A-6F shows a series of graphs. (FIG. 6A) Dot plot of the number of unique molecular identifiers (UMIs) and the number of genes for each nucleus that was analyzed. (FIG. 6B) Plot showing the density distribution of number of UMIs and genes per nucleus. (FIG. 6C) Distribution of the number of unique barcodes and unique GREs detected per nucleus, displayed as Log 10(Count+1). (FIG. 6D) Quantification of the fraction of cells within each defined cell type in which the Inventors detected barcoded viral transcripts. Each dot represents one animal. Box plot represents mean±standard error of the mean (s.e.m) for Exc (86.6±4.6%), Int_Pv (91.6±3.1%), IntSst (93.9±2.1%), Int_Vip (90.4±3.5%), Int_Npy (87.1±3.8%), Astro (82.0±5.0%), Vasc (76.9±7.1%), Microglia (78.6±7.1%), Olig (75.4±6.6%), and OPCs (77.4±8.3%). (FIG. 6E) t-SNE plot of 32,335 nuclei from V1 cortex of two injected animals. The gradient denotes number of unique viral transcripts per nucleus displayed as Log 10(Count+1). (FIG. 6F) Dot plot of the number of viral genomes in the AAV library and the number of infected cells recovered after snRNA-Seq. Each dot represents one barcode (n=861). Line of linear fit with 95% confidence intervals (shaded). Pearson correlation r=0.9, p<2.2×10⁻¹⁶.

FIG. 7 shows t-SNE plots of 32,335 nuclei from V1 cortex of two analyzed animals. The gradient denotes number of unique transcripts per nucleus of the indicated cellular marker gene.

FIG. 8 is a dot plot of pairwise comparison between SST fold-enrichment values across three sets of barcodes. The values on each axis represent the SST fold-enrichment calculated for each GRE based on one of the three barcodes paired with that GRE. The line indicates linear fit with 95% confidence intervals (shaded). Correlation and p-values are indicated for each plot. The gradient indicates the average enrichment between all three barcodes.

FIG. 9A-9B shows a series of plots. (FIG. 9A) t-SNE plot of 32,335 nuclei from V1 cortex of two analyzed animals showing the mean viral expression across all GREs. Plot is pseudocolored based on the mean expression in each cell type. (FIG. 9B) Volcano plots for identified SST-enriched GREs (Fold-enrichment>7 and FDR<0.01). The light grey dots represent the five SST-enriched GREs that were considered hits.

FIG. 10 shows fluorescent images from adult Sst-Cre; Ai14 mouse visual cortex twelve days following injection with rAAV-GRE-GFP as indicated.

FIG. 11 is a plot showing quantification of the number of GFP⁺ SST⁺ cells normalized for area of infection. Each dot represents one animal. Box plot represents mean±standard error of the mean (s.e.m). Values are 73.0±17.2, 146.9±19.7, 144.8±38.6 and 125.6±26.4 cells/mm² for AAV-[ΔGRE, GRE12, GRE22, GRE44]-GFP, respectively.

FIG. 12 shows fluorescent images from adult Vip-Cre; Ai14 mouse visual cortex immunostained for PVALB twelve days following injection with rAAV-GRE-GFP as indicated.

FIG. 13 is a line graph showing the number of new AAV clinical trials from approximately 1990 until 2018.

FIG. 14A-14B is a series of schematics explaining capsid engineering and expression engineering of viral vectors. FIG. 14A is a schematic explaining capsid engineering. The tissue and cell-type tropism of the virus is determined by the protein capsid. FIG. 14B is a schematic explaining expression engineering of viral vectors. After the cell is infected, the expression of the therapeutic payload is driven by the chosen regulatory element.

FIG. 15 is a schematic comparing capsid engineering and expression engineering of viral vectors. The nine viral vectors shown on the left represent capsid engineering as they all comprise the same genetic material but different capsids. The nine viral vectors shown on the right represent expression engineering as they all comprise the same capsid but genetic materials.

FIG. 16 is a schematic showing the expression engineering platform described herein, comprising the following steps: identify candidate regulatory elements; generate AAV library of barcoded regulatory element reporters; screen for enhancer expression across search space; and analyze and confirm tissue-type-specific AAVs or cell-type-specific AAVs.

FIG. 17 is a series of images showing the test of control unaltered AAV and altered AAV identified using the platform described herein.

FIG. 18 is a series of graphs showing a CNO-responsive payload.

FIG. 19A-19B is a series of schematics showing the experimental strategy and GRE selection. (FIG. 19A) Paralleled Enhancer Single Cell Assay (PESCA). Comparative ATAC-Seq is used to identify candidate GREs. A library of gene regulatory elements (GREs) is inserted upstream of a minimal promoter-driven GFP. The viral barcode sequence is inserted in the 3′UTR, and the vector packaged into rAAVs. Following en masse injection of the rAAV library, the specificity of the constituent GREs for various cell types in vivo is determined by single-nucleus RNA sequencing, measuring expression of the barcoded transcripts in tens of thousands of individual cells in the target tissue. Finally, bioinformatic analysis determines the most cell-type-specific barcode-associated rAAV-GRE-GFP constructs. pA=polyA tail. (FIG. 19B) Area-proportional Venn diagram of the number of putative GREs identified by ATAC-Seq of purified PV, SST, and VIP nuclei. Overlapping areas indicate shared putative GREs. Non-overlapping areas represent GREs that are unique to a single cell type.

FIG. 20 is a heatmap showing hierarchical clustering of the Mo et al. (2015) dataset and the ATAC-seq dataset described herein. Any ATAC-seq peak identified in any of the PV, SST, or VIP ATAC-seq datasets of this manuscript was given a score of 0 or one depending on whether any reads fell into that peak for a given sample. A binary score was used rather than normalized read counts to account for batch effects (due to differences in sample preparation, processing, and sequencing depth) between Mo et al.'s dataset and the dataset described herein. The pairwise correlation coefficient of these binary vectors was then calculated for each possible combination of samples shown, and hierarchically clustered using (R\hat{ }2) as the distance metric.

FIG. 21 is a dot plot with each dot representing one GRE (n=287). The values on each axis represent the Log 2 SST fold-enrichment calculated for each GRE based on two of the three barcodes paired with that GRE—barcode one on the x-axis, and barcode three on the y-axis. Blue line indicates linear fit with 95% confidence intervals (shaded) (r=0.55, p<2.2×10⁻¹⁶, Pearson's correlation). Gradient indicates the average enrichment between the two barcodes.

FIG. 22 is a plot showing the density distribution of number of UMIs and genes per nucleus.

FIG. 23 is a series of bar graphs showing mean expression of GRE12, GRE19, GRE22, GRE44, and GRE80 across cell types. Error bars, s.e.m.

FIG. 24 is a series of dot plots showing pairwise comparison between SST fold-enrichment values. Dot plot of pairwise comparison between SST fold-enrichment values across three pairs of barcodes associated with the same GRE (left) and across randomly shuffled barcodes (right). The values on each axis represent the Log 2 SST fold-enrichment calculated for each barcode. Line indicates linear fit with 95% confidence intervals (shaded). Correlation and p-values are indicated for each plot. Gradient indicates the average enrichment between the two barcodes.

FIG. 25 is a scatter plot of between Log 2 SST fold-enrichment values across two animals. Line indicates linear fit with 95% confidence intervals (shaded). Correlation and p-values are indicated for each plot. Gradient indicates the average enrichment between the two barcodes.

FIG. 26 is a cumulative bar plot of fold SST enrichment. Each bar represents three barcodes (shaded differently) associated with one GRE. GREs on the X-axis ranked by cumulative enrichment.

FIG. 27 is a scatter plot of GRE-driven transcripts plotted as Log 10 transcript count by fold SST-specificity. Dots represent all GREs that were considered statistically enriched in SST+ cells (FDR corrected q<0.05).

FIG. 28A-28D is a series of graphs showing analysis of computationally subsampled data. Data from each of the five most cell-type-specific GRE hits was computationally subsampled to decrease the number of viral transcripts by 2, 4, 8, or 16 fold (x-axis) (see e.g., Materials and methods). Each simulation was run ten times. The number of viral transcripts following subsampling (FIG. 28A), the fold specificity for SST cells (FIG. 28B), and the FDR-corrected q value of the enrichment in SST cells (FIG. 28C) is plotted on the y-axis for each GRE as a function of the subsampling factor. FIG. 28D shows the scatter plot of the statistical enrichment as a function of the number of viral transcripts across all of the subsampling simulation. Dashed line indicates q=0.05. Gray line indicates linear fit with 95% confidence intervals (shaded, Pearson correlation, r=0.70, p<2.2*10⁻¹⁶).

FIG. 29 is a series of graphs showing distribution of the location of GFP-expressing cells as function of distance from the pia. Far left graph represents SST+ cells (n=2648); remaining lines represent GFP+ SST+ cells (n=2066, 172, 1164, and 765, respectively, for AAV-[DGRE, GRE12, GRE22, GRE44]-GFP). Shading represents the 95% confidence interval.

FIG. 30A-30B is a series of images and graphs showing Analysis of mDlx5/6-GFP+ cells. (FIG. 30A) Fluorescent images from adult Sst-Cre; Ai14 mouse visual cortex immunostained for PVALB twelve days following injection with rAAV-mDlx5/6-GFP as indicated. Scale bar 100 mm. (FIG. 30B) Quantification of the fraction of GFP+ cells that are SST+ and PVALB+. Each dot represents one animal. Box plot represents mean±standard error of the mean (s.e.m). Values are 42.9±3.9%, and 46.7±5.6% for SST+ and PVALB+ respectively.

FIG. 31 is a series of plots showing qquantification of the fraction of GFP+ cells that are present it each cortical layer. Each dot represents one animal. Box plot represents mean±standard error of the mean (s.e.m). Gray represents all SST+ cells, colored plots represent GFP+SST+ cells respectively, for AAV-[GRE12, GRE22, GRE44]-GFP).

FIG. 32A-32D is a series of graphs showing the electrophysiology of neurons expressing an rAAV-GRE-driven reporter and modulation of neuronal activity with rAAV-GREs. (FIG. 32A) Representative current-clamp recordings from SST neurons in the visual cortex of Sst-Cre; Ai14 mice injected with rAAV-GRE44-GFP. Top: Representative traces from a cortical SST neuron with Cre-dependent expression of tdTomato, in response to 1000 ms depolarizing current injections as indicated in black (‘GRE44−”). Bottom: Traces from a tdTomato+ SST neuron with GRE44-driven expression of GFP (‘GRE44+”). GRE44− SST neurons were only recorded in the immediate vicinity of GRE44+ SST neurons. (FIG. 32B) Recordings from GRE44+ and GRE44− neurons in response to hyperpolarizing, 1000 ms currents. Asterisks indicate the sag likely due to the hyperpolarization-activated current Iₕ. Rebound action potentials following recovery from hyperpolarization, likely due to low-threshold calcium spikes mediated by T-type calcium channels, were also present in cells of both groups. Same scale as FIG. 32A. (FIG. 32C) Broader action potentials in GRE44+SST neurons (bottom) compared to GRE44− SST neurons (top). Same vertical scale as FIG. 32A-32B. (FIG. 32D) Electrophysiological properties that differ between GRE44+ (n=16 cells from five mice) and GRE44− (n=16 cells from four mice) SST neurons, including rheobase (minimal amount of current necessary to elicit a spike), maximal rate of rise during the depolarizing phase of the action potential, the initial and steady state firing frequencies (both measured at the maximal current step before spike inactivation), and spike width (measured as the width at half-maximal spike amplitude). *p<0.05; ***p<0.001, unpaired t-test, two-tailed.

FIG. 33A-33C is a series of graphs and images showing the electrophysiology of neurons expressing an rAAV-GRE-driven reporter and modulation of neuronal activity with rAAV-GREs. (FIG. 33A) Representative recordings from nearby uninfected pyramidal neurons in the visual cortex of mice that were injected with AAV-GRE-12-Gq-tdTomato+, before (top) and during CNO application (bottom). (FIG. 33B) Firing rates of pyramidal neurons during CNO application remain unchanged (three animals, 5 cells). ns, p>0.05, paired t-test, two-tailed. (FIG. 33C) Representative image of a nearby recorded uninfected pyramidal neuron that was filled with neurobiotin.

### DETAILED DESCRIPTION

Gene therapy approaches are limited by non-specificity across cell types and there is a great need in the art to target individual cell types. Towards this end, the Inventors developed a platform that allows us to rapidly generate cell-type-specific viruses, including for examples AAVs specific for the brain. Briefly, the process begins by generating thousands of AAV variants which vary in the DNA sequence that drives the payload expression. Then, one can test in a single experiment the specificity of all of the AAVs in the tissue of interest using a new single-cell sequencing platform that allows us to quantify the levels of each virus across 10,000s of individual cells in the tissue. Instead of testing one virus at a time using fluorescence microscopy, the Inventors replaced the microscope with a sequencing technology so one can evaluate 100s or 1000s of AAVs simultaneously, and develop target-specific viruses within only a few months. Importantly, this is the first platform of its kind and it can easily be applied to a variety of tissues. Initial studies showed that virus with <10% on-target expression and developed a variant with >90% specificity for a rare brain cells type. Such approaches can be widely extended to develop viruses to target other cells types in the brain as well as, the retina, and the inner ear.

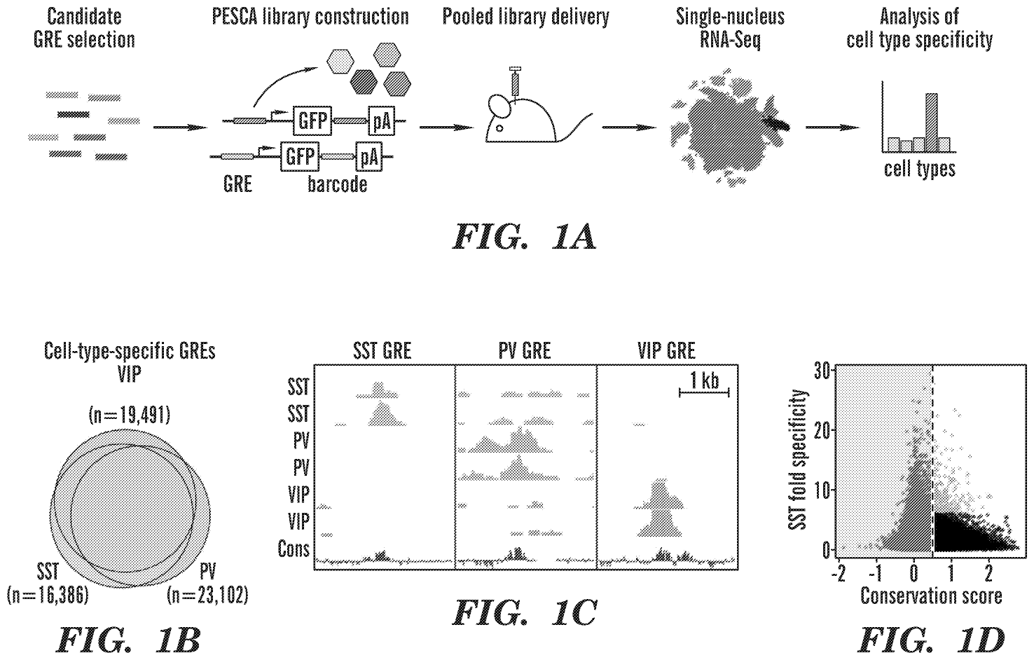

This platform, described herein as scalable Paralleled Enhancer Single Cell Assay (PESCA), assesses the specificity of viral vectors across the full complement of cell types present in the target tissue. More specifically, barcoded AAV vectors harboring putative cell-type-restricted enhancer elements are packaged for delivery. Following injection of the pooled AAV-packaged library, single-nucleus RNA sequencing (snRNA-seq) is used to evaluate the specificity of the constituent GREs for various cell types, measuring expression of the complement of GFP barcodes expressed in tens of thousands of individual cells in the target tissue while preserving the cell type identity of each cell through the use of an orthogonal cell-indexed system of transcript barcoding (see e.g., FIG. 1A).

Validation of this approach was achieved by applying the PESCA platform to address a central challenge in modern neuroscience: the limited ability to access functionally and molecularly distinct neuronal subtypes for targeted observation and functional perturbation. The Inventors generated and screened a library of 287 GREs in mice and identified among the top PESCA hits two enhancers capable of restricting AAV gene expression to a subset of somatostatin (SST)-expressing interneurons, thus highlighting the utility of PESCA as a platform to generate cell-type-specific AAVs that will be of broad interest to the scientific community. Given that previous viral drivers have been found to largely retain their specificity across several species, this strategy provides new tools for use in genetically inaccessible model organisms, with important implications for future therapeutic applications in human patients.

Described herein is a vector. In some embodiments of any of the aspects, the vector includes viral elements, such as viruses including adeno-associated virus (AAV) and lentivirus. In some embodiments of any of the aspects, the vector, includes at least one inverted terminal repeat, at least one gene regulatory element (GRE), an expression cassette, and a polyadenylation tail. In some embodiments of any of the aspects, the vector is an adeno-associated virus (AAV) vector, In some embodiments of any of the aspects, the at least one GRE exhibits cell-type specificity. In some embodiments of any of the aspects, the at least one GRE is primate, such as human. In some embodiments of any of the aspects, the at least one GRE is selected from the group consisting of: GRE12, GRE19, GRE22, GRE44, and GRE80. In some embodiments of any of the aspects, the AAV is selected from the group consisting of: bovine AAV (b-AAV), canine AAV (CAAV), mouse AAV1, caprine AAV, rat AAV, avian AAV (AAAV), AAV1, AAV2, AAV3b, AAV4, AAV5, AAV6, AAV7, AAV8, AAV9, AAV10, AAV11, AAV12, and AAV13. In some embodiments of any of the aspects, the AAV vector encodes an AAV capsid without a functional Rep protein. In some embodiments of any of the aspects, the AAV vector encodes an AAV capsid without one or more of VP1, VP2 and VP3. In some embodiments of any of the aspects, a host cell includes the aforementioned vector, including AAV vector.

Also described herein is a method of screening. In some embodiments of any of the aspects, the method of screening is for viral cell type specificity. In some embodiments of any of the aspects, the virus is adeno-associated virus (AAV), lentivirus, etc. In some embodiments of any of the aspects, the viral cell type specificity is adeno-associated virus (AAV) cell-type specific gene regulatory elements (GREs), including labeling a library of GREs with barcodes including a nucleic acid, wherein each of the barcodes is associated with a GRE structure, function, or both, in the library of GREs, packaging the library of labeled GREs into AAV to generate an AAV library, administering the AAV library to an organism, detecting the barcodes in one or more cell types in the organism, and identifying the GRE based on the cell type of interest and detected barcodes, thereby screening cell-type specific GREs. In some embodiments of any of the aspects, labeling the library of GREs includes amplifying GREs using polymerase chain reaction (PCR) with a primer including a vector cloning site, a barcode sequence. In some embodiments of any of the aspects, the barcode sequence is about 7-15 base pairs. In some embodiments of any of the aspects, the barcode is 10 base pairs. In some embodiments of any of the aspects, packaging the library of labeled GREs into the AAV library includes shuttling of the GRE PCR products into an AAV vector. In some embodiments of any of the aspects, detecting the barcodes in one or more cell types in the organism includes single cell RNA sequencing (sc-RNA seq) or single nucleus RNA sequencing (sn-RNA seq). In some embodiments of any of the aspects, detecting the barcodes in single cells in the organism includes single cell RNA sequencing (sc-RNA seq). In some embodiments of any of the aspects, each of the barcodes is unique to a GRE in the library of GREs. In some embodiments of any of the aspects, detecting the barcodes in one or more cell types in the organism includes enrichment of RNA transcripts. In some embodiments of any of the aspects, enrichment of RNA transcripts includes reverse transcribing RNA transcripts to generate complementary DNA (cDNA), amplifying the cDNA using second strand synthesis, and transcription of the cDNA to generate RNA intermediates. In some embodiments of any of the aspects, the RNA intermediates are amplified using PCR. In some embodiments of any of the aspects, detecting the barcodes in one or more cell types in the organism includes capturing nuclei of the one or more cell types in hydrogels including cell barcode single primers.

In some embodiments of any of the aspects, the method of screening is for capsid sequences. In some embodiments of any of the aspects, one or more, including a library, of capsid DNA is encoded in viral genome and its expression detected in scRNA-seq to ID the cell-type-specificity and magnitude of expression of each virus carrying a unique capsid. In some embodiments of any of the aspects, capsids are barcoded to generate a library of capsids detected as one or more, including a library of barcodes. In some embodiments of any of the aspects, capsids include a variable region modified to generate the library of capsids detected as one or more, including a library of barcodes. In some embodiments of any of the aspects, the one or more barcodes is associated with a capsid structure, function, or both.

Also described herein is a method of detecting expression level of viral related genetic elements. In some embodiments of any of the aspects, the virus is adeno-associated virus (AAV), lentivirus, etc. In some embodiments of any of the aspects, the viral related genetic elements include adeno-associated virus (AAV) gene regulatory elements (GREs), including labeling a library of GREs with barcodes including a nucleic acid, wherein each of the barcodes is associated with a GRE structure, function, or both, in the library of GREs, packaging the library of labeled GREs into AAV to generate an AAV library, administering the AAV library to an organism, detecting the barcodes in one or more cell types in the organism, and identifying the GRE based on detected barcodes, thereby detecting expression levels associated with the viral related genetic elements. In some embodiments of any of the aspects, labeling the library of GREs includes amplifying GREs using polymerase chain reaction (PCR) with a primer including a vector cloning site, a barcode sequence. In some embodiments of any of the aspects, the barcode sequence is about 7-15 base pairs. In some embodiments of any of the aspects, the barcode is 10 base pairs. In some embodiments of any of the aspects, packaging the library of labeled GREs into the AAV library includes shuttling of the GRE PCR products into an AAV vector. In some embodiments of any of the aspects, detecting the barcodes in one or more cell types in the organism includes single cell RNA sequencing (sc-RNA seq) or single nucleus RNA sequencing (sn-RNA seq). In some embodiments of any of the aspects, detecting the barcodes in single cells in the organism includes single cell RNA sequencing (sc-RNA seq). In some embodiments of any of the aspects, each of the barcodes is unique to a GRE in the library of GREs. In some embodiments of any of the aspects, detecting the barcodes in one or more cell types in the organism includes enrichment of RNA transcripts. In some embodiments of any of the aspects, enrichment of RNA transcripts includes reverse transcribing RNA transcripts to generate complementary DNA (cDNA), amplifying the cDNA using second strand synthesis, and transcription of the cDNA to generate RNA intermediates. In some embodiments of any of the aspects, the RNA intermediates are amplified using PCR. In some embodiments of any of the aspects, detecting the barcodes in one or more cell types in the organism includes capturing nuclei of the one or more cell types in hydrogels including cell barcode single primers.

Further described herein is a composition, including: a nucleic acid sequence at least 10%, 20%, 30%, 40%, 50%, 60%, 70%, 80%, 85%, 90%, 95%, 96%, 97%, 98%, 99% or 100% identical to part or whole of one of sequence GRE12, GRE19, GRE22, GRE44 or GRE80.

### Vectors

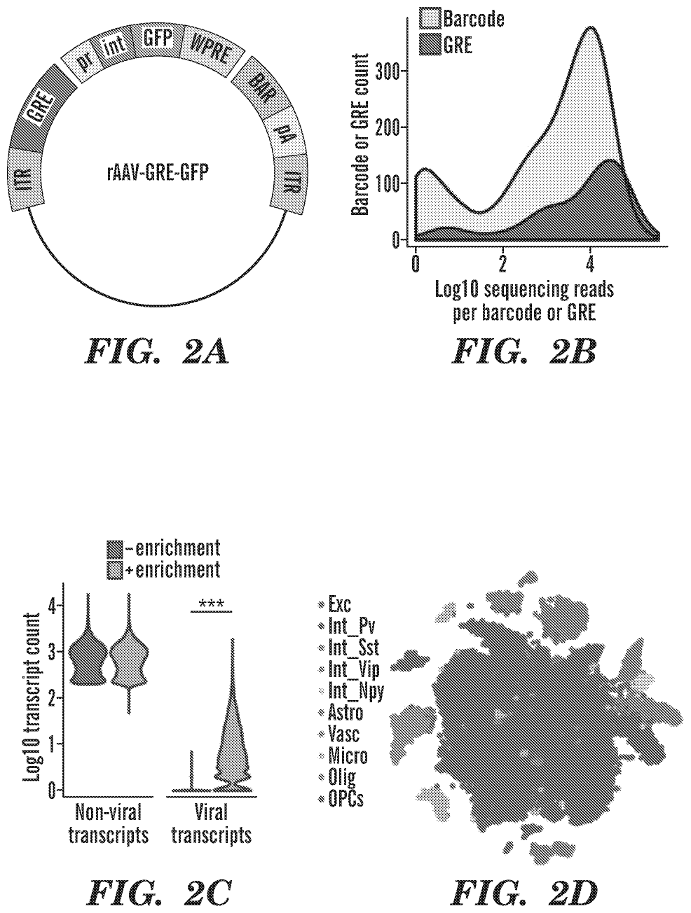

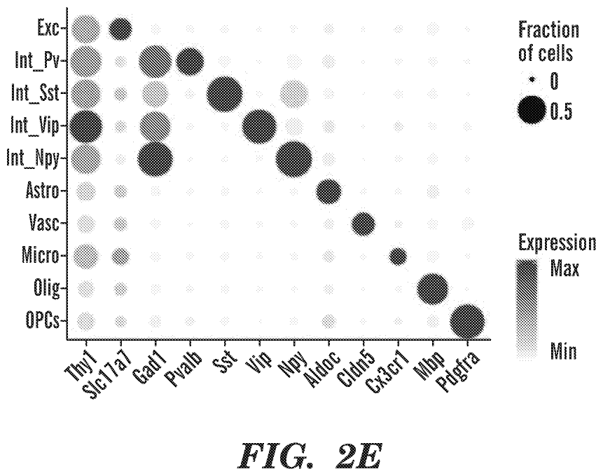

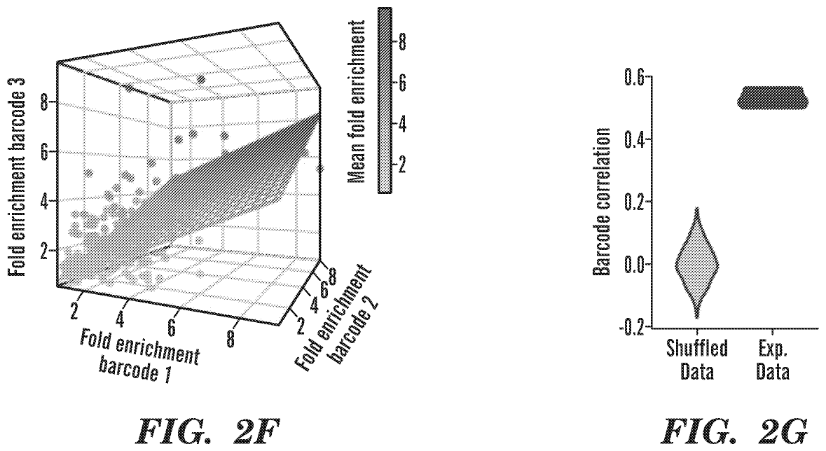

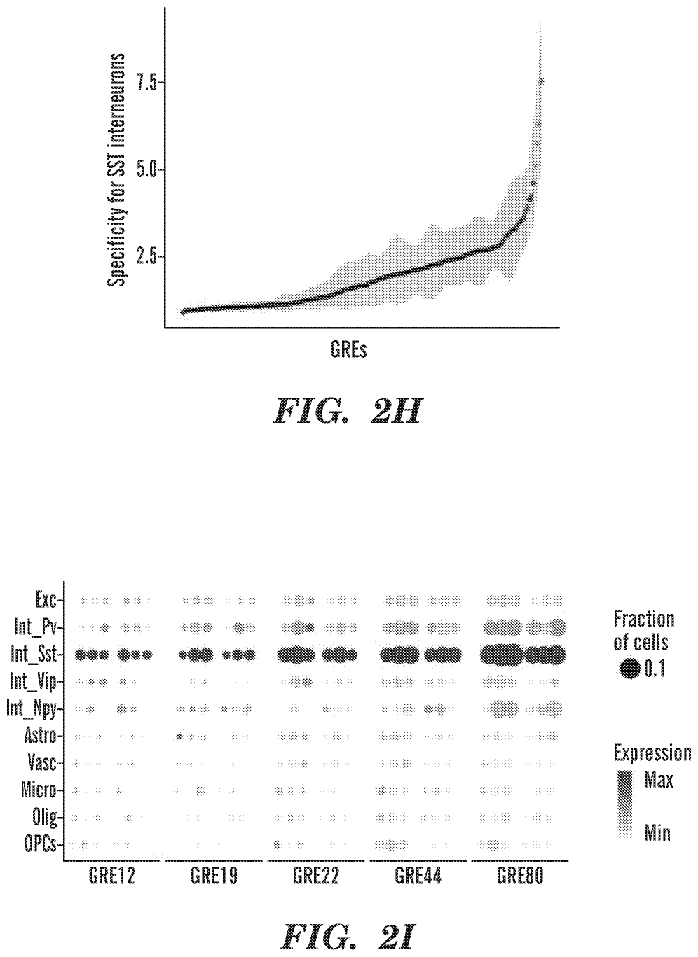

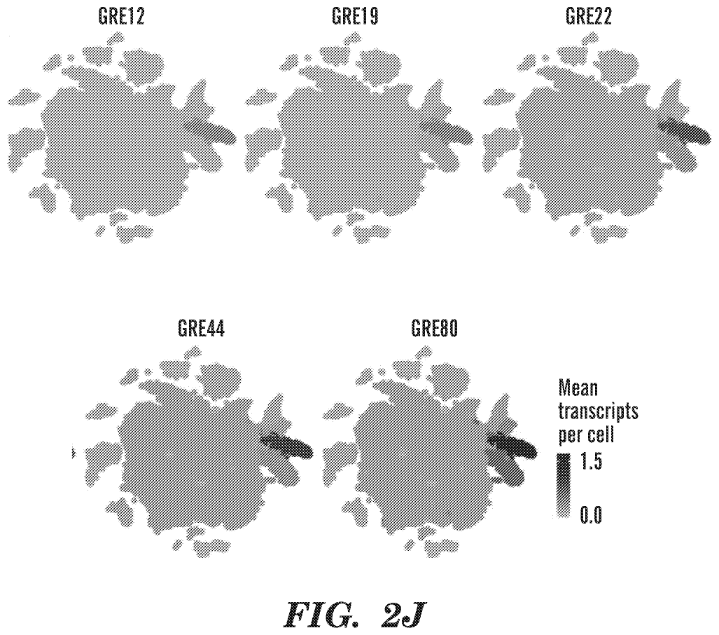

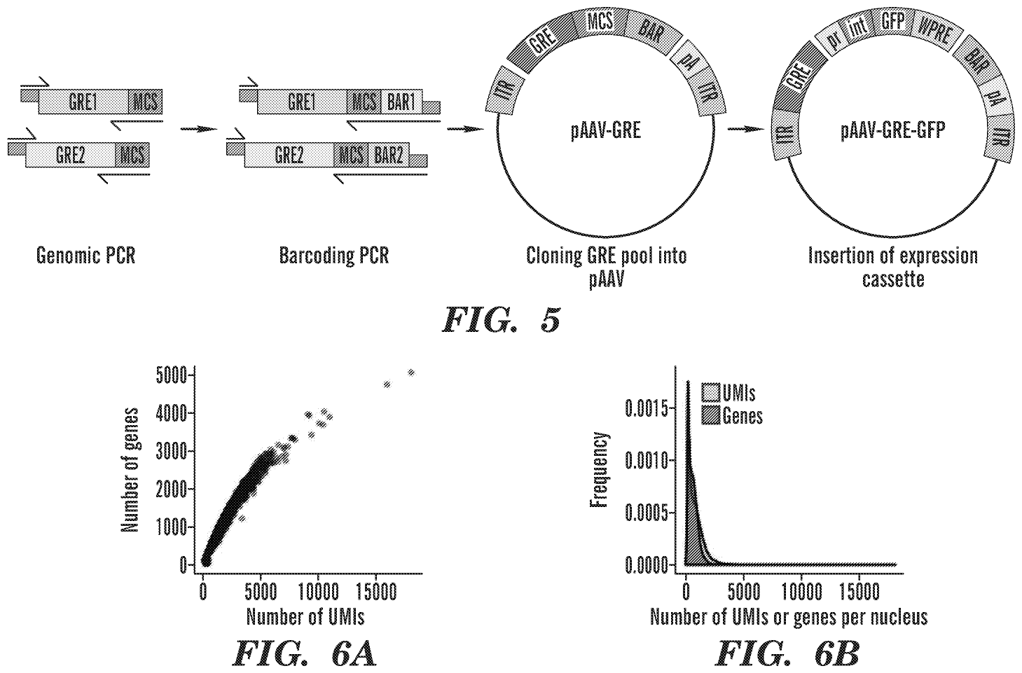

Described herein is a vector. In some embodiments of any of the aspects, the vector includes viral elements, such as viruses including adeno-associated virus (AAV) and lentivirus. In some embodiments of any of the aspects, the vector, includes at least one inverted terminal repeat (ITR), at least one gene regulatory element (GRE), an expression cassette, and a polyadenylation tail. In some embodiments of any of the aspects, the vector is an adeno-associated virus (AAV) vector. In some embodiments of any of the aspects, an exemplary vector is shown in FIG. 2A or FIG. 5.

In some embodiments of any of the aspects, the vector comprises at least one ITR. In some embodiments of any of the aspects, the vector comprises at least one ITR from bovine AAV (b-AAV), canine AAV (CAAV), mouse AAV1, caprine AAV, rat AAV, avian AAV (AAAV), AAV1, AAV2, AAV3b, AAV4, AAVS, AAV6, AAV7, AAV8, AAV9, AAV10, AAV11, AAV12, or AAV13. In some embodiments of any of the aspects, the ITR is approximately 145 bases long (e.g., approximately 140-150 bases, 130-160 bases, etc.). In some embodiments of any of the aspects, the ITR comprises symmetrical sequences, e.g., that allow for the formation of a hairpin. In some embodiments of any of the aspects, the ITR allows for at least the following functions: genome replication (e.g., self-priming that allows primase-independent synthesis of the second DNA strand), genome integration into the host cell genome, and/or efficient encapsidation of the AAV genome.

In some embodiments of any of the aspects, the vector comprises two ITRs. In some embodiments of any of the aspects, the vector comprises a 5′ ITR and a 3′ ITR. In some embodiments of any of the aspects, one ITR is 5′ to the GRE, expression cassette, and/or polyadenylation tail (or signal), and a second ITR is 3′ to the GRE, expression cassette, and/or polyadenylation tail (or signal). In some embodiments of any of the aspects, the vector comprises the italicized portion(s) of SEQ ID NO: 10, SEQ ID NO: 11, SEQ ID NO: 12, SEQ ID NO: 13, or a sequence that is at least 80% (e.g., at least 80%, at least 90%, at least 95%, at least 96%, at least 97%, at least 98%, or at least 99%) identical to the sequence of the italicized portion(s) of SEQ ID NOs: 10-13 that maintains the same functions as the italicized portion(s) of SEQ ID NOs: 10-13 (e.g., genome replication, genome integration, and/or encapsidation).

In some embodiments of any of the aspects, the vector comprises at least one GRE. As a non-limiting example, the vector comprises at least 1, at least 2, at least 3, at least 4, or at least 5 GREs. In some embodiments of any of the aspects, the at least one GRE is primate, such as human. In some embodiments of any of the aspects, the at least one GRE is murine, such as from Mus musculus. In some embodiments of any of the aspects, a GRE that is murine in origin also exhibits the same cell type specificity in another mammal (e.g., primate, human). In some embodiments of any of the aspects, the at least one GRE exhibits mammalian sequence conservation (e.g., in at least rodents and primates).

In some embodiments of any of the aspects, the at least one GRE exhibits cell-type specificity. In some embodiments of any of the aspects, the at least one GRE exhibits cell-type specificity for any cell type within an organism. In some embodiments of any of the aspects, the at least one GRE exhibits cell-type specificity for a cell from the nervous system, brain, cerebrum, cerebral hemispheres, diencephalon, the brainstem, midbrain, pons, medulla oblongata, cerebellum, the spinal cord, the ventricular system, choroid plexus, peripheral nervous system, see also: list of nerves of the human body, nerves, cranial nerves, spinal nerves, ganglia, enteric nervous system, sensory organs, sensory system, eye, cornea, iris, ciliary body, lens, retina, ear, outer ear, earlobe, eardrum, middle ear, ossicles, inner ear, cochlea, vestibule of the ear, semicircular canals, olfactory epithelium, tongue, taste buds, integumentary system, mammary glands, skin, subcutaneous tissue, immune system, muscular system, musculoskeletal system, bone, human skeleton, joints, ligaments, muscular system, tendons, digestive system, mouth, teeth, tongue, salivary glands, parotid glands, submandibular glands, sublingual glands, pharynx, esophagus, stomach, small intestine, duodenum, jejunum, ileum, large intestine, liver, gallbladder, mesentery, pancreas, anal canal and anus, blood cells, respiratory system, nasal cavity, pharynx, larynx, trachea, bronchi, lungs, diaphragm, urinary system, kidneys, ureter, bladder, urethra, reproductive organs, female reproductive system, internal reproductive organs, ovaries, fallopian tubes, uterus, vagina, external reproductive organs, vulva, clitoris, placenta, male reproductive system, internal reproductive organs, testes, epididymis, vas deferens, seminal vesicles, prostate, bulbourethral glands, external reproductive organs, penis, scrotum, endocrine system, pituitary gland, pineal gland, thyroid gland, parathyroid glands, adrenal glands, pancreas, circulatory system, heart, patent foramen ovale, arteries, veins, capillaries, lymphatic system, lymphatic vessel, lymph node, bone marrow, thymus, spleen, gut-associated lymphoid tissue, tonsils, or interstitium.

In some embodiments of any of the aspects, the at least one GRE exhibits cell-type specificity for a cell of the nervous system. In some embodiments of any of the aspects, the at least one GRE exhibits cell-type specificity for a glial cell of the nervous system (e.g., oligodendrocytes, astrocytes, ependymal cells, Schwann cells, microglia, or satellite cells). In some embodiments of any of the aspects, the at least one GRE exhibits cell-type specificity for a neuron. Neurons are polarized cells with defined regions consisting of the cell body, an axon, and dendrites, although some types of neurons lack axons or dendrites. Their purpose is to receive, conduct, and transmit impulses in the nervous system. Neurons can be classified a number of different ways: anatomical, physiological, and developmental. Anatomical classes are defined first by the location of the neuron in the nervous system. Neurons are further distinguished from each other by features which include dendritic and axon morphology. Anatomical features also include synaptic connectivity (e.g., inputs and outputs) and molecular phenotype (e.g., the particular neurotransmitters, receptors, and ion channels expressed by a neuron). Neurons can be classified by their physiological properties. This includes their general function (e.g., sensory, motor, interneuron). Functions can also include whether the neuron is a relay neuron or a local interneuron or whether it is involved in sensory processing or correction of motor responses. Physiological actions can also include the firing properties of the neuron (e.g., bursting, tonic, quiescent). Developmental classifications of neurons are based upon the lineage that the cell derives from. The number of neurons in a particular class can vary over orders of magnitude from individual neurons in some classes to millions of neurons in other classes.

In some embodiments of any of the aspects, the at least one GRE exhibits cell-type specificity for a specific type of neuron. In some embodiments of any of the aspects, the at least one GRE exhibits cell-type specificity for a unipolar neuron, a bipolar neuron, a multipolar neuron, or a pseudounipolar neuron. In some embodiments of any of the aspects, the at least one GRE exhibits cell-type specificity for an interneuron, a sensory neuron, a motor neuron.

In some embodiments of any of the aspects, the at least one GRE exhibits cell-type specificity for a specific type of interneuron. In some embodiments of any of the aspects, the at least one GRE exhibits cell-type specificity for a somatostatin-expressing cortical interneuron, a somatostatin-expressing interneuron, and/or a cortical interneuron. In some embodiments of any of the aspects, the at least one GRE exhibits cell-type specificity for SST (somatostatin-expressing) interneurons of the primary visual cortex. In some embodiments of any of the aspects, the at least one GRE exhibits cell-type specificity for a specific subset of somatostatin-expressing cortical interneurons. In some embodiments of any of the aspects, the at least one GRE exhibits cell-type specificity for a somatostatin (SST)-expressing interneurons, a vasoactive intestinal polypeptide (VIP)-expressing interneuron or a parvalbumin (PV)-expressing interneuron (e.g., in the cerebral cortex). In some embodiments of any of the aspects, the at least one GRE exhibits cell-type specificity for a cholecystokinin-expressing (CCK)-expressing interneuron.

In some embodiments of any of the aspects, the at least one GRE exhibits cell-type specificity for a cell of the cerebral cortex (e.g., the mammalian cerebral cortex). In some embodiments of any of the aspects, the at least one GRE exhibits cell-type specificity for a cell located in a specific layer or layers of the cerebral cortex, for example layer(s) I, II, III, IV, V, and/or VI. Layer I is the molecular layer, which contains very few neurons; layer II is the external granular layer; layer III is the external pyramidal layer; layer IV is the internal granular layer; layer V is the internal pyramidal layer; and layer VI is the multiform, or fusiform layer. In some embodiments of any of the aspects, the at least one GRE exhibits cell-type specificity for cells (e.g., SST interneurons) in layer IV and V of the cerebral cortex.

In some embodiments of any of the aspects, the at least one GRE exhibits cell-type specificity for a cell of the cerebral cortex, including but not limited to pyramidal neurons; glial cells; Cajal-Retzius cells; subpial granular layer cells; spiny stellate cells; small pyramidal neurons; stellate neurons; medium-size pyramidal neurons; non-pyramidal neurons (e.g., with vertically oriented intracortical axons); large pyramidal neurons; giant pyramidal cells (e.g., Betz cells); small spindle-like pyramidal neurons; multiform neurons; or GABAergic rosehip neurons.

In some embodiments of any of the aspects, the at least one GRE exhibits cell-type specificity for an excitatory neuron. In some embodiments of any of the aspects, the at least one GRE exhibits cell-type specificity for an inhibitory neuron. In some embodiments of any of the aspects, the at least one GRE exhibits cell-type specificity for a glutamatergic excitatory neuron cell type. In some embodiments of any of the aspects, the at least one GRE exhibits cell-type specificity for a GABAergic inhibitory interneuron cell type.

In some embodiments of any of the aspects, the at least one GRE exhibits cell-type specificity for neuron that produces a specific neurotransmitter, including but not limited to arginine, aspartate, glutamate, gamma-aminobutyric acid, glycine, D-serine, acetylcholine, dopamine, norepinephrine (noradrenaline), epinephrine (adrenaline), serotonin (5-hydroxytryptamine), histamine, phenethylamine, N-methylphenethylamine, tyramine, octopamine, synephrine, tryptamine, N-methyltryptamine, anandamide, 2-arachidonoylglycerol, 2-arachidonyl glyceryl ether, N-arachidonoyl dopamine, virodhamine, adenosine, adenosine triphosphate, or nicotinamide adenine dinucleotide.

In some embodiments of any of the aspects, the at least one GRE exhibits cell-type specificity for neuron that produces a specific neuropeptide, including but not limited to Bradykinin, Corticotropin-releasing hormone, Urocortin, Galanin, Galanin-like peptide, Gastrin, Cholecystokinin, Adrenocorticotropic hormone, Proopiomelanocortin, Melanocyte-stimulating hormones, Vasopressin, Oxytocin, Neurophysin I, Neurophysin II, Neuromedin U, Neuropeptide B, Neuropeptide S, Neuropeptide Y, Pancreatic polypeptide, Peptide YY, Enkephalin, Dynorphin, Endorphin, Endomorphin, Nociceptin/orphanin FQ, Orexin A, Orexin B, Kisspeptin, Neuropeptide FF, Prolactin-releasing peptide, Pyroglutamylated RFamide peptide, Secretin, Motilin, Glucagon, Glucagon-like peptide-1, Glucagon-like peptide-2, Vasoactive intestinal peptide, Growth hormone-releasing hormone, Pituitary adenylate cyclase-activating peptide, Somatostatin, Neurokinin A, Neurokinin B, Substance P, Neuropeptide K, Agouti-related peptide, N-Acetylaspartylglutamate, Cocaine- and amphetamine-regulated transcript, Bombesin, Gastrin releasing peptide, Gonadotropin-releasing hormone, or Melanin-concentrating hormone. In some embodiments of any of the aspects, the at least one GRE exhibits cell-type specificity for neuron that produces a specific gasotransmitter (i.e., a gaseous signaling molecule), including but not limited to Nitric oxide, Carbon monoxide, or Hydrogen sulfide

In some embodiments of any of the aspects, the at least one GRE is selected from the group consisting of: GRE12, GRE19, GRE22, GRE44, and GRE80. In some embodiments of any of the aspects, the GRE is at least 100 base pairs (bp) long. In some embodiments of any of the aspects, the GRE is at least 10 bp, at least 20 bp, at least 30 bp, at least 40 bp, at least 50 bp, at least 60 bp, at least 70 bp, at least 80 bp, at least 90 bp, at least 100 bp, least 110 bp, at least 120 bp, at least 130 bp, at least 140 bp, at least 150 bp, at least 160 bp, at least 170 bp, at least 180 bp, at least 190 bp, at least 200 bp, least 210 bp, at least 220 bp, at least 230 bp, at least 240 bp, at least 250 bp, at least 260 bp, at least 270 bp, at least 280 bp, at least 290 bp, at least 300 bp, at least 350 bp, at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1000 bp long.

In some embodiments of any of the aspects, the GRE is at most 500 base pairs (bp) long. In some embodiments of any of the aspects, the GRE is at most 10 bp, at most 20 bp, at most 30 bp, at most 40 bp, at most 50 bp, at most 60 bp, at most 70 bp, at most 80 bp, at most 90 bp, at most 100 bp, most 110 bp, at most 120 bp, at most 130 bp, at most 140 bp, at most 150 bp, at most 160 bp, at most 170 bp, at most 180 bp, at most 190 bp, at most 200 bp, most 210 bp, at most 220 bp, at most 230 bp, at most 240 bp, at most 250 bp, at most 260 bp, at most 270 bp, at most 280 bp, at most 290 bp, at most 300 bp, at most 350 bp, at most 400 bp, at most 450 bp, at most 500 bp, at most 550 bp, at most 600 bp, at most 650 bp, at most 700 bp, at most 750 bp, at most 800 bp, at most 850 bp, at most 900 bp, at most 950 bp, or at most 1000 bp long.

In some embodiments of any of the aspects, the GRE comprises SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or a sequence that is at least 80% (e.g., at least 80%, at least 90%, at least 95%, at least 96%, at least 97%, at least 98%, or at least 99%) identical to the sequence of SEQ ID NOs: 14-21 that maintains the same functions as SEQ ID NOs: 14-21 (e.g., cell-type specificity).

In some embodiments of any of the aspects, the vector comprises GRE12 (e.g., SEQ ID NO: 14, SEQ ID NO: 17), or a sequence that is at least 80% (e.g., at least 80%, at least 90%, at least 95%, at least 96%, at least 97%, at least 98%, or at least 99%) identical to the sequence of GRE12 (e.g., SEQ ID NO: 14, SEQ ID NO: 17) that maintains the same functions as GRE12 (e.g., SEQ ID NO: 14, SEQ ID NO: 17) (e.g., SST-interneuron specificity).

In some embodiments of any of the aspects, the vector comprises GRE22 (e.g., SEQ ID NO: 15, SEQ ID NO: 18), or a sequence that is at least 80% (e.g., at least 80%, at least 90%, at least 95%, at least 96%, at least 97%, at least 98%, or at least 99%) identical to the sequence of GRE22 (e.g., SEQ ID NO: 15, SEQ ID NO: 18) that maintains the same functions as GRE22 (e.g., SEQ ID NO: 15, SEQ ID NO: 18) (e.g., SST-interneuron specificity).

In some embodiments of any of the aspects, the vector comprises GRE44 (e.g., SEQ ID NO: 16, SEQ ID NO: 19), or a sequence that is at least 80% (e.g., at least 80%, at least 90%, at least 95%, at least 96%, at least 97%, at least 98%, or at least 99%) identical to the sequence of GRE44 (e.g., SEQ ID NO: 16, SEQ ID NO: 19) that maintains the same functions as GRE44 (e.g., SEQ ID NO: 16, SEQ ID NO: 19) (e.g., SST-interneuron specificity).

In some embodiments of any of the aspects, the vector comprises GRE19 (e.g., SEQ ID NO: 20), or a sequence that is at least 80% (e.g., at least 80%, at least 90%, at least 95%, at least 96%, at least 97%, at least 98%, or at least 99%) identical to the sequence of GRE19 (e.g., SEQ ID NO: 20) that maintains the same functions as GRE19 (e.g., SEQ ID NO: 20) (e.g., SST-interneuron specificity).

In some embodiments of any of the aspects, the vector comprises GRE80 (e.g., SEQ ID NO: 21), or a sequence that is at least 80% (e.g., at least 80%, at least 90%, at least 95%, at least 96%, at least 97%, at least 98%, or at least 99%) identical to the sequence of GRE80 (e.g., SEQ ID NO: 21) that maintains the same functions as GRE80 (e.g., SEQ ID NO: 21) (e.g., SST-interneuron specificity).

In some embodiments of any of the aspects, the vector comprises an expression cassette. In some embodiments of any of the aspects, the expression cassette comprises a promoter, a detectable label, and/or a therapeutic gene. In some embodiments of any of the aspects, the expression cassette comprises a promoter and a detectable label. In some embodiments of any of the aspects, the expression cassette comprises a promoter and a therapeutic gene. In some embodiments of any of the aspects, the expression cassette comprises a detectable label and a therapeutic gene. In some embodiments of any of the aspects, the expression cassette comprises a promoter, a detectable label, and a therapeutic gene.

In some embodiments of any of the aspects, the promoter is a constitutive promoter (i.e., essentially on at all times). In some embodiments of any of the aspects, the promoter is a regulated promoter, an inducible promoter, or a tissue-specific promoter. In some embodiments of any of the aspects, the promoter of the expression cassette is a mammalian promoter. In some embodiments of any of the aspects, the promoter is a promoter that functions in a mammal (e.g., rodent, primate). In some embodiments of any of the aspects, the promoter is selected from the list of known mammalian promoters in the Mammalian Promoter Database (MPromDb; available on the world wide web at bio.tools/mpromdb). In some embodiments of any of the aspects, the promoter is a human promoter. In some embodiments of any of the aspects, the promoter is a promoter that functions in a human. In some embodiments of any of the aspects, the promoter is human beta-globin promoter. In some embodiments of any of the aspects, the promoter drives expression in the specific cell type in which the at least GRE exhibits cell-type specificity. In some embodiments of any of the aspects, the promoter is selected from the group consisting of the CMV, EF1a, SV40, PGK1 (human or mouse), Ubc, human beta actin, CAG, TRE, UAS, Ac5, Polyhedrin, CaMKIIa, GAL1, TEF1, GDS, ADH1, CaMV35S, Ubi, H1, or U6 promoters.

In some embodiments of any of the aspects, the expression cassette of the vector comprises a detectable label. In some embodiments of any of the aspects, the expression cassette comprises a light-absorbing dye, a fluorescent dye, a radioactive label, or another detectable label as described further herein.

In some embodiments of any of the aspects, the expression cassette of the vector comprises at least one open reading frame. In some embodiments of any of the aspects, the expression cassette of the vector comprises at least one transgene (i.e., a gene which is artificially introduced into the vector). In some embodiments of any of the aspects, the expression cassette of the vector comprises at least one (e.g., at least 1, at least 2, at least 3) therapeutic gene(s). As used herein, the term “therapeutic gene” (also referred to herein as a therapeutic payload) refers to a gene that is capable of eliciting a therapeutic or preventative effect or encodes a protein that is capable of eliciting a therapeutic or preventative effect.

In some embodiments of any of the aspects, the therapeutic gene comprises a drug-inducible polypeptide. As a non-limiting example, the drug-inducible polypeptide comprises a designer receptor exclusively activated by designer drugs (DREADD), e.g., that is activated by a synthetic ligand, including but not limited to clozapine-N4-oxide (CNO) (see e.g., SEQ ID NO: 22). DREADDs are a viral payload that dynamically regulate neuronal activity in response to a synthetic ligand. See e.g., Zhu and Roth, Int J Neuropsychopharmacol. 2015 Jan., 18(1): pyu007; US20190083652A1; US20190083573A1; WO2017153995A1; WO2017132255A1; the contents of each of which are incorporated by reference herein in their entireties.

In some embodiments of any of the aspects, the therapeutic gene can be any suitable nucleotide sequence to produce a therapeutic effect, and need not necessarily comprise a complete naturally occurring DNA or RNA sequence. In some embodiments of any of the aspects, the therapeutic gene comprises a synthetic RNA/DNA sequence, a recombinant RNA/DNA sequence (i.e. prepared by use of recombinant DNA techniques), a cDNA sequence, or a partial genomic DNA sequence, including combinations thereof. In some embodiments of any of the aspects, the therapeutic gene comprises a coding region or portion thereof. In some embodiments of any of the aspects, the therapeutic gene comprises a non-coding region or portion thereof. In some embodiments of any of the aspects, the therapeutic gene can be in a sense orientation or in an anti-sense orientation; preferably, it is in a sense orientation.

In some embodiments of any of the aspects, the therapeutic gene can be capable of blocking or inhibiting the expression of a gene in the target cell. For example, the therapeutic gene can be an antisense sequence. The inhibition of gene expression using antisense technology is well known in the art. The therapeutic gene or a sequence derived therefrom may be capable of “knocking out” the expression of a particular gene in the target cell. There are several “knock out” strategies known in the art. Alternatively, the therapeutic gene can be capable of enhancing or inducing ectopic expression of a gene in the target cell. The therapeutic gene or a sequence derived therefrom may be capable of “knocking in” the expression of a particular gene. Non-limiting examples of suitable therapeutic genes include: sequences encoding cytokines, chemokines, hormones, antibodies, anti-oxidant molecules, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumor suppresser protein and growth factors, membrane proteins, vasoactive proteins and peptides, anti-viral proteins and ribozymes, and derivatives thereof (such as with an associated reporter group) and pro-drug activating enzymes.

In some embodiments of any of the aspects, the vector comprises a polyadenylation tail. Polyadenylation is the addition of a poly(A) tail to a messenger RNA. The poly(A) tail consists of multiple adenosine monophosphates; in other words, it is a stretch of RNA that has only adenine bases. The poly(A) tail is important for the nuclear export, translation, and stability of mRNA. In some embodiments of any of the aspects, the nucleic acid encoding the vector comprises a polyadenylation signal sequence (e.g., AAUAAA on the RNA).

In some embodiments of any of the aspects, the vector further comprises a barcode sequence, as described further herein.

In some embodiments of any of the aspects, the AAV is selected from the group consisting of: bovine AAV (b-AAV), canine AAV (CAAV), mouse AAV1, caprine AAV, rat AAV, avian AAV (AAAV), AAV1, AAV2, AAV3b, AAV4, AAV5, AAV6, AAV7, AAV8, AAV9, AAV10, AAV11, AAV12, and AAV13.

In some embodiments of any of the aspects, the AAV vector is at least 1,000 base pairs (bp) long. In some embodiments of any of the aspects, the AAV vector is at least 500 bp, at least 750 bp, at least 1000 bp long, at least 1500 bp, at least 2000 bp long, at least 2500 bp, at least 3000 bp long, at least 3500 bp, at least 4000 bp long, at least 4500 bp, at least 5000 bp, at least 5500 bp, or at least 6000 bp long. In some embodiments of any of the aspects, the AAV vector is at most 6,000 base pairs (bp) long. In some embodiments of any of the aspects, the AAV vector is at most 500 bp, at most 750 bp, at most 1000 bp long, at most 1500 bp, at most 2000 bp long, at most 2500 bp, at most 3000 bp long, at most 3500 bp, at most 4000 bp long, at most 4500 bp, at most 5000 bp long, at most 5500 bp, or most least 6000 bp long.

In some embodiments of any of the aspects, the vector comprises SEQ ID NO: 10, SEQ ID NO: 11, SEQ ID NO: 12, SEQ ID NO: 13, or a sequence that is at least 80% (e.g., at least 80%, at least 90%, at least 95%, at least 96%, at least 97%, at least 98%, or at least 99%) identical to the sequence of SEQ ID NOs: 10-13 that maintains the same infectivity (e.g., cell type-specific infectivity) as SEQ ID NOs: 10-13.

In some embodiments of any of the aspects, the AAV vector encodes an AAV capsid without a functional Rep protein. In some embodiments of any of the aspects, the AAV vector encodes an AAV capsid without one or more of VP1, VP2 and VP3. In some embodiments of any of the aspects, a host cell includes the aforementioned vector, including AAV vector. In some embodiments of any of the aspects, the vector comprises at least one ITR (i.e., in cis), and structural (cap) and packaging (rep) proteins are delivered in trans (e.g., by at least one additional vector).

In some embodiments of any of the aspects, the cap and/or rep proteins are from a parvovirus. In some embodiments of any of the aspects, the cap and/or rep proteins are from the same or different AAV as AAV vector described herein. In some embodiments of any of the aspects, the cap and/or rep proteins are from bovine AAV (b-AAV), canine AAV (CAAV), mouse AAV1, caprine AAV, rat AAV, avian AAV (AAAV), AAV1, AAV2, AAV3b, AAV4, AAV5, AAV6, AAV7, AAV8, AAV9, AAV10, AAV11, AAV12, and AAV13. In some embodiments of any of the aspects, the cap and/or rep proteins are chimeric proteins, i.e., comprising amino acid sequences from at least two or more parvoviruses.

In some embodiments, one or more of the genes (e.g., the expression cassette) described herein is expressed in a recombinant expression vector or plasmid. As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, the vector is pAAV. Without limitations, the genes or nucleic acids described herein can be included in one vector or separate vectors. For example, the GRE and/or the expression cassette can be included in the same vector.

In some embodiments, the GRE and/or the expression cassette gene can be included in a first vector, the capsid and/or rep genes can be included in at least one additional vector (e.g., a packaging plasmid). In some embodiments, one or more of the recombinantly expressed gene can be integrated into the genome of the cell.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments of any of the aspects, a viral vector as described herein is introduced into a cell through methods well known in the art (see e.g., Daya and Berns, Gene Therapy Using Adeno-Associated Virus Vectors, Clin Microbiol Rev. 2008 October; 21(4): 583-593). In some embodiments of any of the aspects, the invention includes packaging cells which may be cultured to produce packaged viral vectors of the invention. Methods related to AAVs and elements for manufacture of AAV vectors are known in the art; see e.g., U.S. Pat. Nos. 5,478,745; 5,622,856; 5,658,776; 5,872,005; 6,156,303; 6,440,742; 6,521,225; 6,660,514; 6,632,670; 6,943,019; 7,629,322; 8,007,780; 9,527,904; and U.S. Patent Application Numbers US 2005/0266567; US 2005/0287122; US 2013/0224836; US 2017/0130245; the contents of each of which are incorporated herein by reference in their entireties.

### Screening Methods

Also described herein is a method of screening. In some embodiments of any of the aspects, the method of screening is for viral cell type specificity. In some embodiments of any of the aspects, the virus is adeno-associated virus (AAV), lentivirus, etc.

Accordingly, in one aspect described herein is a method of screening for adeno-associated virus (AAV) cell-type specific gene regulatory elements (GREs), comprising: (a) labeling a library of GREs with barcodes comprising a nucleic acid, wherein each of the barcodes is associated with a GRE structure, function, or both, in the library of GREs; (b) packaging the library of labeled GREs into AAV to generate an AAV library; (c) administering the AAV library to an organism; (d) detecting the barcodes in one or more cell types in the organism; and (e) identifying the GRE based on the cell type of interest and detected barcodes, thereby screening cell-type specific GREs.

In some embodiments of any of the aspects, a method as described herein comprises labeling a library of GREs with barcodes comprising a nucleic acid. In some embodiments of any of the aspects, each barcode is associated with a GRE structure, a GRE function, or both a GRE structure and a GRE function, in the library of GREs. As used herein, the term “GRE structure” refers to a GRE with a specific structure, such as a specific sequence or a specific secondary structure. As used herein, the term “GRE function” refers to a GRE with a specific function, such a specific cell type specificity, as described further herein.

In some embodiments of any of the aspects, labeling the library of GREs includes amplifying GREs using polymerase chain reaction (PCR) with a primer including a vector cloning site, a barcode sequence. In some embodiments of any of the aspects, the barcode sequence is about 7-15 base pairs (e.g., about 7 bp, about 8 bp, about 9 bp, about 10 bp, about 11 bp, about 12 bp, about 13 bp, about 14 bp, or about 15 bp). In some embodiments of any of the aspects, the barcode is 10 base pairs long. In some embodiments of any of the aspects, the barcode sequences are at least three insertions, deletions, or substitutions apart from each other, e.g., to minimize the effects of sequencing errors on the correct identification of each barcode. In some embodiments of any of the aspects, the barcode is located 3′ of the GRE and expression cassette (see e.g., FIG. 2A, FIG. 5). In some embodiments of any of the aspects, each GRE is paired with at least 1 (e.g., at least 1, at least 2, at least 3, at least 4, or at least 5) unique barcode sequences. In other words, multiple vectors are constructed each comprising the same GRE and a different barcode.

In some embodiments of any of the aspects, a method as described herein comprises packaging the library of labeled GREs into AAV to generate an AAV library. In some embodiments of any of the aspects, packaging the library of labeled GREs into the AAV library includes shuttling of the GRE PCR products into an AAV vector. Methods of packaging an AAV library are well known in the art and described further herein.

In some embodiments of any of the aspects, a method as described herein comprises administering (e.g., an effective amount of) the AAV library to an organism. Non-limiting examples of organisms or subjects are described further herein, and can include but are not limited to a model organism such as a mouse or non-human primate, or alternatively a cell culture system such as a human, primate, or rodent cell culture system.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

In some embodiments of any of the aspects, at least 1×10¹¹ genome copies/mL of the AAV library is administered to an organism. In some embodiments of any of the aspects, at least 1×10¹ genome copies/mL, at least 1×10² genome copies/mL, at least 1×10³ genome copies/mL, at least 1×10⁴ genome copies/mL, at least 1×10⁵ genome copies/mL, at least 1×10⁶ genome copies/mL, at least 1×10⁷ genome copies/mL, at least 1×10⁸ genome copies/mL, at least 1×10⁹ genome copies/mL, at least 1×10¹⁰ genome copies/mL, at least 1×10¹¹ genome copies/mL, at least 1×10¹² genome copies/mL, at least 1×10¹³ genome copies/mL, at least 1×10¹⁴ genome copies/mL, or at least 1×10¹⁵ genome copies/mL of the AAV library is administered to an organism.

Methods of administering AAV to an organism are well known in the art and described further herein. Exemplary modes of administration include intravenous, subcutaneous, intradermal, intramuscular, and intraarticular administration, and the like, as well as direct tissue or organ injection, alternatively, intrathecal, direct intramuscular, intraventricular, intravenous, intraperitoneal, intranasal, or intraocular injections. In some embodiments of any of the aspects, the AAV is administered to the organism intracranially, for example into a specific brain region (e.g., cerebral cortex; V1 layer of the cerebral cortex). In some embodiments of any of the aspects, the AAV is administered stereotactically.

In some embodiments of any of the aspects, a method as described herein comprises detecting the barcodes in one or more cell types in the organism. In some embodiments of any of the aspects, detecting the barcodes in one or more cell types in the organism includes single cell RNA sequencing (sc-RNA seq) or single nucleus RNA sequencing (sn-RNA seq). In some embodiments of any of the aspects, detecting the barcodes in single cells in the organism includes single cell RNA sequencing (sc-RNA seq). In some embodiments of any of the aspects, each of the barcodes is unique to a GRE in the library of GREs. In some embodiments of any of the aspects, detecting the barcodes in one or more cell types in the organism includes enrichment of RNA transcripts. In some embodiments of any of the aspects, enrichment of RNA transcripts includes reverse transcribing RNA transcripts to generate complementary DNA (cDNA), amplifying the cDNA using second strand synthesis, and transcription of the cDNA to generate RNA intermediates. In some embodiments of any of the aspects, the RNA intermediates are amplified using PCR. In some embodiments of any of the aspects, detecting the barcodes in one or more cell types in the organism includes capturing nuclei of the one or more cell types in hydrogels including cell barcode single primers.

In some embodiments of any of the aspects, a method as described herein comprises identifying the GRE based on the cell type of interest and detected barcodes, thereby screening cell-type specific GREs. In some embodiments of any of the aspects, the cell type of interest is the specific cell type for which the GRE exhibits cell-type specificity.

In some embodiments of any of the aspects, the screening method comprises aspects of massively parallel reporter assays (MPRA) and aspects of single-cell RNA sequencing (scRNA-seq), e.g., in order to identify and functionally assess the specificity of hundreds of GREs across the full complement of cell types present in the brain. Methods of massively parallel reporter assays (MPRA) are well known in the art. See e.g., Hard et al., 2017, Nucleic Acids Research 45:11607-11621; Inoue et al., 2017, Genome Research 27:38-52; Meirtikov et al., 2012, Nature Biotechnology 30:271-277; Murtha et al., 2014, Nature Methods 11:559-565, Patwardhan et al., 2012 Nature Biotechnology 30:265-270; Shen et al., 2016, Genome Research 26:238-255; the contents of each of which are incorporated herein by reference in their entireties. Methods of single-cell RNA sequencing scRNA-seq) are well known in the art. See e.g., Cao et al., 2017, Science 357:661-667; Hrvatin et al., 2018, Nature Neuroscience 21:120-129, Klein et al., 2015, Cell 161:1187-1201; Macosko et al., 2015, Cell 161:1202-1214, Rosenberg et al., 2018, Science 360:176-182; Stroud et al., 2017, Cell 171:1151-1164; Tasic et al., 2018, Nature 563:72-78; Tasic et al., 2016, Nature Neuroscience 19:335-346; Zeisel et at, 2015, Science 347:1138-1142; the contents of each of which are incorporated herein by reference in their entireties.

In some embodiments of any of the aspects, the method of screening is for capsid sequences. In some embodiments of any of the aspects, one or more, including a library, of capsid DNA is encoded in viral genome and its expression detected in scRNA-seq to ID the cell-type-specificity and magnitude of expression of each virus carrying a unique capsid. In some embodiments of any of the aspects, capsids are barcoded to generate a library of capsids detected as one or more, including a library of barcodes. In some embodiments of any of the aspects, capsids include a variable region modified to generate the library of capsids detected as one or more, including a library of barcodes. In some embodiments of any of the aspects, the one or more barcodes is associated with a capsid structure, function, or both.

In some embodiments of any of the aspects, the method of screening for capsid sequences comprises substantially the same steps as screening for a cell-type specific GRE, comprising replacing the GRE sequence with a capsid sequence. In some embodiments of any of the aspects, the AAV vector comprises the capsid sequence. In some embodiments of any of the aspects, the AAV vector does not comprise the capsid sequence, and the capsid sequence is supplied by at least one additional vector or plasmid (e.g., a packaging plasmid). In some embodiments of any of the aspects, the capsid sequence comprises VP1, VP2 and VP3 and/or analogs thereof.

### Nucleic Acid Compositions

Further described herein is a composition, including: a nucleic acid sequence at least 10%, 20%, 30%, 40%, 50%, 60%, 70%, 80%, 85%, 90%, 95%, 96%, 97%, 98%, 99% or 100% identical to part or whole of one of sequence GRE12 (e.g., SEQ ID NO: 14, SEQ ID NO: 17), GRE19 (e.g., SEQ ID NO: 20), GRE22 (e.g., SEQ ID NO: 15, SEQ ID NO: 18), GRE44 (e.g., SEQ ID NO: 16, SEQ ID NO: 18), or GRE80 (e.g., SEQ ID NO: 21).

In some embodiments of any of the aspects, the nucleic acid sequence is at least 1,000 base pairs (bp) long. In some embodiments of any of the aspects, the nucleic acid sequence is at least 500 bp, at least 750 bp, at least 1000 bp long, at least 1500 bp, at least 2000 bp long, at least 2500 bp, at least 3000 bp long, at least 3500 bp, at least 4000 bp long, at least 4500 bp, at least 5000 bp, at least 5500 bp, or at least 6000 bp long. In some embodiments of any of the aspects, the nucleic acid sequence is at most 6,000 base pairs (bp) long. In some embodiments of any of the aspects, the nucleic acid sequence is at most 500 bp, at most 750 bp, at most 1000 bp long, at most 1500 bp, at most 2000 bp long, at most 2500 bp, at most 3000 bp long, at most 3500 bp, at most 4000 bp long, at most 4500 bp, at most 5000 bp long, at most 5500 bp, or most least 6000 bp long.

In some embodiments of any of the aspects, the GRE of the nucleic acid sequence comprises SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or a sequence that is at least 10%, 20%, 30%, 40%, 50%, 60%, 70%, 80%, 85%, 90%, 95%, 96%, 97%, 98%, 99% or 100% identical to the sequence of SEQ ID NOs: 14-21 that maintains the same functions as SEQ ID NOs: 14-21 (e.g., cell-type specificity).

In some embodiments of any of the aspects, the nucleic acid sequence comprises GRE12 (e.g., SEQ ID NO: 14, SEQ ID NO: 17), or a sequence that is at least 10%, 20%, 30%, 40%, 50%, 60%, 70%, 80%, 85%, 90%, 95%, 96%, 97%, 98%, 99% or 100% identical to the sequence of GRE12 (e.g., SEQ ID NO: 14, SEQ ID NO: 17) that maintains the same functions as GRE12 (e.g., SEQ ID NO: 14, SEQ ID NO: 17) (e.g., SST-interneuron specificity).

In some embodiments of any of the aspects, the nucleic acid sequence comprises GRE22 (e.g., SEQ ID NO: 15, SEQ ID NO: 18), or a sequence that is at least 10%, 20%, 30%, 40%, 50%, 60%, 70%, 80%, 85%, 90%, 95%, 96%, 97%, 98%, 99% or 100% identical to the sequence of GRE22 (e.g., SEQ ID NO: 15, SEQ ID NO: 18) that maintains the same functions as GRE22 (e.g., SEQ ID NO: 15, SEQ ID NO: 18) (e.g., SST-interneuron specificity).

In some embodiments of any of the aspects, the nucleic acid sequence comprises GRE44 (e.g., SEQ ID NO: 16, SEQ ID NO: 19), or a sequence that is at least 10%, 20%, 30%, 40%, 50%, 60%, 70%, 80%, 85%, 90%, 95%, 96%, 97%, 98%, 99% or 100% to the sequence of GRE44 (e.g., SEQ ID NO: 16, SEQ ID NO: 19) that maintains the same functions as GRE44 (e.g., SEQ ID NO: 16, SEQ ID NO: 19) (e.g., SST-interneuron specificity).

In some embodiments of any of the aspects, the nucleic acid sequence comprises GRE19 (e.g., SEQ ID NO: 20), or a sequence that is at least 10%, 20%, 30%, 40%, 50%, 60%, 70%, 80%, 85%, 90%, 95%, 96%, 97%, 98%, 99% or 100% identical to the sequence of GRE19 (e.g., SEQ ID NO: 20) that maintains the same functions as GRE19 (e.g., SEQ ID NO: 20) (e.g., SST-interneuron specificity).

In some embodiments of any of the aspects, the nucleic acid sequence comprises GRE80 (e.g., SEQ ID NO: 21), or a sequence that is at least 10%, 20%, 30%, 40%, 50%, 60%, 70%, 80%, 85%, 90%, 95%, 96%, 97%, 98%, 99% or 100% identical to the sequence of GRE80 (e.g., SEQ ID NO: 21) that maintains the same functions as GRE80 (e.g., SEQ ID NO: 21) (e.g., SST-interneuron specificity).

In some embodiments of any of the aspects, the nucleic acid sequence comprises a portion of GRE12 (e.g., SEQ ID NO: 14, SEQ ID NO: 17), GRE19 (e.g., SEQ ID NO: 20), GRE22 (e.g., SEQ ID NO: 15, SEQ ID NO: 18), GRE44 (e.g., SEQ ID NO: 16, SEQ ID NO: 18), or GRE80 (e.g., SEQ ID NO: 21). In some embodiments of any of the aspects, the nucleic acid sequence comprises a sequence that is at least 80% (e.g., at least 80%, at least 90%, at least 95%, at least 96%, at least 97%, at least 98%, or at least 99%) identical to a portion of GRE12 (e.g., SEQ ID NO: 14, SEQ ID NO: 17), GRE19 (e.g., SEQ ID NO: 20), GRE22 (e.g., SEQ ID NO: 15, SEQ ID NO: 18), GRE44 (e.g., SEQ ID NO: 16, SEQ ID NO: 18), or GRE80 (e.g., SEQ ID NO: 21). In some embodiments of any of the aspects, the portion of a GRE as described herein can comprise the middle 25% of the GRE sequence (i.e., a sequence comprising the midpoint of the sequence, sequence comprising 12.5% of the length of the sequence before the midpoint, and sequence comprising 12.5% of the length of the sequence after the midpoint). In some embodiments of any of the aspects, the nucleic acid sequence comprises positions 96-160 of SEQ ID NO: 14, positions 96-160 of SEQ ID NO: 15, positions 96-160 of SEQ ID NO: 16. In some embodiments of any of the aspects, the nucleic acid sequence comprises positions 280-466 of SEQ ID NO: 17, positions 270-450 of SEQ ID NO: 18, positions 270-450 of SEQ ID NO: 19, positions 264-440 of SEQ ID NO: 20, or positions 279-463 of SEQ ID NO: 21. In some embodiments of any of the aspects, the portion of a GRE as described herein can comprise at least the middle 25%, 30%, 35%, 40%, 45%, 50%, 55%, 60%, 65%, 70%, 75%, 80%, 85%, 90%, or 95% of the GRE sequence.

In some embodiments of any of the aspects, a composition as described herein further comprises a pharmaceutically acceptable carrier. In some embodiments, the technology described herein relates to a pharmaceutical composition comprising an AAV vector or nucleic acid comprising at least one GRE as described herein, and optionally a pharmaceutically acceptable carrier. In some embodiments, the active ingredients of the pharmaceutical composition comprise an AAV vector or nucleic acid comprising at least one GRE as described herein. In some embodiments, the active ingredients of the pharmaceutical composition consist essentially of an AAV vector or nucleic acid comprising at least one GRE as described herein. In some embodiments, the active ingredients of the pharmaceutical composition consist of an AAV vector or nucleic acid comprising at least one GRE as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (23) C₂-C₁₂ alcohols, such as ethanol; and (24) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. an AAV vector or nucleic acid comprising at least one GRE as described herein.

In some embodiments of any of the aspects, a nucleic acid sequence as described herein is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2- [wherein the native phosphodiester backbone is represented as —O—P—O—CH2-].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol. Canc. Ther. 6(3):833-843; Grunweller, A. et al., (2003) Nucleic Acids Research 31(12):3185-3193).

Modified nucleic acids can also contain one or more substituted sugar moieties. The nucleic acids described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO]mCH3, O(CH2)nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, nucleic acids include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments of any of the aspects, the modification includes a 2′ methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases can include other synthetic and natural nucleobases including but not limited to as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments of any of the aspects, oligonucleotide tags (e.g., Oligopaint) comprise any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

Non-limiting examples of genetic, tissue, or cell-specific disorders that can be treated using an AAV vector or nucleic acid as described herein include but are not limited to congenital deafness, ALS (Lou Gehrig's disease), cystic fibrosis, congenital bleeding disorders, congenital blindness, other forms of blindness, muscular dystrophies, alpha-1 antitrypsin deficiency, lysosomal storage disorders, Huntington disease, Rett syndrome, cardiovascular disease, osteoarthritis, macular degeneration, Alzheimer's disease, cancer, Parkinson's disease, and chronic pain (see e.g., Table 1).

### Detection Methods and Assays

Also described herein is a method of detecting expression level of viral related genetic elements. In some embodiments of any of the aspects, the virus is adeno-associated virus (AAV), lentivirus, etc. In some embodiments of any of the aspects, the viral related genetic elements include adeno-associated virus (AAV) gene regulatory elements (GREs), including labeling a library of GREs with barcodes including a nucleic acid, wherein each of the barcodes is associated with a GRE structure, function, or both, in the library of GREs, packaging the library of labeled GREs into AAV to generate an AAV library, administering the AAV library to an organism, detecting the barcodes in one or more cell types in the organism, and identifying the GRE based on detected barcodes, thereby detecting expression levels associated with the viral related genetic elements.

In some embodiments of any of the aspects, labeling the library of GREs includes amplifying GREs using polymerase chain reaction (PCR) with a primer including a vector cloning site, a barcode sequence (e.g., as described further herein). In some embodiments of any of the aspects, the barcode sequence is about 7-15 base pairs. In some embodiments of any of the aspects, the barcode is 10 base pairs. In some embodiments of any of the aspects, packaging the library of labeled GREs into the AAV library includes shuttling of the GRE PCR products into an AAV vector, as described further herein.

In some embodiments of any of the aspects, detecting the barcodes in one or more cell types in the organism includes single cell RNA sequencing (sc-RNA seq) or single nucleus RNA sequencing (sn-RNA seq). In some embodiments of any of the aspects, detecting the barcodes in single cells in the organism includes single cell RNA sequencing (sc-RNA seq). In some embodiments of any of the aspects, each of the barcodes is unique to a GRE in the library of GREs. In some embodiments of any of the aspects, detecting the barcodes in one or more cell types in the organism includes enrichment of RNA transcripts. In some embodiments of any of the aspects, enrichment of RNA transcripts includes reverse transcribing RNA transcripts to generate complementary DNA (cDNA), amplifying the cDNA using second strand synthesis, and transcription of the cDNA to generate RNA intermediates. In some embodiments of any of the aspects, the RNA intermediates are amplified using PCR. In some embodiments of any of the aspects, detecting the barcodes in one or more cell types in the organism includes capturing nuclei of the one or more cell types in hydrogels including cell barcode single primers.

In some embodiments of any of the aspects, measurement of the level of a target and/or detection of the level or presence of a target, e.g. of an expression product (e.g., expression level of viral related genetic elements) can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, e.g. an mRNA or polypeptide can comprise contacting a sample obtained from a subject with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

In certain embodiments, the nucleic acid can be detected by determining the level of nucleic acid in a sample. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNase protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, the level of a nucleic acid can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments of any of the aspects, one or more of the compositions described herein (e.g., an AAV vector, a nucleic acid sequence) can comprise a detectable label, can encode a detectable label, and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In some embodiments of any of the aspects, one or more of the compositions described herein (e.g., an AAV vector, a nucleic acid sequence) is labeled with or comprises a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthalaldehyde, fluorescamine, Cy3™, Cy5™, allophycocyanin, Texas Red, peridinin chlorophyll, cyanine, tandem conjugates such as phycoerythrin-Cy5™, green fluorescent protein (GFP), rhodamine, fluorescein isothiocyanate (FITC) and Oregon Green™, rhodamine and derivatives (e.g., Texas red and tetramethylrhodamine isothiocyanate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g., cyanine dyes such as Cy3, Cy5, etc.; BODIPY dyes and quinoline dyes. In some embodiments of any of the aspects, a detectable label can be a radiolabel including, but not limited to ³H, ¹²⁵I, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, one or more of the compositions described herein (e.g., an AAV vector, a nucleic acid sequence) can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromogenic substrate. Such streptavidin peroxidase detection kits are commercially available, e.g., from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylene diaminetetraacetic acid (EDTA).

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of expression of the target molecule in a control sample, a pooled sample of control individuals or a numeric value or range of values based on the same. In some embodiments of any of the aspects, the reference can be a level of expression of a AAV vector or a nucleic acid sequence not comprising a GRE as described herein (e.g., SEQ ID NO: 10). In some embodiments of any of the aspects, the reference can be the level of a target molecule in a sample obtained from the same subject at an earlier point in time.

In some embodiments of any of the aspects, the methods described herein comprises screening and/or detecting at least 2 different AAV vectors or nucleic acid sequences. In some embodiments of any of the aspects, the methods described herein comprises screening and/or detecting at least 2, at least 3, at least 4, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500 different AAV vectors or nucleic acid sequences comprising at least one GRE as described herein.

In some embodiments, the reference level can be the level in a sample of similar cell type, sample type, sample processing, and/or obtained from a subject of similar age, sex and other demographic parameters as the sample/subject for which the level of the AAV vector or nucleic acid sequence is to be determined. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

The test sample can be obtained by removing a sample from a subject, but can also be accomplished by using a previously isolated sample (e.g. isolated at a prior time point and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject or from an animal model as described herein.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present technology as disclosed herein, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data.

Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease selected for gene therapy. A subject can be male or female.

As used herein, the term “open reading frame” (ORF) refers to a sequence of nucleotides that, when read in a particular frame, do not contain any stop codons over the stretch of the open reading frame.

A “subject in need” of treatment for a particular condition can be a subject having that condition, diagnosed as having that condition, or at risk of developing that condition.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., viral DNA, genomic DNA, or cDNA. Suitable RNA can include, e.g., mRNA or viral RNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments of any of the aspects, the AAV vector or nucleic acid (e.g., comprising a GRE) described herein is exogenous. In some embodiments of any of the aspects, the AAV vector or nucleic acid (e.g., comprising a GRE) described herein is ectopic. In some embodiments of any of the aspects, the AAV vector or nucleic acid (e.g., comprising a GRE) described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments, a nucleic acid comprising a GRE as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul., 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in nucleic acid or protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements. In some embodiments of any of the aspects. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. An adeno-associated virus (AAV) vector, comprising:
   a. at least one inverted terminal repeat;
   b. at least one gene regulatory element (GRE);
   c. an expression cassette; and
   d. a polyadenylation tail.
2. The AAV vector of any one of the preceding paragraphs, wherein the at least one GRE exhibits cell-type specificity.
3. The AAV vector of any one of the preceding paragraphs, wherein the at least one GRE is selected from the group consisting of: GRE12, GRE19, GRE22, GRE44, and GRE80.
4. The AAV vector of any one of the preceding paragraphs, wherein the AAV is selected from the group consisting of: bovine AAV (b-AAV); canine AAV (CAAV); mouse AAV1; caprine AAV; rat AAV; avian AAV (AAAV); AAV1; AAV2; AAV3b; AAV4; AAV5; AAV6; AAV7; AAV8; AAV9; AAV10; AAV11; AAV12; and AAV13.
5. The AAV vector of any one of the preceding paragraphs, wherein the AAV vector encodes an AAV capsid without a functional Rep protein.
6. The AAV vector of any one of the preceding paragraphs, wherein the AAV vector encodes an AAV capsid without one or more of VP1, VP2 and VP3.
7. A host cell comprising the AAV vector of any one of the preceding paragraphs.
8. A method of screening for adeno-associated virus (AAV) cell-type specific gene regulatory elements (GREs), comprising:
   a. labeling a library of GREs with barcodes comprising a nucleic acid, wherein each of the barcodes is associated with a GRE structure, function, or both, in the library of GREs;
   b. packaging the library of labeled GREs into AAV to generate an AAV library;
   c. administering the AAV library to an organism;
   d. detecting the barcodes in one or more cell types in the organism; and
   e. identifying the GRE based on the cell type of interest and detected barcodes, thereby screening cell-type specific GREs.
9. The method of any one of the preceding paragraphs, wherein labeling the library of GREs comprises amplifying GREs using polymerase chain reaction (PCR) with a primer comprising a vector cloning site, a barcode sequence.
10. The method of any one of the preceding paragraphs, wherein the barcode sequence is about 7-15 base pairs.
11. The method of any one of the preceding paragraphs, wherein the barcode is 10 base pairs.
12. The method of any one of the preceding paragraphs, wherein packaging the library of labeled GREs into the AAV library comprises shuttling of the GRE PCR products into an AAV vector.
13. The method of any one of the preceding paragraphs, wherein detecting the barcodes in one or more cell types in the organism comprises single cell RNA sequencing (sc-RNA seq) or single nucleus RNA sequencing (sn-RNA seq).
14. The method of any one of the preceding paragraphs, wherein detecting the barcodes in single cells in the organism comprises single cell RNA sequencing (sc-RNA seq).
15. The method of any one of the preceding paragraphs, wherein each of the barcodes is unique to a GRE in the library of GREs.
16. The method of any one of the preceding paragraphs, wherein detecting the barcodes in one or more cell types in the organism comprises enrichment of RNA transcripts.
17. The method of any one of the preceding paragraphs, wherein enrichment of RNA transcripts comprises reverse transcribing RNA transcripts to generate complementary DNA (cDNA), amplifying the cDNA using second strand synthesis, and transcription of the cDNA to generate RNA intermediates.
18. The method of any one of the preceding paragraphs, wherein the RNA intermediates are amplified using PCR.
19. The method of any one of the preceding paragraphs, wherein detecting the barcodes in one or more cell types in the organism comprises capturing nuclei of the one or more cell types in hydrogels comprising cell barcode single primers.
20. A composition, comprising a nucleic acid sequence at least 70%, 80%, 85%, 90%, 95%, 96%, 97%, 98%, 99% or 100% identical to one of sequence GRE12, GRE19, GRE22, GRE44 or GRE80.

### EXAMPLES

### Example 1

A Scalable Platform for the Development of Cell-Type-Specific Viral Drivers

Experimental Methods

Mice: Animal experiments were approved and followed ethical guidelines. For INTACT the Inventors crossed Sst-IRES-Cre (The Jackson Laboratory™ Stock #013044), Vip-IRES-Cre (The Jackson Laboratory™ Stock #010908) and Pv-Cre (The Jackson Laboratory Stock #017320) with SUN1-2xsfGFP-6xMYC (The Jackson Laboratory™ Stock #021039) and used adult (6-12 wk old) male and female F1 progeny. For PESCA screening the Inventors used adult (6-10 wk) C57BL/6J (The Jackson Laboratory™, Stock #000664) mice. For confirmation of hits the Inventors crossed Sst-IRES-Cre (The Jackson Laboratory™ Stock #013044), Vip-IRES-Cre (The Jackson Laboratory™ Stock #031628) and Gad2-IRES-Cre (The Jackson Laboratory™ Stock #028867) mice with Ai14 mice (The Jackson Laboratory™ Stock #007914) and used adult (6-12 wk old) male and female F1 progeny. All mice were housed under a standard 12 hr light/dark cycle.

INTACT purification and in vitro transposition: INTACT employs a transgenic mouse that expresses a cell-type-specific Cre and a Cre-dependent SUN1-2xsfGFP-6xMYC (SUN1-GFP) fusion protein. Nuclear purifications were performed from whole cortex of adult mice as previously described using anti-GFP antibodies (Fisher G10362; see e.g., Mo et al., 2015, Neuron 86:1369-1384; Stroud et al., 2017, Cell 171:1151-1164). Isolated nuclei were gently resuspended in cold L1 buffer (50 mM Hepes pH 7.5, 140 mM NaCl, 1 mM EDTA, 1 mM EGTA, 0.25% Triton™ X-100, 0.5% NP40, 10% Glycerol, protease inhibitors), and pelleted at 800 g for 5 minutes at 4° C. DNA libraries were prepared from the nuclei using the Nextera™ DNA Library Prep Kit (Illumina™) according to manufacturer's protocols. The final libraries were purified using the Qiagen™ MinElute™ kit (Cat #28004) and sequenced on a Nextseg™ 500 benchtop DNA sequencer (Illumina™).

For each of the three inhibitory subtypes examined, two independent ATAC-seq experiments were performed, each on Sun1-positive nuclei isolated from a single animal. The nuclei were not counted prior to performing ATAC-seq, as yields were low enough that the process of counting would remove a large fraction of isolated nuclei and negatively impact the quality of the ATAC-seq experiment. However, during the process of establishing the Sun1 IP protocol, 20-30 k nuclei were consistently counted per animal

ATAC-seq mapping: All ATAC-seq libraries were sequenced on the Nextseg™ 500 benchtop DNA sequencer (Illumina™). Seventy-five base pair (bp) single-end reads were obtained for all datasets. ATAC-seq experiments were sequenced to a minimum depth of 20 million (M) reads. Reads for all samples were aligned to the mouse genome (GRCm38/mm10, December 2011) using default parameters for the Subread (subread-1.4.6-p3, (see e.g., Liao et al., 2013, Nucleic Acids Research 41:e108)) alignment tool after quality trimming with Trimmomatic™ v0.33 (see e.g., Bolger et al., 2014, Bioinformatics 30:2114-2120) with the following command: java -jar trimmomatic-0.33.jar SE -threads 1-phred33 [FASTQ_FILE] ILLUMINACLIP:[ADAPTER_FILE]:2:30:10 LEADING:5 TRAILING:5 SLIDINGWINDOW:4:20 MINLEN:45. Nextera adapters were trimmed out for ATAC-seq data. Duplicates were removed with samtools rmdup. To generate UCSC genome browser tracks for ATAC-seq visualization, BEDtools was used to convert output bam files to BED format with the bedtools bamtobed command. Published mm10 blacklisted regions (see e.g., Consortium, 2012; Schneider et al., 2017, Genome Research 27:849-864) were filtered out using the following command: bedops-not-element-of 1 [BLACKLIST_BED]. Filtered BED files were scaled to 20 M reads and converted to coverageBED format using the BEDtools genomecov command. bedGraphToBigWig (UCSC-tools) was used to generate bigWIG files for the UCSC genome browser.

ATAC-seq peak calling and quantification: Two independent peak calling algorithms were employed to ensure robust, reproducible peak calls. First, tag directories were created using HOMER makeTagDirectory for each replicate, and peaks were called using default parameters for findPeaks with—style factor. MACS2 was also called using default parameters on each replicate. The summit files output by MACS2 were converted to bed format and each summit extended bidirectionally to achieve a total length of 300 bp. As the ATAC-seq peak calls would ultimately be used to identify a small number of highly enriched potential regulatory elements for screening of a limited subset, the Inventors applied the overly stringent requirement that a peak be called by both approaches in a given replicate for its inclusion in the final peak list for that sample. Peaks identified in any sample in this way were aggregated to produce a final superset of 323,369 regulatory elements called as accessible in at least one cell type. The feature counts package was used to obtain ATAC-seq read counts for each of these accessible putative GREs. This approach reduced the rate of false positive peaks.

Identification of SST-enriched GREs: The Inventors used genomic coordinates of a superset of 323,369 genomic regions identified as a union of ATAC-Seq peaks across various cell types in the mouse cortex as a list of reference coordinates over which to quantify the ATAC-Seq signal from SST+, VIP+ and PV+ cells. A matrix was constructed representing the mean ATAC-Seq signal in SST+, VIP+ and PV+ cells for each of the 323,369 GREs and normalized such that the total ATAC-Seq signal from each cell population was scaled to 10⁷. Fold-enrichment was calculated for each region/GRE as [(Signal in cell type A)+1]/[mean(signal in cell types B and C)+1]. GREs were subsequently ranked based on fold-enrichment score.

Identification of conserved GREs: To identify GREs whose sequence is highly conserved across mammals, the Inventors first needed to identify an appropriate conservation score to use as a threshold for high conservation. The Inventors reasoned that by analyzing the conservation of DNA sequences of the same length, but an arbitrary distance of 100,000 bases away from each identified GRE, the Inventors would generate a set of DNA sequences whose conservation can be used to determine this threshold.

To this end, conservation scores for GREs and corresponding GRE-distal sequences were calculated using the bigWigAverageOverBed command to determine the average PhyloP score of each sequence based on mm10.60way.phyloP60wayPlacental.bw PhyloP scores (available on the world wide web at hgdownload.cse.ucsc.edu/goldenpath/mm10/phyloP60way/). After plotting the conservation score (phyloP, 60 placental mammals) of 323,369 GRE-distal sequences, the Inventors determined the conservation score of the 95^{th} percentile of this distribution (PhyloP score=0.5) and chose it as a minimal conservation score needed to classify any GRE as conserved.

Viral barcode design: Viral barcode sequences were chosen to be at least 3 insertions, deletions, or substitutions apart from each other to minimize the effects of sequencing errors on the correct identification of each barcode. The R library “DNAbarcodes” and following functions were used:

initialPool=create.dnabarcodes(10, dist=3, heuristic=“ashlock”);

finalPool=create.dnabarcodes(10, pool=initialPool, metric=“seqlev”);

The result was a list of 1164 10-base barcodes that fit the Inventors' initial criteria.

Amplification of GREs and Barcoding

Genomic PCR: PCR primers were designed using primer3 2.3.7. such that a 150-400 bp flanking sequence was added to each side of the GRE. The forward primers contained a 5′ overhang sequence for downstream in-Fusion (Clonetech™) cloning into the AAV vector (SEQ ID NO: 1—5′-GCCGCACGCGTTTAAT). The reverse primers contained a 5′ overhang sequence containing the recognition sites for AsiSI and SalI restriction enzymes (SEQ ID NO: 2—5′-GCGATCGCTTGTCGAC). Hot Start High-Fidelity Q5 polymerase (NEB™) was used according to manufacturer's protocol with mouse genomic DNA as template.

Barcoding PCR: The unpurified PCR products from the genomic PCR were used as templates for the barcoding PCR. A forward primer containing the sequence for downstream in-Fusion (Clonetech™) cloning into the AAV vector (SEQ ID NO: 3—5′-CTGCGGCCGCACGCGTTTA) was used in all reactions. Reverse primers were constructed featuring (in the 5′→3′direction): 1) a sequence for downstream in-Fusion (Clonetech™) cloning into the AAV vector (SEQ ID NO: 4—5′-GCCGCTATCACAGATCTCTCGA), 2) a unique 10-base barcode sequence, and 3) sequence complementary with the AsiSI and SalI restriction enzyme recognition sites that were introduced during the first PCR (SEQ ID NO: 5—5′-GCGATCGCTTGTCGAC). Three different reverse primers were used for each of the GREs amplified during the genomic PCR. Hot Start High-Fidelity Q5™ polymerase (NEB™) was used according to the manufacturer's protocol.

PESCA Library cloning: All PCR reactions were pooled and the amplicons purified using Agencourt AMPure XP™. The pAAV-mDlx-GFP-Fishell-1 is available from Addgene™ (plasmid #83900). The plasmid was digested with Pad and XhoI, leaving the ITRs and the polyA sequence. in-Fusion was used to shuttle the pool of GRE PCR products into the vector. Following transformation into High Efficiency NEB™ 5-alpha Competent E. coli and recovery, SalI and AsiSI were used to linearize the AAV vector containing the GREs. The expression cassette containing the human HBB promoter and intron followed by GFP and WPRE was isolated by PCR amplification from pAAV-mDlx-GFP-Fishell-1. The expression cassette was ligated with the linearized GRE-library-containing vector using T4 ligase and transformed into High Efficiency NEB™ 5-alpha Competent E. coli to yield the final library. 50 colonies were Sanger sequenced to determine the correct pairing between GRE and barcode and the correct arrangement of the AAV vector.

AAV preparation: The pooled PESCA library or individual AAV constructs (100 μg) were packed into AAV9. The titers (2-50×10¹³ genome copies/mL) were determined by qPCR. Next generation sequencing using the NextSeq 500 platform was used to determine the complexity of the pooled PESCA library (see e.g., FIG. 2A).

VI cortex injections: Animals were anesthetized with isoflurane (1-3% in air) and placed on a stereotactic instrument (Kopf™) with a 37° C. heated pad. The PESCA library (AAV9, 1.9×10¹³ genome copies/mL) was stereotactically injected in V1 (800 nL per site at 25 nL/min) using a sharp glass pipette (25-45 μm diameter) that was left in place for 5 min prior to and 10 min following injection to minimize backflow. Two injections were performed per animal at coordinates 3.0 and 3.7 mm posterior, 2.5 mm lateral relative to bregma, and 0.6 mm ventral relative to the brain surface.

Individual rAAV-GRE constructs were stereotactically injected at a titer of 1×10¹¹ genome copies/mL. (250 nL per site at 25 nL/min). All injections were performed at two depths (0.4 and 0.7 mm ventral relative to the brain surface) to achieve broader infection across cortical layers. The injection coordinates relative to bregma were 3.0 or 3.7 mm posterior, 2.5 or −2.5 mm lateral.

Nuclear isolation: Single-nuclei suspensions were generated as described previously, with minor modifications. V1 was dissected and placed into a Dounce with homogenization buffer (e.g., 0.25 M sucrose, 25 mM KCl, 5 mM MgCl₂, 20 mM Tricine-KOH, pH 7.8, 1 mM DTT, 0.15 mM spermine, 0.5 mM spermidine, protease inhibitors). The sample was homogenized using a tight pestle with 10 stokes. IGEPAL solution (5%, Sigma™) was added to a final concentration of 0.32%, and 5 additional strokes were performed. The homogenate was filtered through a 40-μm filter, and OptiPrep (Sigma™) added to a final concentration of 25% iodixanol. The sample was layered onto an iodixanol gradient and centrifuged at 10,000 g for 18 minutes as previously described^{1,2}. Nuclei were collected between the 30% and 40% iodixanol layers and diluted to 80,000-100,000 nuclei/mL for encapsulation. All buffers contained 0.15% RNasin® Plus RNase Inhibitor (Promega™) and 0.04% BSA.

snRNA-Seq library preparation and sequencing: Single nuclei were captured and barcoded whole-transcriptome libraries prepared using the inDrops™ platform as previously described, collecting five libraries of approximately 3,000 nuclei from each animal. Briefly, single nuclei along with single primer-carrying hydrogels were captured into droplets using a microfluidic platform. Each hydrogel carried oligodT primers with a unique cell-barcode. Nuclei were lysed and the cell-barcode containing primers released from the hydrogel, initiating reverse transcription and barcoding of all cDNA in each droplet. Next, the emulsions were broken and cDNA across ˜3000 nuclei pooled into the same library. The cDNA was amplified by second strand synthesis and in vitro transcription, generating an amplified RNA intermediate which was fragmented and reverse transcribed into an amplified cDNA library.

For enrichment of virally-derived transcripts, a fraction (3 μL) of the amplified RNA intermediate was reverse transcribed with random hexamers without prior fragmentation. PCR was next used to amplify virally derived transcripts. The forward primer was designed to introduce the R1 sequence and anneal to a sequence uniquely present 5′ of the viral-barcode sequence present in the viral transcripts (SEQ ID NO: 6—5′-GCATCGATACCGAGCGC). The reverse primer was designed to anneal to a sequence present 5′ of the cell-barcode (SEQ ID NO: 7—5′-GGGTGTCGGGTGCAG). The result of the PCR is preferential amplification of the viral-derived transcripts, while simultaneously retaining the cell-barcode sequence necessary to assign each transcript to a particular cell/nucleus. Following PCR amplification (18 cycles, Hot Start High-Fidelity Q5™ polymerase) all the libraries were indexed, pooled, and sequenced on a Nextseq 500™ benchtop DNA sequencer (Illumina™).

inDrop™ sample mapping and viral barcode deconvolution by cell: The published inDrops™ mapping pipeline (see e.g., available on the world wide web at github.com/indrops/indrops) was used to assign reads to cells. To map viral sequences, a custom annotated transcriptome was generated using the indrops pipeline build_index command supplied with the following newly generated reference files: a custom genome with one additional contig comprising a shared 5′ sequence (SEQ ID NO: 8-gcatcgataccgagcgcgcgatcgc), the given 10 bp barcode, and a shared 3′ sequence (SEQ ID NO: 9-tcgagagatctgtgatagcggc) was appended to the GRCm38.dna_sm.primary_assembly.fa genome file for each cloned GRE. These sequences were also appended GRCm38.88.gtf gene annotation file, with all sequences assigned the same gene_id and gene_name, but unique transcript_id, transcript_name, and protein_id. After inDrops pipeline mapping and cell deconvolution, the pysam package was used to extract the ‘XB’ and ‘XU’ tags, which contain cell barcode and UMI sequences, respectively, from every read that mapped uniquely to any one of the custom viral contigs (i.e. requiring the read map to the 10 bp barcode with at most 1 mismatch) in the inDrops pipeline-output bam files. These barcode-UMI combinations were condensed to generate a final cell×GRE barcode UMI counts table for each sample.

Embedding and identification of cell types: Data from all nuclei (two animals, 5 libraries of ˜3,000 nuclei per animal) were analyzed simultaneously. Viral-derived sequences were removed for the purposes of embedding clustering and cell type identification. The initial dataset contained 32,335 nuclei, with more than 200 unique non-viral transcripts (UMIs) assigned to each nucleus. The R software package Seurat was used to cluster cells. First, the data were log-normalized and scaled to 10,000 transcripts per cell. Variable genes were identified using the FindVariableGenes( ) function. The following parameters were used to set the minimum and maximum average expression and the minimum dispersion: x.low.cutoff=0.0125, x.high.cutoff=3, y.cutoff=0.5. Next, the data was scaled using the ScaleData( ) function, and principle component analysis (PCA) was carried out. The FindClusters( ) function using the top 30 principal components (PCs) and a resolution of 1.5 was used to determine the initial 29 clusters. Based on the expression of known marker genes the Inventors merged clusters that represented the same cell type. The Inventors' final list of cell types was: Excitatory neurons, PV Interneurons, SST Interneurons, VIP interneurons, NPY Interneurons, Astrocytes, Vascular-associated cells, Microglia, Oligodendrocytes, and Oligodendrocyte precursor cells.

Enrichment calculation: Viral vector expression for each of the 861 barcodes across the ten cell types was calculated by averaging the expression of barcoded transcripts across all the individual nuclei that were assigned to that cell type. The relative fold-enrichment in expression toward Sst+ cells was computed as the ratio of the mean expression in Sst+ cells and the mean expression in Sst− cells: (mean(Sst+ cells)+0.01)/(mean(Sst− cells)+0.01).

Viral GRE expression for each of the 287 barcodes was calculated at the single-nucleus level as a sum of the expression of the three barcodes that were paired with that GRE. Average GRE-driven expression across the ten cell types was calculated by averaging the expression of the GRE transcripts across all the individual nuclei that were assigned to that cell type. The relative fold-enrichment in GRE expression toward Sst+ cells was determined as the ratio of the mean expression in Sst+ cells and the mean expression in Sst− cells: (mean(Sst+ cells)+0.01)/(mean(Sst− cells)+0.01).

Differential gene expression: To identify which of the GRE-driven transcripts were statistically enriched in Sst+vs. Sst− cells, the Inventors carried out differential gene expression analysis using the R package Monocle2. The data were modeled and normalized using a negative binomial distribution, consistent with snRNA-seq experiments. The functions estimateSizeFactors( ) estimateDispersions( ) and differentialGeneTest( ) were used to identify which of the GRE-derived transcripts were statistically enriched in Sst+ cells. GREs whose false discovery rate (FDR) was less than 0.01 were considered enriched.

Fluorescence microscopy, Sample preparation: Mice were sacrificed and perfused with 4% PFA followed by PBS. The brain was dissected out of the skull and post-fixed with 4% PFA for 1-3 days at 4° C. The brain was mounted on the vibratome (Leica™ VT1000S) and coronally sectioned into 100 μm slices. Sections containing V1 were arrayed on glass slides and mounted using DAPI Fluoromount-G (Southern Biotech™).

Sample imaging: Sections containing V1 were imaged on a Leica™ SPE confocal microscope using an ACS APO 10×/0.30 CS objective. Tiled V1 cortical areas of ˜1.2 mm by ˜0.5 mm were imaged at a single optical section to avoid counting the same cell across multiple optical sections. Channels were imaged sequentially to avoid any optical crosstalk.

Immunostaining: To identify parvalbumin (PV)+ cells, coronal sections were washed three times with PBS containing 0 3% TritonX-100 (PBST) and blocked for 1 h at room temperature with PBST containing 5% donkey serum. Section were incubated overnight at 4° C. with mouse anti-PVALB antibody 1:2000 (Millipore™), washed again three times with PBST, and incubated for 1 h at room temperature with 1:500 donkey anti-mouse 647 secondary antibody (Life Technologies™). After washing in PBST and PBS, samples were mounted onto glass slides using DAPI Fluoromount-G.

Quantification of the percentage of GFP+ cells that were SST+, VIP+, and PV+: Across all images, coordinates were registered for each GFP+cell that could be visually discerned. An automated ImageJ script was developed to quantify the intensity of each acquired channel for a given GFP+cell. The Inventors created a circular mask (radius=5.7 μm) at each coordinate representing a GFP positive cell, background subtracted (rolling ball, radius=72 μm) each channel, and quantified the mean signal of the masked area. To identify the threshold intensity used to classify each GFP+cell as either SST+, VIP+ or PV+, the Inventors first determined the background signal in the channel representing SST, VIP or PV by selecting multiple points throughout the area visually identified as background. These background points were masked as small circular areas (radius=5.7 μm), over which the mean background signal was quantified. The highest mean background signal for SST, VIP and PV was conservatively chosen as the threshold for classifying GFP+ cells as SST+, VIP+ or PV+, respectively.

Quantification of the distribution of cells as a function of distance from pia: A semiautomated ImageJ™ algorithm was developed to trace the pia in each image, generate a Euclidean Distance Map (EDM), and calculate the distance from the pia to each GFP+cell.

Quantification of the percentage of SST+ cells that were GFP+: An automated algorithm was developed to identify SST+ cells after appropriate background subtraction, image thresholding, masking and filtering for all objects of appropriate size and circularity. The number of SST+objects (cells) was then counted within a minimal polygonal area that encompassed all GFP+ cells in that image. The ratio of the number of GFP+ cells and SST+ cells within the area of infection (here identified as area with discernable GFP+ cells) was calculated.

Slice Preparation: Acute, coronal brain slices containing visual cortex of 250-300 μm thickness were prepared using a sapphire blade (Delaware Diamond Knives™) and a VT1000S vibratome (Leica™). Mice were anesthetized though inhalation of isoflurane, then decapitated. The head was immediately immersed in an ice-cold solution containing (in mM): 130 K-gluconate, 15 KCl, 0.05 EGTA, 20 HEPES, and 25 glucose (pH 7.4 with NaOH; Sigma™). The brains were quickly dissected and cut in the same ice-cold, gluconate based solution while oxygenated with 95% O₂/5% CO₂. Slices then recovered at 32° C. for 20-30 minutes in oxygenated artificial cerebrospinal fluid (ACSF) in mM: 125 NaCl, 26 NaHCO₃, 1.25 NaH2PO4, 2.5 KCl, 1.0 MgCl2, 2.0 CaCl2, and 25 glucose (Sigma), adjusted to 310-312 mOsm with water.

Electrophysiological Recordings: Whole-cell current clamp recordings of fluorescent, DREADD-expressing neurons in coronal visual cortex slices of P50 to P80 wild-type mice were performed using borosilicate glass pipettes (3-5 MOhms, Sutter Instrument™) filled with an internal solution (in mM): 116 KMeSO3, 6 KCl, 2 NaCl, 0.5 EGTA, 20 HEPES, 4 MgATP, 0.3 NaGTP, 10 NaPO₄ creatine (pH 7.25 with KOH; Sigma™). All experiments were performed at room temperature in oxygenated ACSF. Series resistance was compensated by at least 60%. After break-in, a systematic series of 1 second current injections ranging from −100 pA to 500 pA were applied to each cell using the User List function in the “Edit Waveform” tab of pClamp. After such baseline firing rates were calculated, CNO (2 μM, Sigma) was bath applied. An average of at least three trials for each current injection was calculated before and during CNO application.

Data Acquisition and Analysis: For electrophysiology, data acquisition of current-clamp experiments was performed using Clampex10.2™, an Axopatch 200B™ amplifier, and digitized with a DigiData 1440™ data acquisition board (Molecular Devices™). Analysis of firing rate and membrane potential was done using Clampfit™ (Molecular Devices™) and Prism7™ (GraphPad Software™).

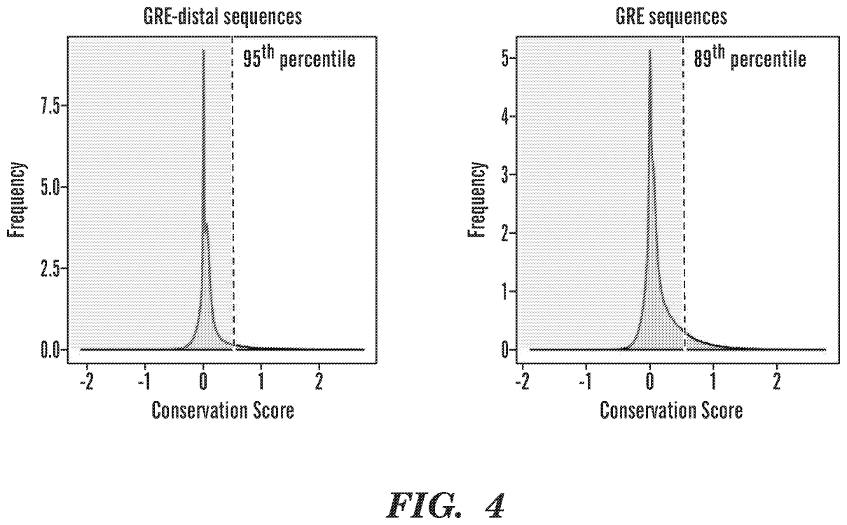

GRE selection and library construction: To identify candidate SST interneuron-restricted gene regulatory elements (GREs), the Inventors carried out comparative epigenetic profiling of the three largest classes of cortical interneurons, somatostatin (SST)−, vasoactive intestinal polypeptide (VIP)- and parvalbumin (PV)-expressing cells. To this end, the Inventors employed the recently developed isolation of nuclei tagged in specific cell types (INTACT) method to isolate purified chromatin from of each of these cell types from the cerebral cortex of adult (6-10-week-old) mice. Assay for transposase-accessible chromatin using sequencing (ATAC-Seq), which marks nucleosome-depleted gene regulatory regions based on their enhanced accessibility to in vitro transposition by the Tn5 transposase, was then used to identify genomic regions with enhanced accessibility in the SST (n=279,221), PV (n=275,631), and VIP (n=258,646) chromatin samples. Among these putative gene regulatory regions, 16,386 (5.9%) were enriched or uniquely present in SST cells (see e.g., FIG. 1B, FIG. 1C). To enrich for GREs that might function across mammalian species, the Inventors subsequently filtered the resulting list to exclude GREs with poor mammalian sequence conservation (see e.g., Experimental Methods, FIG. 4). Remaining elements were ranked based on cell-type-specificity (see e.g., Experimental Methods), with the top 287 SST-enriched GREs selected for screening (see e.g., FIG. 1D, Table 3).

A PCR-based strategy was used to simultaneously amplify and barcode each GRE from mouse genomic DNA (see e.g., Experimental Methods). To minimize sequencing bias due to the choice of barcode sequence, each GRE was paired with three unique barcode sequences. The resulting library of 861 GRE-barcode pairs was pooled and cloned into an AAV-based expression vector, with the GRE element inserted 5′ to a minimal promoter driving a GFP expression cassette and the GRE-paired barcode sequences inserted into the 3′ untranslated region (UTR) of the GRE-driven transcript (see e.g., Experimental Methods, FIG. 2A, FIG. 5). This configuration was chosen to maximize the retrieval of the barcode sequence during single-cell RNA sequencing. The library was packaged into AAV9, which exhibits broad neural tropism. The complexity of the resulting rAAV-GRE library was then confirmed by Next Generation Sequencing, detecting 802 of the 861 barcodes (93.2%), corresponding to 285 of the 287 GREs (99.3%) (see e.g., FIG. 2B).

PESCA Screening

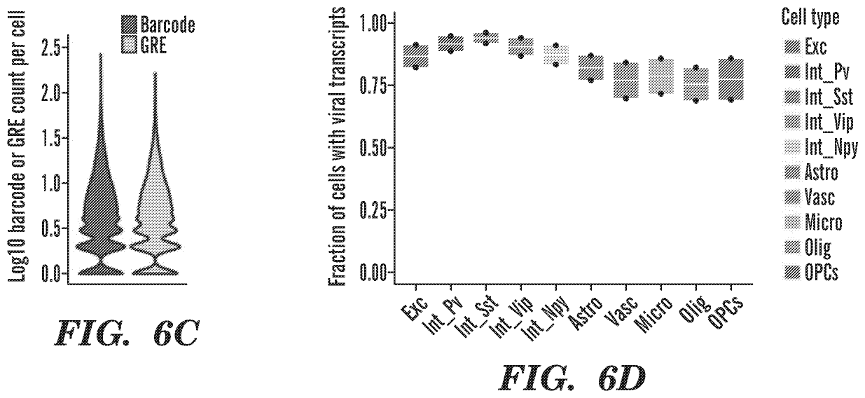

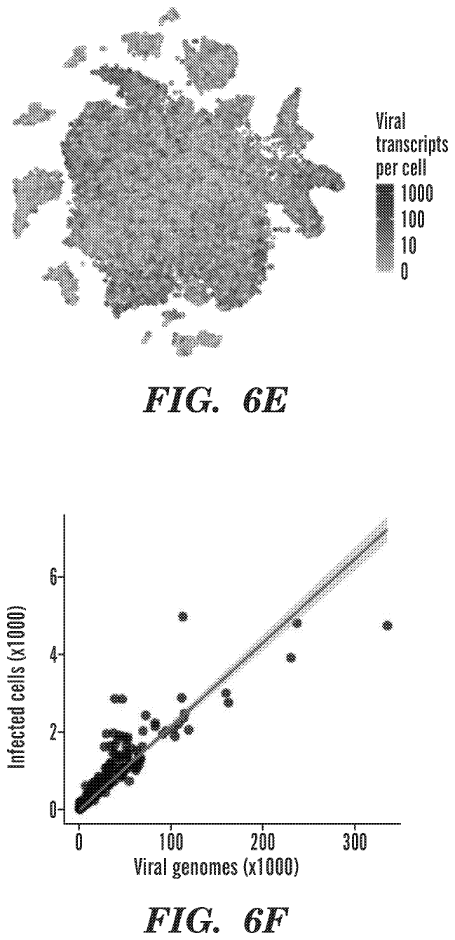

To quantify the expression of each rAAV-GRE vector across the full complement of cell types in the mouse visual cortex, the Inventors used a modified single-nucleus RNA-Seq (snRNA-Seq) protocol to first determine the cellular identity of each nucleus and then quantify the abundance of the GRE-paired barcodes in the transcriptome of nuclei assigned to each cell type. Two injections (800 nL each) of the pooled AAV library (1×10¹³ viral genomes/mL) were first administered to the primary visual cortex (V1) of two 6-week-old C57BL/6 mice. Twelve days following injection, the injected cortical regions were dissected and processed to generate a suspension of nuclei for snRNA-Seq using the inDrops™ platform. A total of 32,335 nuclei were subsequently analyzed across the two animals, recovering an average of 866 unique non-viral transcripts per nucleus, representing 610 unique genes (see e.g., FIG. 6A-6B).

Since droplet-based high-throughput snRNA-Seq samples the nuclear transcriptome with low sensitivity, viral-derived transcripts were initially detected in only 3.9% of sampled nuclei. The Inventors therefore designed a modified PCR-based approach to enrich for barcode-containing viral transcripts, which yielded deep coverage of AAV-derived transcripts with simultaneous shallow coverage of the non-viral transcriptome. PCR enrichment increased the viral transcript recovery 382-fold in the sampled nuclei, to an average of 15.6 unique viral transcripts, 6.0 unique GRE-barcodes, and 5.7 unique GREs per cell (see e.g., FIG. 2B, FIG. 6C). Using this modified protocol, viral transcripts were identified across 86% of cells (see e.g., FIG. 6D-6E), with a high correlation (r=0.9, p<2.2×10⁻¹⁶) observed between the abundance of each barcoded AAV in the library and the number of cells infected by that AAV (see e.g., FIG. 6F).

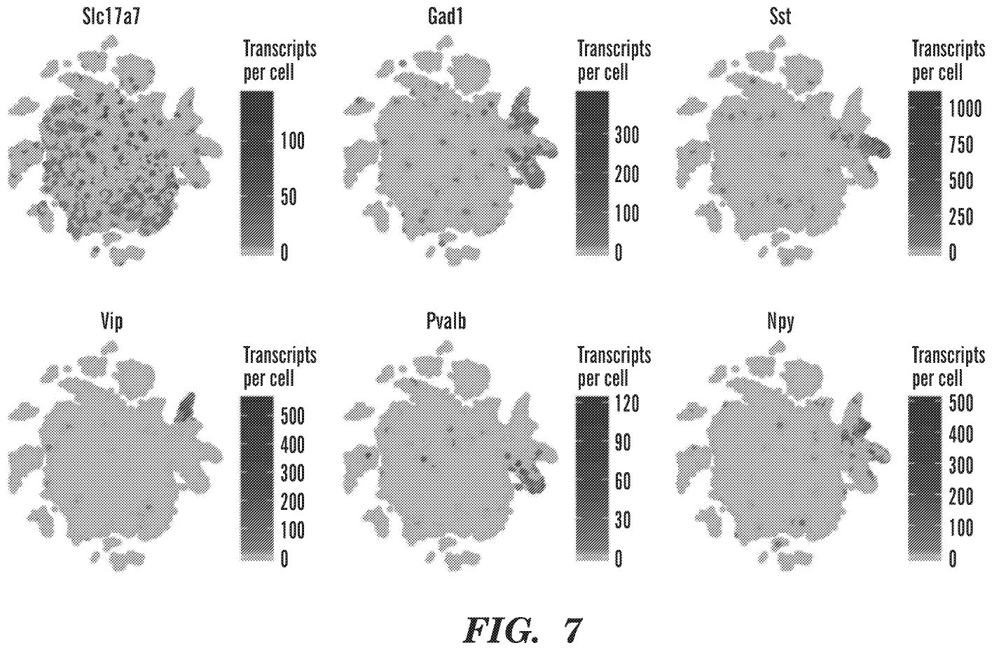

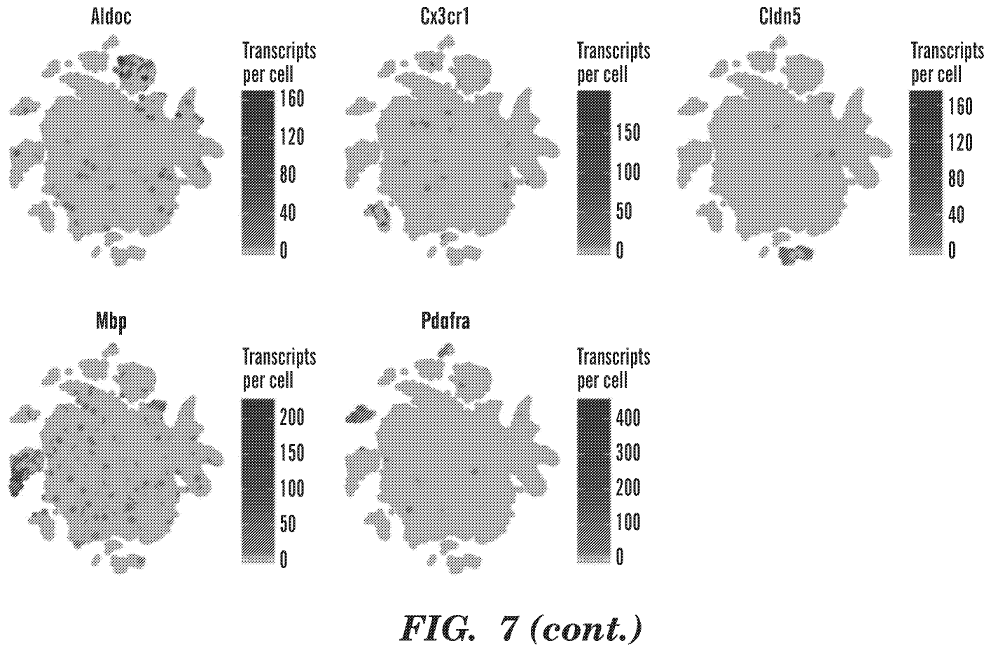

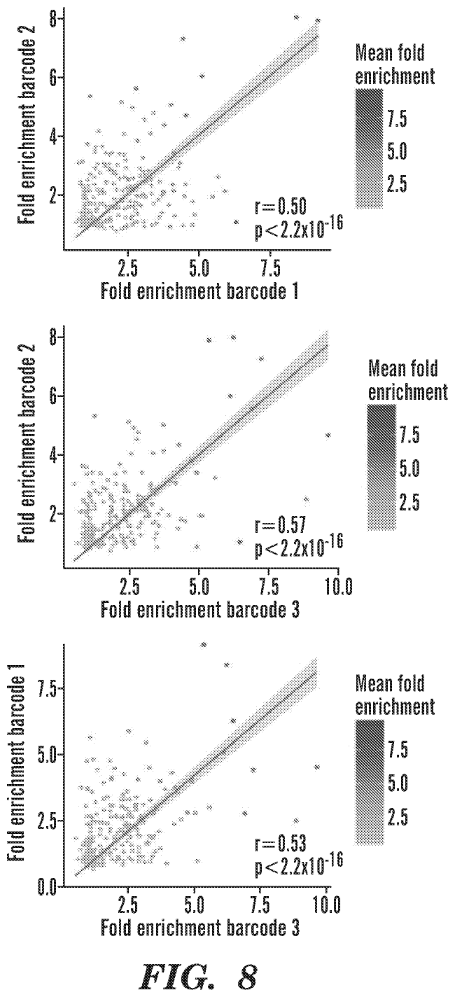

Nuclei were classified into 10 cell types using graph-based clustering and expression of known marker genes (see e.g., Experimental Methods, FIG. 2C-2D, FIG. 7). The average expression of each viral-derived barcoded transcript was analyzed across all cell types, and an enrichment score was calculated from the ratio of expression in Sst⁺ nuclei compared to all Ssf nuclei. As expected, sets of three barcodes associated with the same GRE showed highly statistically correlated enrichment scores (r=0.53±0.03, p<2.2×10⁻¹⁶) (see e.g., FIG. 2E-2F, FIG. 8), which were abolished when barcodes were randomly shuffled (shuffled r=0.002±0.06; Wilcox test between data and shuffled data, p=0.003).

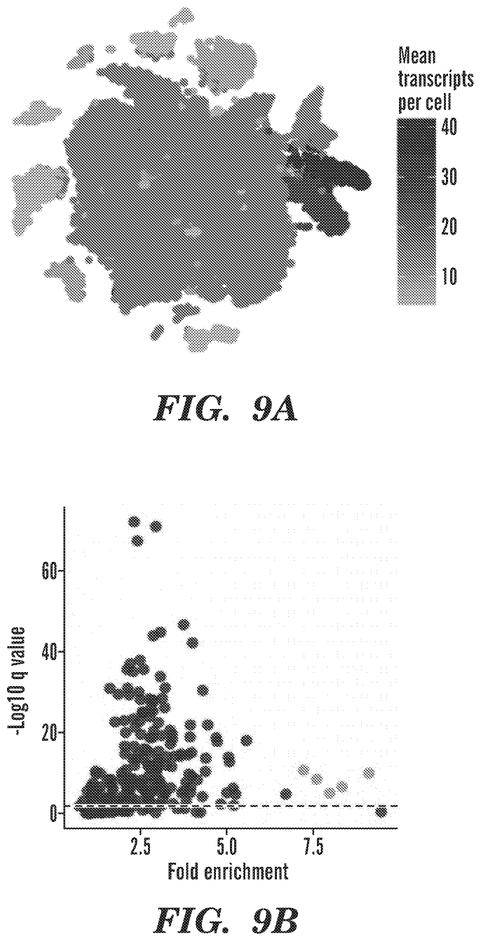

Having confirmed a robust, non-random correlation in enrichment scores among the three barcodes associated with each GRE, the Inventors next computed a single expression value for each of the 287 viral drivers by aggregating expression data from barcodes associated with the same GRE, and carried out differential gene expression analysis between Sst⁺ and Ssf cells for each rAAV-GRE. Differential gene expression analysis between Sst⁺ and Ssf cells for each rAAV-GRE revealed a marked overall enrichment of viral-derived transcripts in the Sst⁺ subpopulation (see e.g., FIG. 9A). Indeed, multiple viral drivers were identified that promoted highly specific reporter expression in the Sst⁺ subpopulation (q<0.01, fold-change>7; see e.g., FIG. 2G-2I, FIG. 9B).

In Situ Characterization of rAAV-GRE Reporter Expression

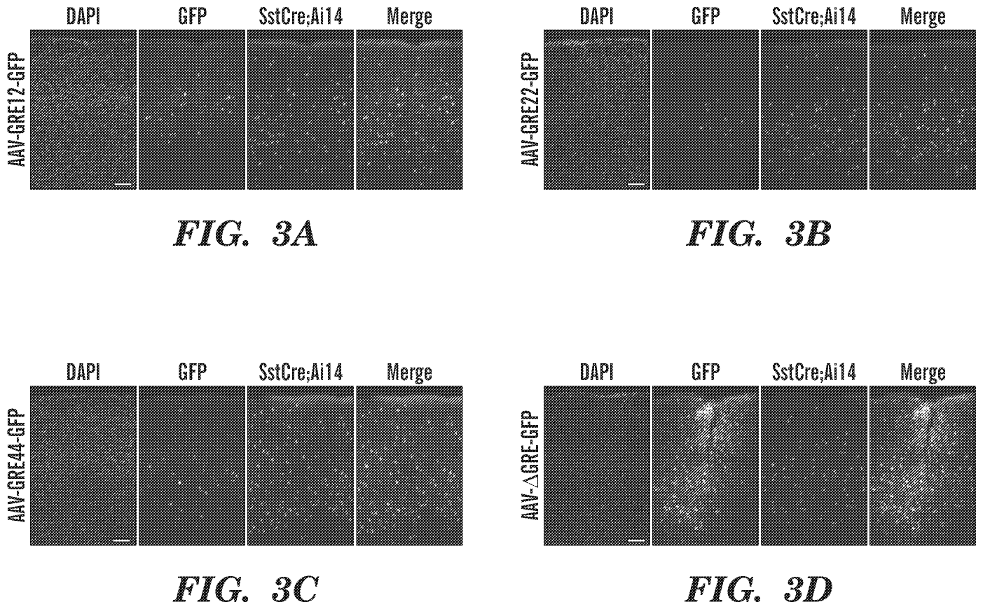

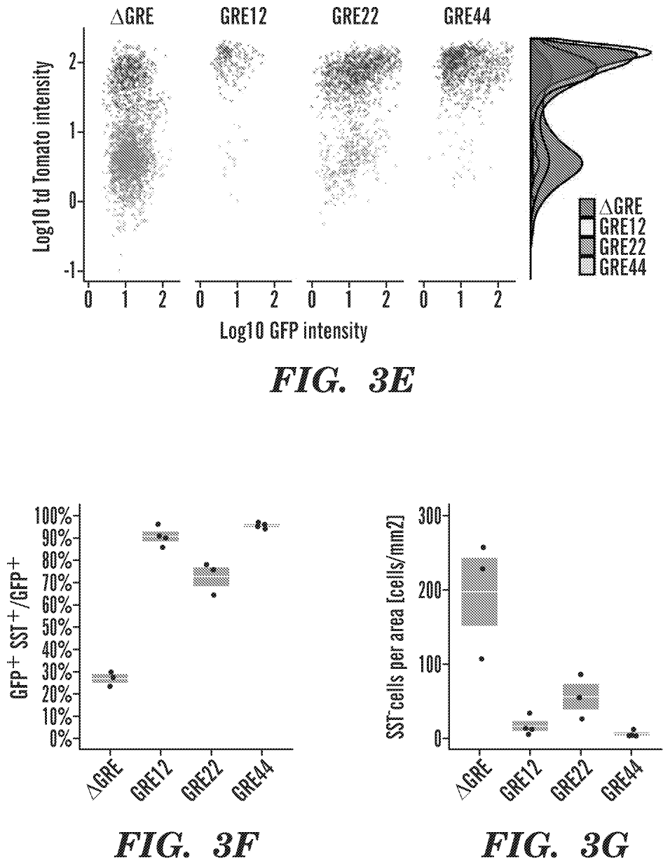

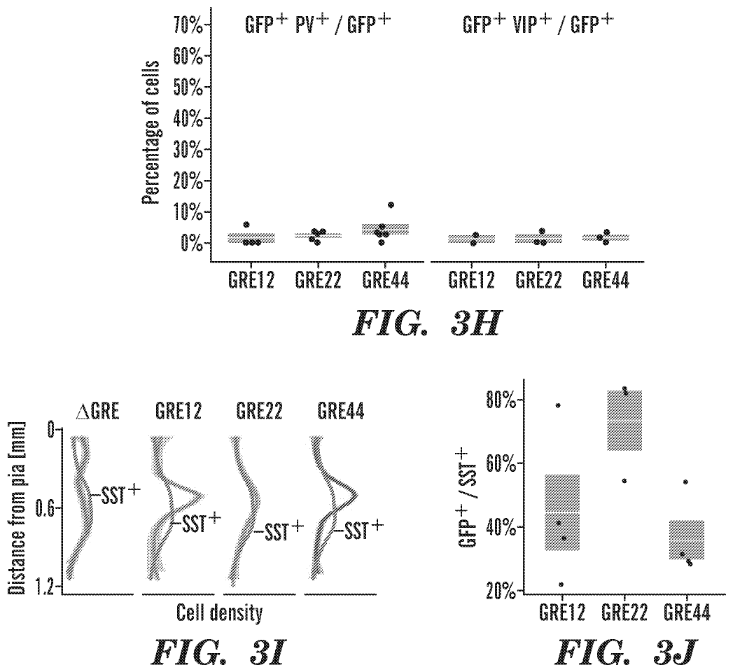

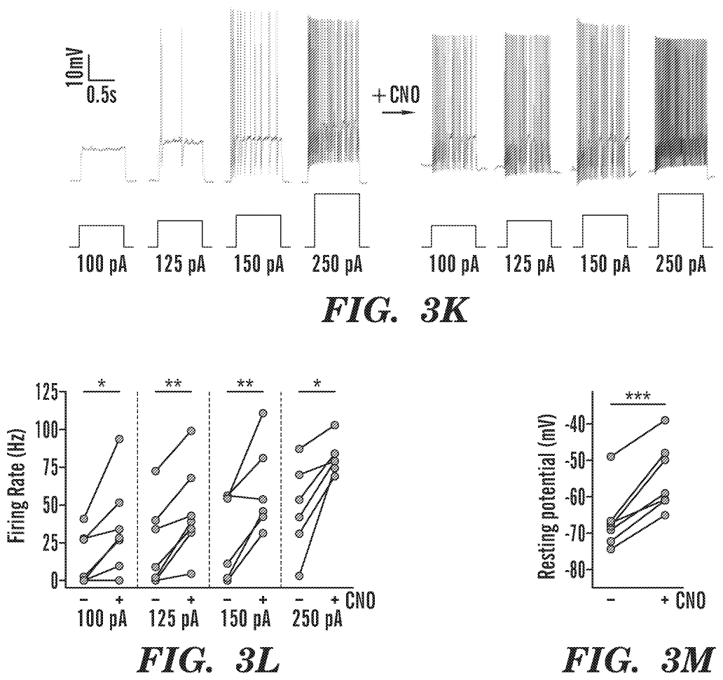

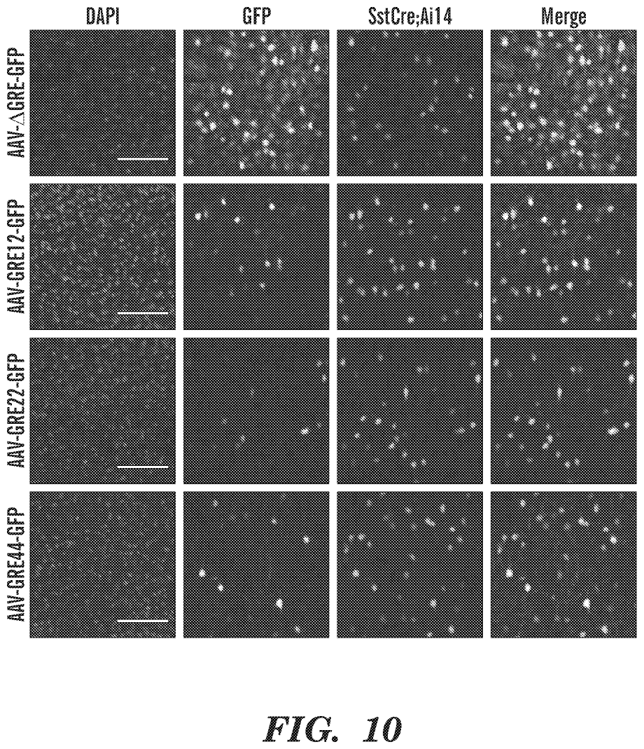

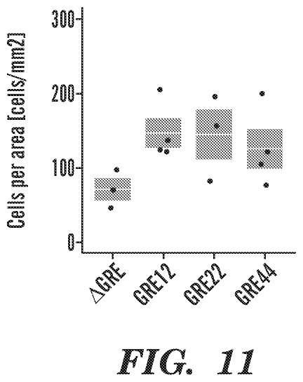

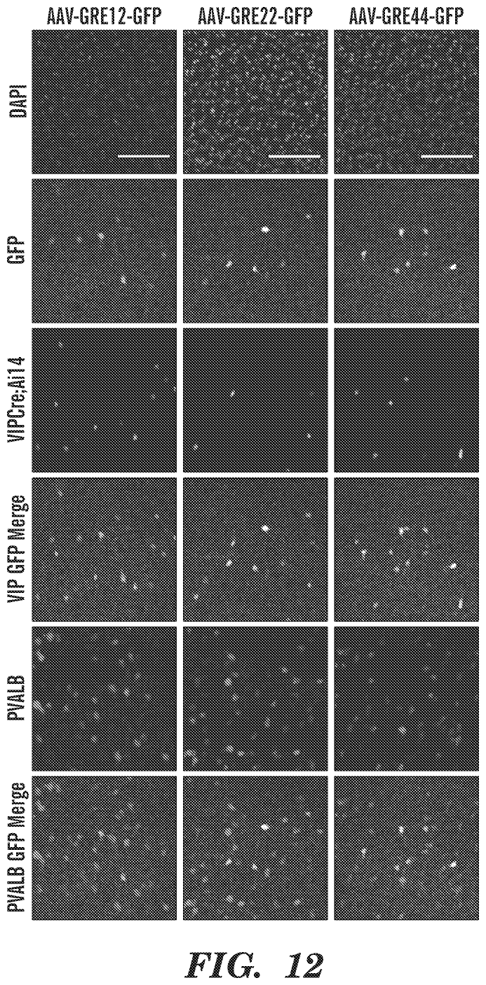

The Inventors next sought to validate the cell-type-specificity of the resulting hits using methods that do not rely on single-cell sequencing-based approaches. To this end, the Inventors selected three of the top five viral drivers (GRE12, GRE22, GRE44), as well as a control viral construct lacking the GRE element (ΔGRE), for injection into V1 of adult transgenic Sst-Cre; Ai14 mice, in which SST⁺ cells express the red fluorescent marker tdTomato. Fluorescence analysis twelve days following injection with rAAV-GRE12/22/44-GFP revealed strong yet sparse GFP labeling centered around cortical layers IV and V (see e.g., FIG. 3A-3C). By contrast, the control rAAV-ΔGRE-GFP showed a strikingly different pattern of GFP expression concentrated around the sites of injection, with expression in a larger number of cells (see e.g., FIG. 3D). Many virally infected cells were indeed SST-positive, marked by the high degree of overlapping GFP and tdTomato expression: 90.7%±2.1% for rAAV-GRE12-GFP (170 cells, 4 animals); 72.9±4.2% for rAAV-GRE22-GFP (1164 cells, 3 animals), and 95.8±0.6% for rAAV-GRE44-GFP (759 cells, 4 animals) (see e.g., FIG. 3E-3F, FIG. 10). By contrast, the Inventors observed that only 27.2±1.9% of GFP⁺ cells following rAAV-ΔGRE-GFP infection were also positive for tdTomato expression (2066 cells, 3 animals; see e.g., FIG. 3E-3F), indicating that the tested GREs serve to effectively restrict AAV payload expression to SST⁺ interneurons. It is notable that the GREs seemingly not only promote expression in SST⁺ cells but also reduce background expression in SST⁻ cells, indicating the tested GREs confer both enhancer and insulator functionality. Consistent with his hypothesis, the incorporation of the GREs into the rAAV both increased the number of SST⁺/GFP⁺ cells (1.7-2-fold) and dramatically (3-32-fold) decreased the number of SST⁻ cells that expressed GFP (see e.g., FIG. 3G, FIG. 11). To further investigate the specificity of the Inventors' viral drivers among cortical interneuronal cell types the Inventors injected each construct into Vip-Cre; Ai14⁺ mice in which all VIP⁺ cells express tdTomato, or used fluorescence antibody staining to label PV-expressing cells (see e.g., FIG. 12). Fluorescent signal analysis indicated the percentage of GFP⁺ cells that were either VIP⁺ or PV⁺ (rAAV-SST12-GFP⁺ [2.6±2.6%], rAAV-GRE22-GFP⁺ [3.5±2.0%] and rAAV-GRE44-GFP⁺ [6.0±2.7%]; see e.g., FIG. 3H). This confirms that among major interneuronal cell classes, all three vectors are highly SST-specific.

Because at least five subtypes of cortical SST⁺ interneurons have been identified based on the laminar distribution of their cell bodies and projections, the Inventors also investigated the laminar distribution of GFP-expressing cells for the three Sst-enriched viral drivers. Intriguingly, the majority of rAAV-GRE12-GFP⁺ and rAAV-GRE44-GFP⁺ SST⁺ cells were found to reside in layers IV and V, distinct from the distribution observed for the full SST⁺ cell population in visual cortex (p=1.3×10⁻⁶, p<2.2×10⁻¹⁶, respectively, Mann-Whitney U test, two-sided; see e.g., FIG. 3I), raising the possibility that these constructs may preferentially label a specific subtype(s) of SST⁺ interneuron. Consistent with this hypothesis, the Inventors observed that these two viral drivers mediated reporter expression in only a relatively small fraction of all SST⁺ cells within the region of infection (44.5±12.0% for rAAV-GRE12-GFP and 35.9±6.2% for rAAV-GRE44-GFP) compared to rAAV-GRE22-GFP (see e.g., FIG. 3J). Together, these findings suggest that PESCA may support the isolation of viral drivers capable of discriminating between fine-grained cell-types within a given interneuron cell class.

Modulation of Neuronal Activity with rAAV-GREs

Finally, the Inventors evaluated whether the identified viral drivers support sufficiently high and persistent levels of payload expression to effectively modulate SST⁺ cell physiology. Designer receptors exclusively activated by designer drugs (DREADDs) are commonly employed viral payload to dynamically regulate neuronal activity in response to the synthetic ligand clozapine-N₄-oxide (CNO). The Inventors therefore injected the visual cortex of adult mice (6-8-week-old) with rAAV-GRE12-Gq-DREADD-tdTomato (see e.g., SEQ ID NO: 22) and performed electrophysiological recordings from tdTomato⁺ cells of acute cortical slices in a whole-cell, current-clamp configuration two weeks post-injection. All recordings from tdTomato⁺ cells evoked with depolarizing current steps showed striking sensitivity to CNO, as shown by significantly increased firing rates and depolarized resting membrane potentials during bath application of CNO (see e.g., FIG. 3K-3M). These data demonstrate the ability of these reagents to robustly modulate the activity of SST⁺ cells in non-transgenic animals.

The PESCA platform merges the principle of massively paralleled reporter assays (MPRA) with scRNA-seq and represents a significant advancement in current approaches to viral vector design, as it enables the rapid screening of hundreds of viral permutations for enhanced cell-type-specificity. In this study, the Inventors applied PESCA to screen putative enhancer elements for drivers that robustly and specifically target a rare SST⁺ population of GABAergic interneurons in the mouse central nervous system, but this approach could be readily applied in diverse model organisms, tissues, and viral types. Moreover, PESCA is not limited to GRE screening; the method can be easily adapted to assess the cell-type-specificity of viral capsid variants. This study therefore demonstrates the broad utility of the PESCA platform for generating new cell-type-specific viral vectors, with important implications for both basic science and therapeutic applications.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions and methods related to GREs, constructs incorporating such GREs, methods and compositions related to identification and use of the aforementioned compositions, techniques, compositions and use of cells, solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### Example 2

The Promise of Gene Therapy

Gene therapy is a new and a rapidly growing field of medicine that can treat and even cure diseases by using viruses to add, remove or correct genes that are the underlying cause of disease. Many have for years been working on realizing the promise of gene therapy, using viral vectors. Viral vectors take advantage of evolved mechanisms that viruses employ to deliver genetic material to target cells. Viruses are biological nanoparticles.

Gene therapy can treat or cure genetic disorders, including tissue or cell-type-specific disorders (see e.g., Table 1 for non-limiting examples of such disorders). Individual genetic disorders are rare but are common in aggregate. In a full service pediatric inpatient facility, >⅔ of admissions and 80% of charges are attributable to disease with a recognized genetic component (50 million out of 62 million).

TABLE 1
Non-limiting examples of disorders that can
be treated or cured using gene therapy

| Genetic, tissue, or cell- | |
|---|---|
| specific disorders | Affected populations world-wide |
| Congenital deafness | ~7,500,000; ~1000 newborn per year |
| ALS (Lou Gehrig's disease) | ~500,000; incidence 2/100000 |
| Cystic fibrosis | ~70,000 |
| Congenital bleeding disorders | 1/1000 births |
| Congenital blindness | Congenital Blindness - 5/10000 births |
| Other forms of blindness | 3M people in the USA |
| Muscular dystrophies | Muscular dystrophies - 1/7000 births |
| Alpha-1 antitrypsin deficiency | 1/2000 people |
| Lysosomal storage disorders | 1/5000 births |
| Huntington disease | 5/10000 people |
| Rett syndrome | 1/10000 |
| Cardiovascular disease | >17,900,000 deaths/year |
| Osteoarthritis | >50,000,000 |
| Macular degeneration | >50,000,000 |
| Alzheimer's disease | ~20M-45M |
| Cancer | ~18,000,000 |
| Parkinson's disease | ~10,000,000 |
| Chronic pain | 1/10 of the population |

Recently adenovirus-associated viruses (AAVs) have emerged as a favored vehicle for delivery. AAVs do not integrate into genome, thus eliminating DNA damage and unpredictable deleterious effects that hindered initial gene therapy clinical trials. Recombinant adeno-associated virus can be used as a therapeutic vector, especially since it is relatively non-inflammatory and non-pathogenic, as well as safe and durable in non-replicative cells.

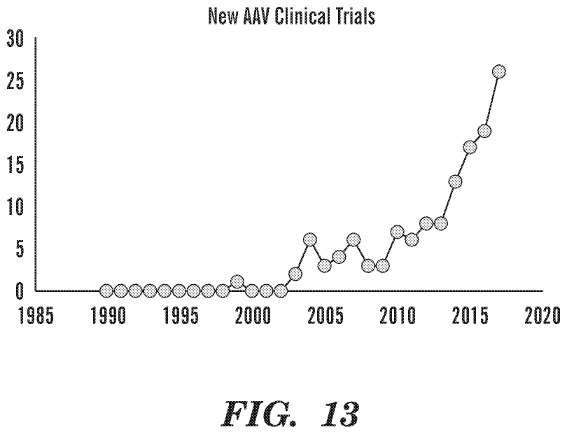

The number of clinical trials using AAVs is rapidly growing with 2018 projected to have as many new trials as all the prior years combined (see e.g., FIG. 13). For example, in late 2018, there were 174 ongoing trials, with 6 started in the most recently reported 30 day-period. These clinical trial are targeting many conditions ranging from congenital disorders to degenerative diseases. Several phase I and I/II clinical trials using AAVs have demonstrated safety and long-term (>5 years) improvement in hemophilia B or in retinal function for Leber's congenital amaurosis.

One major problem with AAV-based gene therapies is that first generation AAV vectors lack specificity. AAVs currently entering trials have not been optimized or engineered to target specific organs or cells. Therefore, these AAVs are unable to therapeutically access many tissues; they can cause significant side-effects, inflammation, and toxicity; and payload expression is often below therapeutically useful ranges. For example, as much as 90% of AAV can go to liver, leading to liver toxicity. Therefore, high viral doses are needed to achieve efficacy at the cost of significant off-target and side-effects.

The solution is to develop next generation cell-type-specific AAVs that are engineered to infect and be active only in the desired tissue. Such AAVs higher potency, higher safety, tunable and/or inducible expression, and are indisputably the future gold standard for all AAV gene therapy.

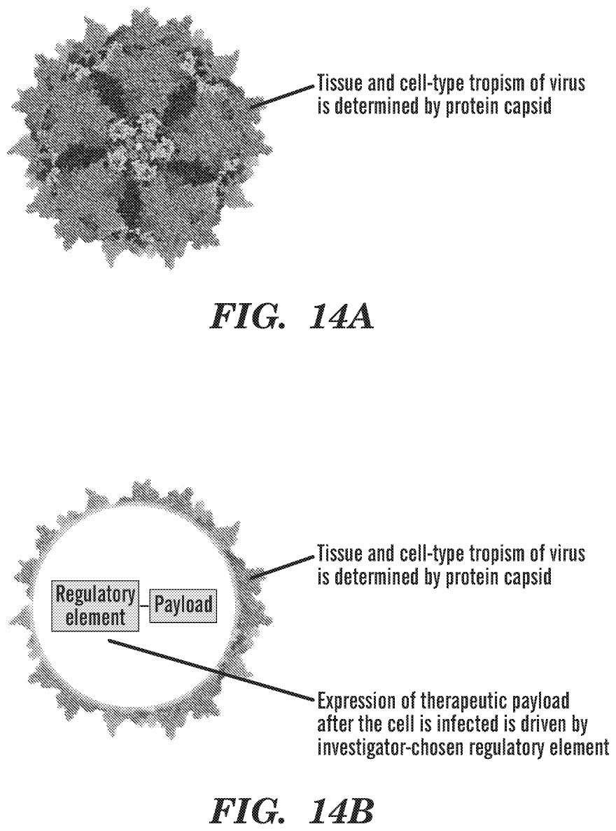

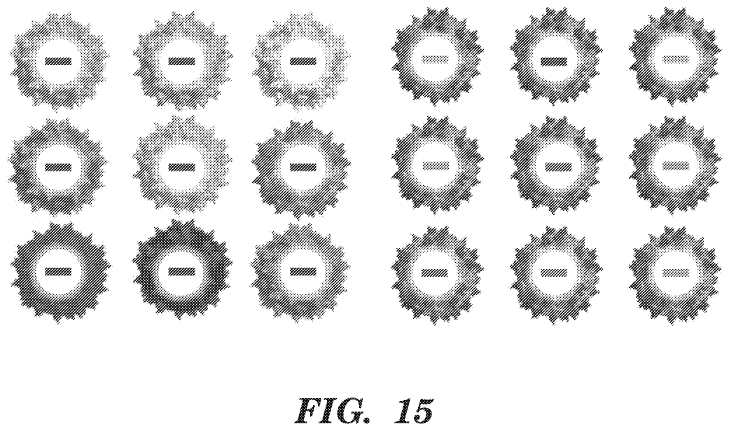

There are two approaches to engineering specificity in AAV: capsid engineering and expression engineering. The capsid (i.e., the protein shell of a virus) determines tropism and immune response (see e.g., FIG. 14A, FIG. 15). Capsid engineering is highly limited by the presence of cell-type-specific receptors necessary to take up the virus, making it previously doubtful that such a strategy would be effective. In addition, capsid efficiency and tropism varies drastically across species, and capsid engineering is a crowded area of investigation.

In expression engineering, the goal is to identify the combination of gene regulatory elements that is sufficient to drive cell-type-specific AAV expression (see e.g., FIG. 14B, FIG. 15). There is focus by others on promoter sequences, not enhancers. Current approaches to screen regulatory elements are low-throughput and not scalable. Some use machine learning to examine cell-type-specific gene expression to find promoters. Others use pre-existing databases of cell type specific promoters. Another strategy uses “promoter selection,” but all viruses currently in clinical trials use default promoters like CAG. Current AAV clinical trials all employ historically chosen promoters that confer no specificity and may not maximize payload expression and/or efficacy.

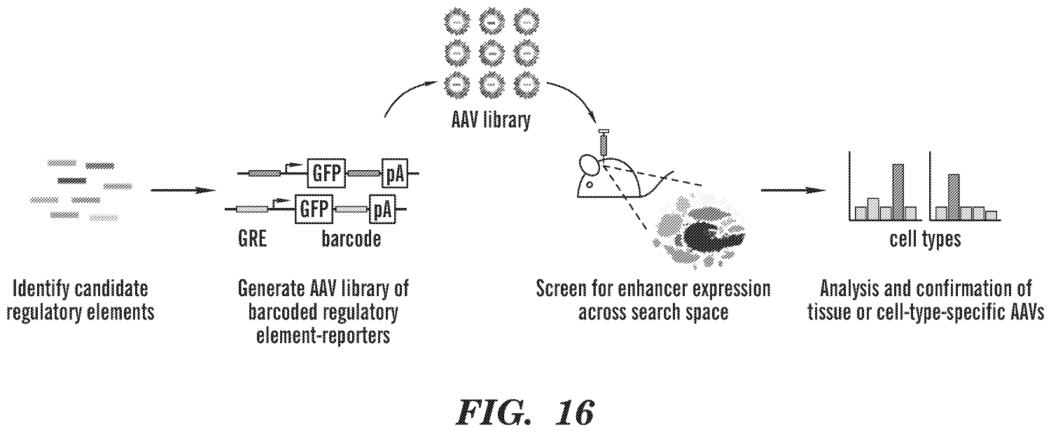

Described herein is the rapid development of tissue and cell-type-specific AAVs. The platform comprises the following steps: 1. Directly identify candidate regulatory elements using pre-existing or rapidly compiled data; 2. Generate library of AAV variants; and 3. Screen regulatory elements for cell-type or tissue-specific expression (see e.g., FIG. 16).

Driven initially by the interest to target individual cell types in the brain, the developed platform allows one to rapidly generate cell-type-specific AAVs. Briefly, to start thousands of AAV variants are generated which vary in the DNA sequence that drives the payload expression. Then in a single experiment the specificity of all of the AAVs are tested in the tissue of interest using a new single-cell sequencing platform that permits the quantification of the levels of each virus across 10,000s of individual cells in the tissue.

Instead of testing one virus at a time using fluorescence microscopy, the microscope is replaced with a sequencing technology so one can evaluate 100s or 1000s of AAVs simultaneously, and develop target-specific viruses within only a few months. This is the first platform of its kind, and it can easily be applied to a variety of tissues.

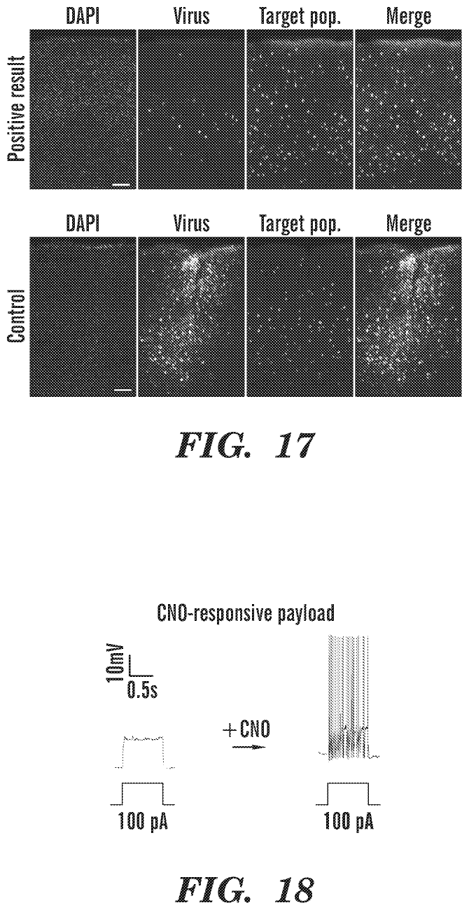

In a proof of principle study, initial tests were started with a virus with <10% on-target expression in of a rare interneuronal subtype in the brain, and from this virus a variant was developed with >90% specificity for the rare brain cell type (see e.g., FIG. 17, FIG. 18). The platform can be used to develop viruses to target other cells types in the brain as well as the retina and the inner ear.

Many advantages are conferred by the expression engineering described herein. Higher and more specific expression significantly lowers required AAV titers, increasing safety and reducing cost. Furthermore, expression engineering is a complementary approach to capsid engineering, which can both be used to generate ideal AAV vectors for gene therapy.

Finally, the platform is fast and generalizable to any target cell-type or tissue, and the platform can be directly applied in non-human primates or human cells.

### Example 3

A Scalable Platform for the Development of Cell-Type-Specific Viral Drivers

Enhancers are the primary DNA regulatory elements that confer cell type specificity of gene expression. Recent studies characterizing individual enhancers have revealed their potential to direct heterologous gene expression in a highly cell-type-specific manner. However, it has not yet been possible to systematically identify and test the function of enhancers for each of the many cell types in an organism. Described herein is PESCA, a scalable and generalizable method that leverages ATAC- and single-cell RNA-sequencing protocols, to characterize cell-type-specific enhancers that permits genetic access and perturbation of gene function across mammalian cell types. Focusing on the highly heterogeneous mammalian cerebral cortex, PESCA was applied to find enhancers and generate viral reagents capable of accessing and manipulating a subset of somatostatin-expressing cortical interneurons with high specificity. This study demonstrates the utility of this platform for developing new cell-type-specific viral reagents, with significant implications for both basic and translational research.

Enhancers are DNA elements that regulate gene expression to produce the unique complement of proteins necessary to establish a specialized function for each cell type in an organism. Large scale efforts to build a definitive catalog of cell based on their gene expression have successfully mapped epigenomic regulatory landscapes, permitting a mechanistic understanding of the underlying gene expression that is critical for cell-type-specific development, identity, and unique function. Importantly, characterization of individual enhancers has revealed their potential to direct highly cell-type-specific gene expression in both endogenous and heterologous contexts, making them ideal for developing tools to access, study, and manipulate virtually any mammalian cell type.

Despite recent success in cataloging the gene expression profiles of distinct cell subpopulations in the nervous system, the limited ability to specifically access these subpopulations hinders the study of their function. For example, the mammalian cerebral cortex is composed of over one hundred cell types, most of which cannot be individually accessed using existing tools. Glutamatergic excitatory neuron cell types propagate electrical signals across neural circuits, whereas GABAergic inhibitory interneuron cell types play an essential role in cortical signal processing by modulating neuronal activity, balancing excitability, and gating information. Although relatively lower in abundance than excitatory neurons, interneurons are highly diverse; for example, somatostatin-expressing cortical interneurons comprise several anatomically, electrophysiologically, and molecularly defined cell types whose dysfunction is associated with neuropsychiatric and neurological disorders (see e.g., Jiang et al., 2015, Science 350:aac9462; Muñoz et al., 2017, Science 355:954-959; Tasic et al., 2018, Nature 563:72-78). Given the vast diversity of cell types in the brain, and the inability of current tools to access most neuronal cell types, enhancer-driven viral reagents are the next generation of cell-type-specific transgenic tools enabling facile, inexpensive, cross-species, and targeted observation and functional study of neuronal cell types and circuits.

Despite the potential of cell-type-specific enhancers to revolutionize neuroscience research, cell-type-restricted gene regulatory elements (GREs) have not yet been systematically identified. Moreover, functional evaluation of candidate GRE-driven viral vector expression across all cell types in the tissue of interest is currently laborious, expensive, and low-throughput, typically relying on the production of individual viral vectors and the assessment of expression across a limited number of cell types by in situ hybridization or immunofluorescence. The lack of a generalizable platform for rapid identification and functional testing of cell-type-specific enhancers is therefore a critical bottleneck impeding the generation of new viral reagents required to elucidate the function of each cell type in a complex organism.

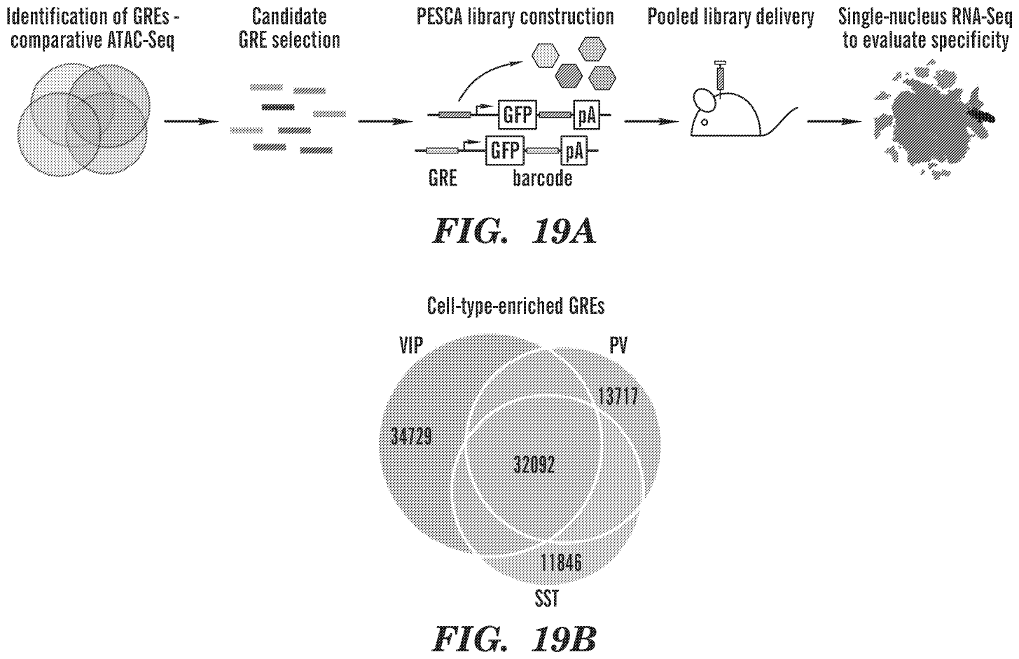

To address these issues, the principles of massively parallel reporter assays (MPRA) were merged with single-cell RNA sequencing (scRNA-seq) to develop a Paralleled Enhancer Single Cell Assay (PESCA) to identify and functionally assess the specificity of hundreds of GREs across the full complement of cell types present in the brain. In the PESCA protocol, the expression of a barcoded pool of AAV vectors harboring GREs is analyzed by single-nucleus RNA sequencing (snRNA-seq) to evaluate the specificity of each constituent GRE across tens of thousands of individual cells in the target tissue, through the use of an orthogonal cell-indexed system of transcript barcoding (see e.g., FIG. 1A, FIG. 19A).

The efficacy of PESCA was validated in the murine primary visual cortex by identifying GREs that confine AAV expression to somatostatin (SST)-expressing interneurons and showed that these vectors can be used to modulate neuronal activity selectively in SST neurons. SST neurons in the brain were chosen as the focus because this population is known to be diverse and to be composed of several relatively rare subpopulations (see e.g., Muñoz et al., 2017, supra; Tasic et al., 2018, supra; Tasic et al., 2016, supra), and thus serves as a good test case. As described below, these findings highlight the utility of PESCA for identifying viral constructs that drive gene expression selectively in a subset of neurons and establish PESCA as a platform of broad interest to the research and gene therapy community, permitting the generation of cell-type-specific AAVs for any cell type.

GRE Selection and Library Construction

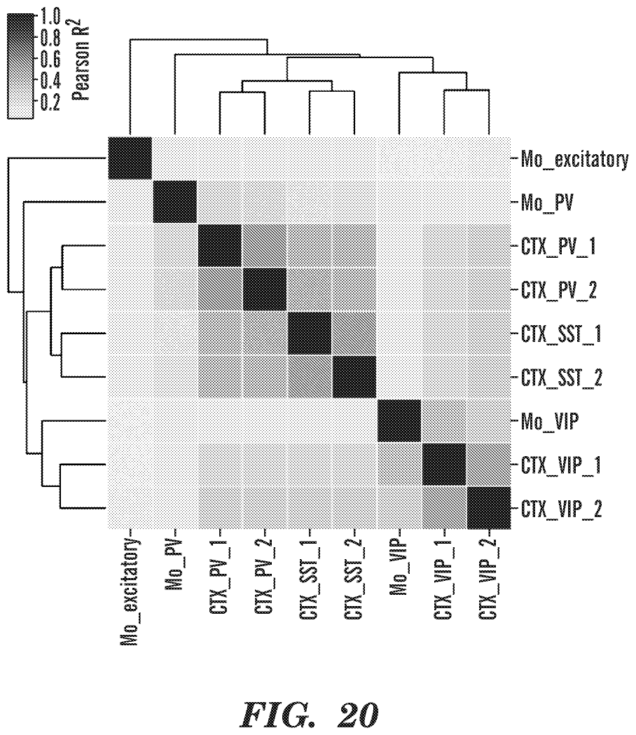

To identify candidate SST interneuron-restricted gene regulatory elements (GREs), comparative epigenetic profiling was conducted of the three largest classes of cortical interneurons: somatostatin (SST)-expressing, vasoactive intestinal polypeptide (VIP)-expressing and parvalbumin (PV)-expressing cells. To this end, the recently developed Isolation of Nuclei Tagged in specific Cell Types (INTACT) (see e.g., Mo et al., 2015 supra) method was employed to isolate purified chromatin from of each of these cell types from the cerebral cortex of adult (6-10 week-old) mice. The assay for transposase-accessible chromatin using sequencing (ATAC-Seq) (see e.g., Buenrostro et al., 2015, Nature 523:486-490), which identifies nucleosome-depleted gene regulatory regions, was then used to identify genomic regions with enhanced accessibility (i.e., peaks) in the SST (n=57,932), PV (n=61,108), and VIP (n=79,124) chromatin samples (see e.g., FIG. 1B, FIG. 1C, FIG. 19E, FIG. 20, Materials and methods). These datasets can be used as a resource to identify putative gene regulatory elements as candidates for driving cell-type-specific gene expression for the numerous subtypes of SST, PV or VIP-expressing interneurons across diverse cortical regions.

To enrich for GREs that could be useful reagents to study and manipulate interneurons across mammalian species, including humans, the analysis started with an expanded list of 323,369 genomic coordinates (see e.g., Supplementary file 1 of Hrvatin et al., A scalable platform for the development of cell-type-specific viral drivers, Elife. 2019 Sep. 23; 8. pii: e48089, the content of which is incorporated herein by reference in its entirety). The expanded list of 323,369 genomic coordinates represented a union of cortical neuron ATAC-seq-accessible regions identified across dozens of experiments (see e.g., Materials and methods). This initial set of 323,369 genomic coordinates was first filtered to exclude GREs with poor mammalian sequence conservation (see e.g., Materials and methods; Supplementary file 1 of Hrvatin et al, 2019, supra, FIG. 4). The remaining 36,215 genomic regions were ranked by an enrichment of ATAC-seq signal in the SST samples over PV/VIP (see e.g., Materials and methods), and the top 287 most enriched GREs were selected for functional screening to identify enhancers that drive gene expression selectively in SST interneurons of the primary visual cortex (see e.g., FIG. 1D, Table 3).

A PCR-based strategy was used to simultaneously amplify and barcode each GRE from mouse genomic DNA (see e.g., Materials and methods). To minimize sequencing bias due to the choice of barcode sequence, each GRE was paired with three unique barcode sequences. The resulting library of 861 GRE-barcode pairs was pooled and cloned into an AAV-based expression vector, with the GRE element inserted 5′ to a promoter driving a GFP expression cassette and the GRE-paired barcode sequences inserted into the 3′ untranslated region (UTR) of the GRE-driven transcript (see e.g., Materials and methods, FIG. 2A, FIG. 5). This configuration was chosen to maximize the retrieval of the barcode sequence during single-cell RNA sequencing, which primarily captures the 3′ end of transcripts. The human beta-globin promoter was chosen since it has previously been used in conjunction with an enhancer to drive strong and specific expression in cortical interneurons (see e.g., Dimidschstein et al., 2016, Nature Neuroscience 19:1743-1749), although the modular cloning strategy is compatible with the use of other promoters. The library was packaged into AAV9, which exhibits broad neural tropism and has previously been used to drive payload expression in cortical neurons (see e.g., Cearley and Wolfe, 2006, Molecular Therapy 13:528-537). The complexity of the resulting rAAV-GRE library was then confirmed by next generation sequencing, detecting 802 of the 861 barcodes (93.1%), corresponding to 285 of the 287 GREs (99.3%) (see e.g., FIG. 2B).

PESCA Screen Identifies GREs Highly Enriched for SST Interneurons

To quantify the expression of each rAAV-GRE vector across the full complement of cell types in the mouse visual cortex, a modified single-nucleus RNA-Seq (snRNA-Seq) protocol was used to first determine the cellular identity of each nucleus and then quantify the abundance of the GRE-paired barcodes in the transcriptome of nuclei assigned to each cell type. Two adjacent injections (800 nL each) of the pooled AAV library (1×10¹³ viral genomes/mL) were first administered to the primary visual cortex (V1) of two 6-week-old C57BL/6 mice. Twelve days following injection, the injected cortical regions were dissected and processed to generate a suspension of nuclei for snRNA-Seq using the inDrops™ platform (see e.g., Klein et al., 2015, supra; Zilionis et al., 2017, Nature Protocols 12:44-73; Materials and methods). A total of 32,335 nuclei were subsequently analyzed across the two animals, recovering an average of 866 unique non-viral transcripts per nucleus, representing 610 unique genes (see e.g., FIG. 6A, FIG. 22).

Since droplet-based high-throughput snRNA-Seq samples the nuclear transcriptome with low sensitivity (see e.g., Klein et al., 2015, supra), viral-derived transcripts were initially detected in only 3.9% of sampled nuclei. Therefore, a modified PCR-based approach was designed to enrich for barcode-containing viral transcripts, which yielded deep coverage of AAV-derived transcripts with simultaneous shallow coverage of the non-viral transcriptome. PCR enrichment increased the viral transcript recovery 382-fold in the sampled nuclei, to an average of 15.6 unique viral transcripts, 6.0 unique GRE-barcodes, and 5.7 unique GREs per cell (see e.g., FIG. 2C, FIG. 6C). Using this modified protocol, viral transcripts were identified across 86% of cells (see e.g., FIG. 6E), with a high correlation (r=0.9, p<2.2×10⁻¹⁶) observed between the abundance of each barcoded AAV in the library and the number of cells infected by that AAV (see e.g., FIG. 6F), suggesting that GRE sequences did not alter viral tropism and that GRE-driven vectors had broadly similar levels of expression. Only 0.3±0.06% (mean, stdev) of viral reads did not correspond to any of the known barcodes or could not be uniquely assigned to a barcode (within two mismatches), suggesting that this amplification strategy did not grossly change the composition of the viral library.

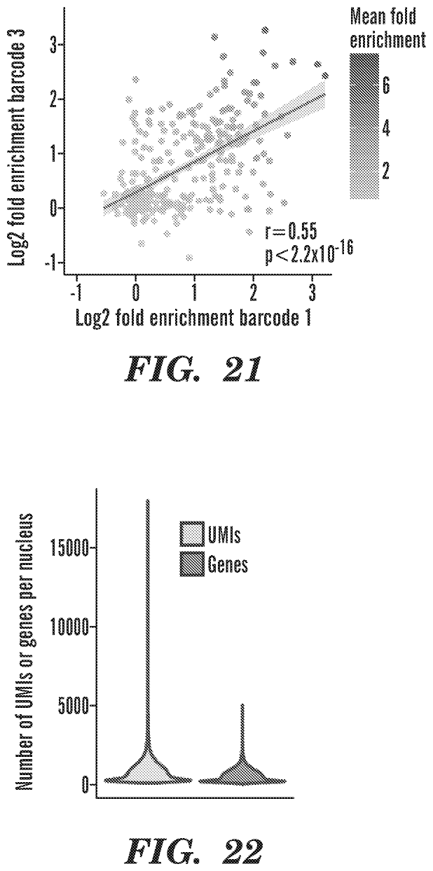

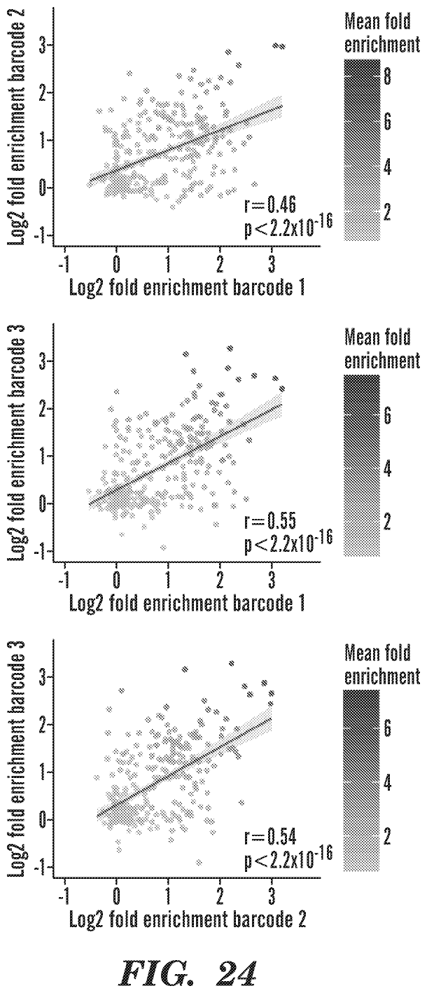

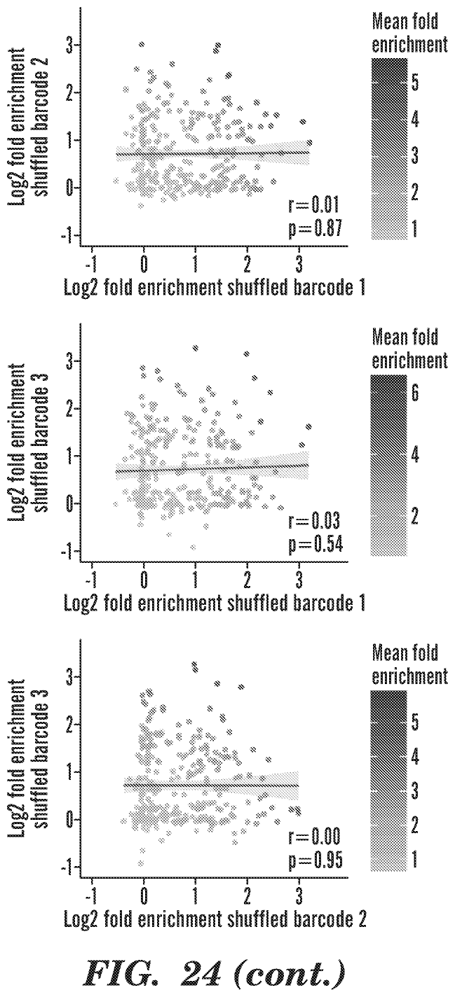

Nuclei were classified into ten cell types using graph-based clustering and expression of known marker genes (see e.g., Materials and methods; FIG. 2C, FIG. 2D, FIG. 7). The average expression of each viral-derived barcoded transcript was analyzed across all ten cell types, and an enrichment score was calculated from the ratio of expression in Sst nuclei compared to all Sst⁻ nuclei. As expected, sets of three barcodes associated with the same GRE showed highly statistically correlated enrichment scores (r=0.52±0.05, p<2.2×10⁻¹⁶) (see e.g., FIG. 2E, FIG. 21, FIG. 24), which were significantly lower when barcodes were randomly shuffled (shuffled r=0.002±0.06; Wilcox test between data and shuffled data, p=0.003).

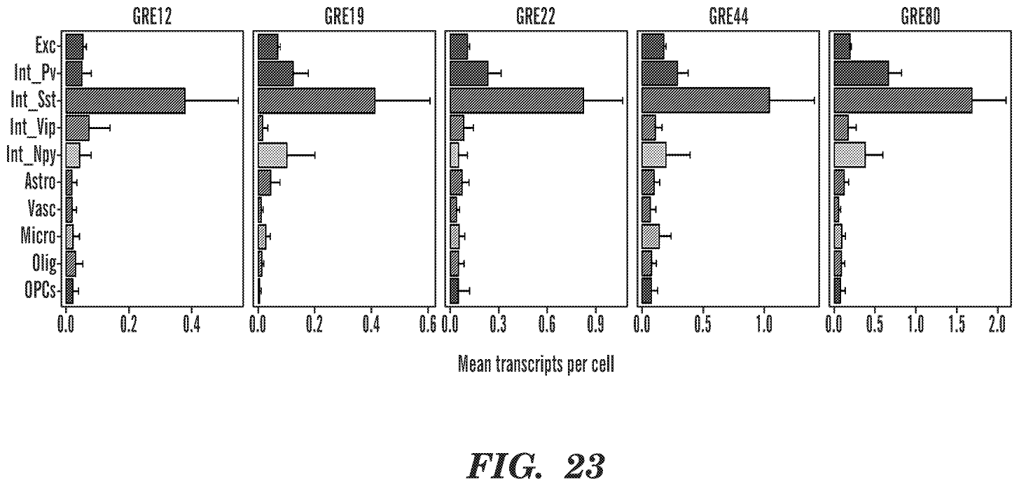

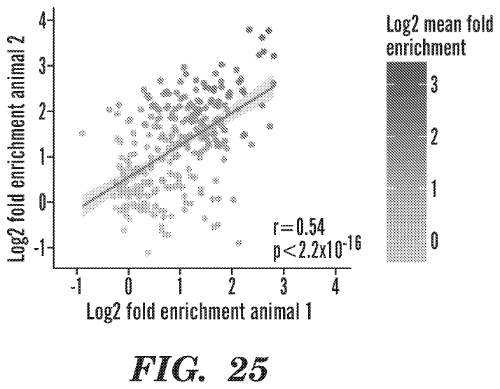

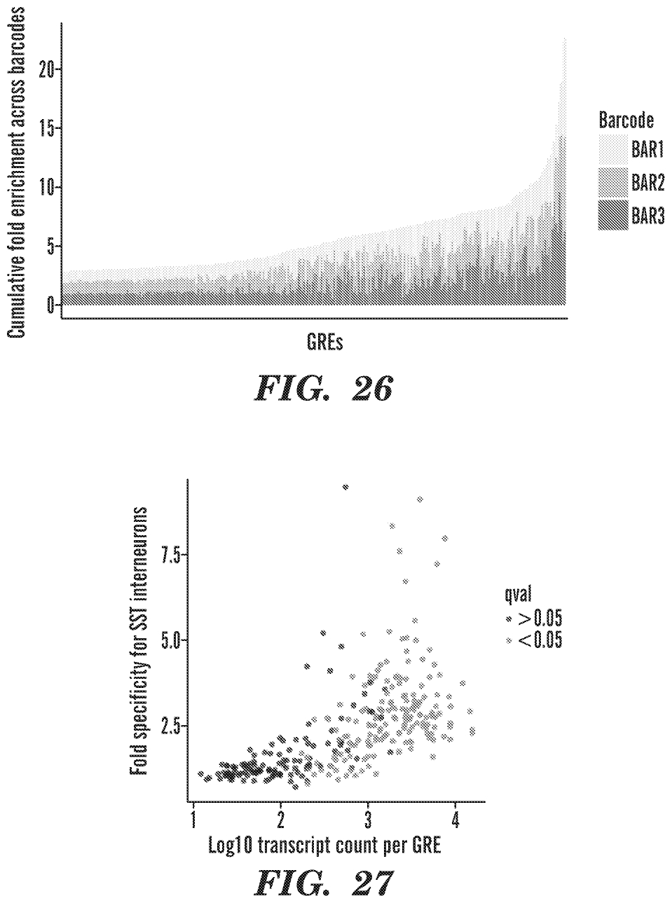

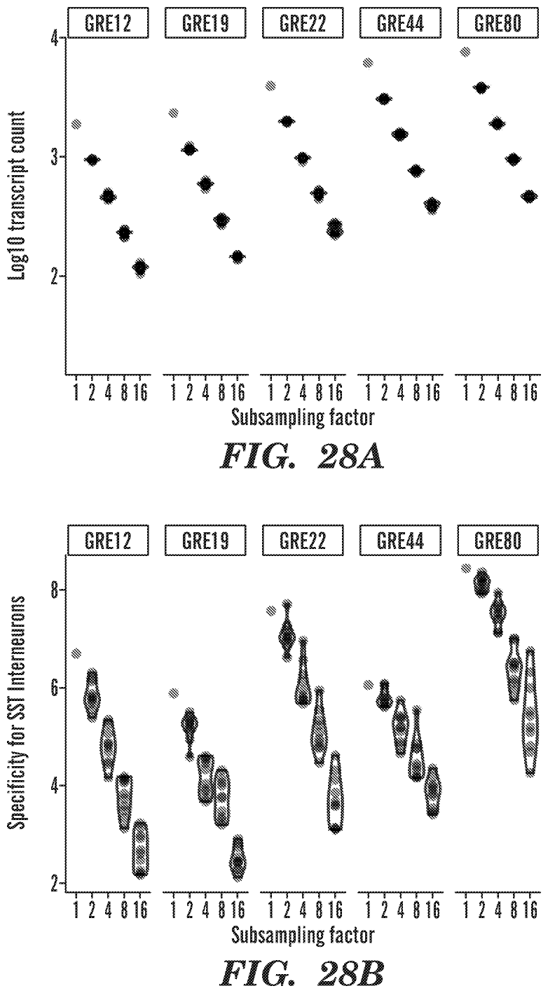

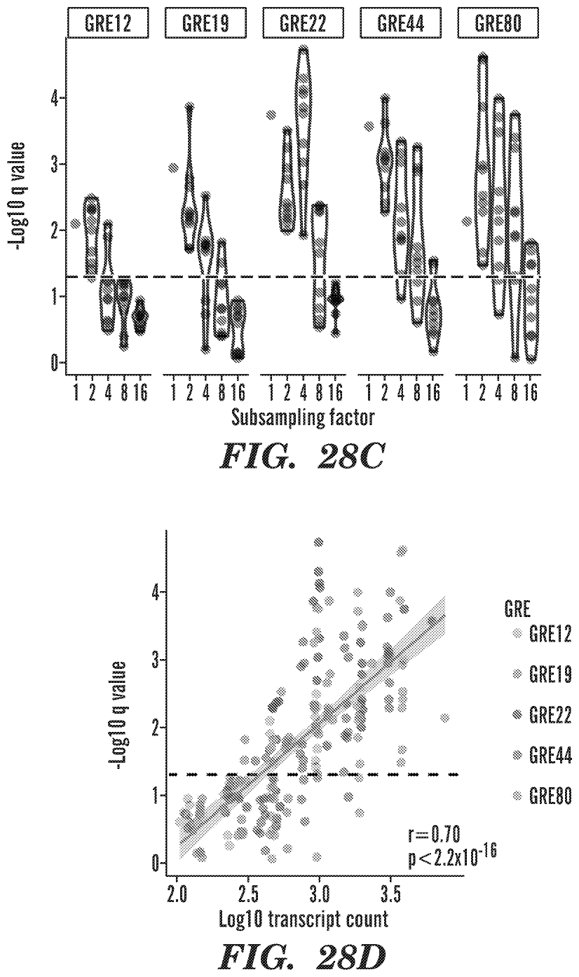

Having confirmed a robust, non-random correlation in enrichment scores among the three barcodes associated with each GRE, a single expression value was next computed for each of the 287 viral drivers by aggregating expression data from three barcodes associated with the same GRE, and differential gene expression analysis was conducted between Sst and Sst⁻ cells for each rAAV-GRE. Differential gene expression analysis between Sst⁺ and Sst⁻ cells for each rAAV-GRE revealed a marked overall enrichment of viral-derived transcripts in the Sst subpopulation (see e.g., FIG. 9A). As expected, a high correlation was observed between GRE-specific enrichment scores across two animals (r=0.54, p<2.2×10⁻¹⁶) (see e.g., FIG. 25). Among the 287 GREs tested, several viral drivers were identified that promoted highly specific reporter expression in the Sst subpopulation (q<0.01, fold-change>7; see e.g., FIG. 2H, FIG. 2I, FIG. 2J, FIG. 9B, FIG. 23, FIG. 26). To assess how the abundance of each GRE in the library impacts the ability to detect cell-type-specific expression, the specificity of each GRE was analyzed as a function of the number of transcripts retrieved. Highly abundant GRE-driven transcripts were more likely to be significantly enriched in SST⁺ cells, suggesting that there may not have been sufficient power to assess the cell-type-specificity of the less abundant GREs in the library (see e.g., FIG. 27). Consistent with this observation, computationally subsampling the number of viral transcripts across the most cell-type-specific GREs gradually reduced the ability to statistically detect their enrichment in Sst cells (see e.g., FIG. 28A-28D). These observations indicate that the expression of sparsely detected GRE-driven transcripts may not be sufficient to allow evaluation of cell-type-specificity and that increasing sequencing depth can permit the screening and evaluation of a larger number of GREs.

In Situ Characterization of rAAV-GRE Reporter Expression

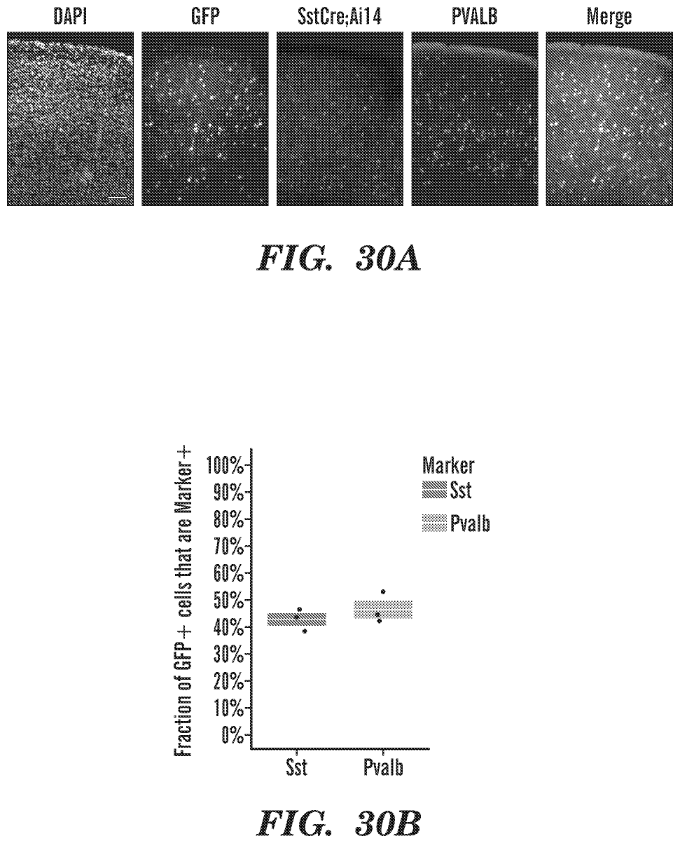

In order to validate the cell-type-specificity of the resulting hits using methods that do not rely on single-cell sequencing-based approaches, three of the top five viral drivers (GRE12, GRE22, GRE44), as well as a control viral construct lacking the GRE element (AGRE), were selected for injection into V1 of adult transgenic Sst-Cre; Ai14 mice, in which SST⁺ cells express the red fluorescent marker tdTomato (see e.g., SEQ ID NOs: 10-12). Fluorescence analysis twelve days following injection with rAAV-[GRE12, GRE22 or GRE44]-GFP revealed strong yet sparse GFP labeling centered around cortical layers IV and V (see e.g., FIG. 3A-3C). By contrast, the control rAAV-AGRE-GFP showed a strikingly different pattern of GFP expression concentrated around the sites of injection, with expression in a larger number of cells (see e.g., FIG. 3D). Many rAAV-GRE12/22/44-GFP virally infected cells were SST-positive, as indicated by the high degree of overlapping GFP and tdTomato expression: 90.7±2.1% for rAAV-GRE12-GFP (170 cells, four animals); 72.9±4.2% for rAAV-GRE22-GFP (1164 cells, three animals), and 95.8±0.6% for rAAV-GRE44-GFP (759 cells, four animals). (see e.g., FIG. 3E-3F, FIG. 10). By contrast, 27.2±1.9% of GFP⁺ cells also expressed tdTomato following rAAV-AGRE-GFP infection (2066 cells, three animals; see e.g., FIG. 3E-3F). Although the 27.2% overlap between rAAV-AGRE-GFP expression and SST⁺ cells suggests that the vector has some baseline preference for SST⁺ interneurons, the insertion of GRE12, GRE22 and GRE44 serves to effectively restrict AAV payload expression to SST⁺ interneurons. To show that the viral backbone could drive expression in non-SST cell types with the appropriate enhancer, the mDlx5/6 enhancer whose expression was restricted to a broader population of inhibitory neurons (see e.g., Dimidschstein et al., 2016, supra) was cloned into the viral backbone. The rAAV2/9-mDlx5/6-GFP vector was injected into Sst-Cre; Ai14 mice, and 57.1% of GFP⁺ cells were not positive for tdTomato (1977 cells, three animals; see e.g., FIG. 30A-30B).

It is notable that the GREs not only promote expression in SST⁺ cells but also greatly reduce background expression in SST cells, indicating both enhancer and repressor functionality. Without wishing to be bound by theory, consistent with this hypothesis, the incorporation of GRE12, GRE22 and GRE44 into the rAAV both increased the number of SST⁺ GFP⁺ cells (1.7-2-fold) and dramatically (3-32-fold) decreased the number of SST⁻ cells that expressed GFP (see e.g., FIG. 3G, FIG. 11). To further investigate the specificity of the viral drivers among cortical interneuron cell types each construct was injected into Vip-Cre; Ai14⁺ mice in which all VIP⁺ cells express tdTomato, and used fluorescence antibody staining to label PV-expressing cells (see e.g., FIG. 12). Fluorescent signal analysis indicated the percentage of GFP⁺ cells that were either VIP⁺ or PV⁺ (rAAV-SST12-GFP⁺ [2.6±2.6%], rAAV-GRE22-GFP⁺ [3.5±2.0%] and rAAV-GRE44-GFP⁺ [6.0±2.7%]; see e.g., FIG. 3H). These findings confirm that among major interneuron cell classes, all three GRE-driven vectors are highly SST-specific.

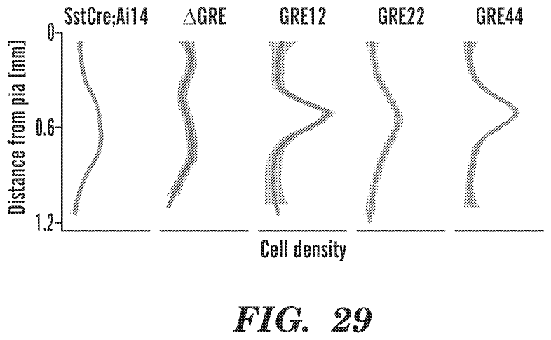

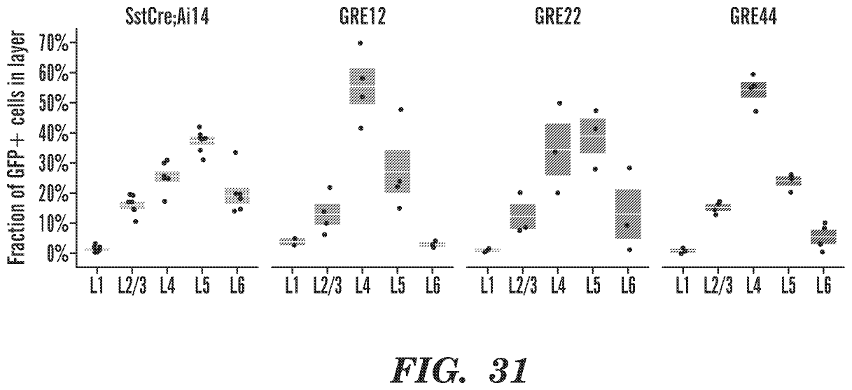

Because at least five subtypes of cortical SST⁺ interneurons have previously been identified based on the laminar distribution of their cell bodies and projections (see e.g., Muñoz et al., 2017, supra; Urban-Ciecko and Barth, 2016, Nature Reviews Neuroscience 17:401-409), the laminar distribution of GFP-expressing cells was investigated for the three SST-enriched viral drivers. Intriguingly, the majority of rAAV-GRE12-GFP⁺ and rAAV-GRE44-GFP⁺ SST⁺ cells were found to reside in layers IV and V, which was distinct from the distribution observed for the full SST⁺ cell population in visual cortex (p=1.3×10⁻⁶, p<2.2×10⁻¹⁶, respectively, Mann-Whitney U test, two-tailed; see e.g., FIG. 3I, FIG. 29, FIG. 31). By contrast, rAAV-AGRE-GFP was expressed in SST⁺ cells as well as other neuronal subtypes across all layers, indicating that increased labeling of rAAV-GRE12-GFP and rAAV-GRE44-GFP in layer IV and V was due to restricted gene expression and not restricted viral tropism.

Electrophysiological Characterization of rAAV-GRE-GFP-Expressing SST Subtypes

In addition to variability in laminar distribution, different electrophysiological phenotypes have also been observed in cortical SST interneurons (see e.g., Ma et al., 2006, Journal of Neuroscience 26:5069-5082; Tremblay et al., 2016, Neuron 91:260-292). To determine whether AAV-GRE reporters can be used to distinguish electrophysiologically distinct SST subtypes, the most cell-type-restricted construct, rAAV-GRE44-GFP, was injected into the visual cortex of adult Sst-Cre; Ai14 mice and whole-cell current-clamp recordings were obtained from double GFP- and tdTomato-positive neurons (rAAV-GRE44-GFP⁺), as well as immediately nearby tdTomato-positive but GFP-negative cells (rAAV-GRE44-GFP⁻).

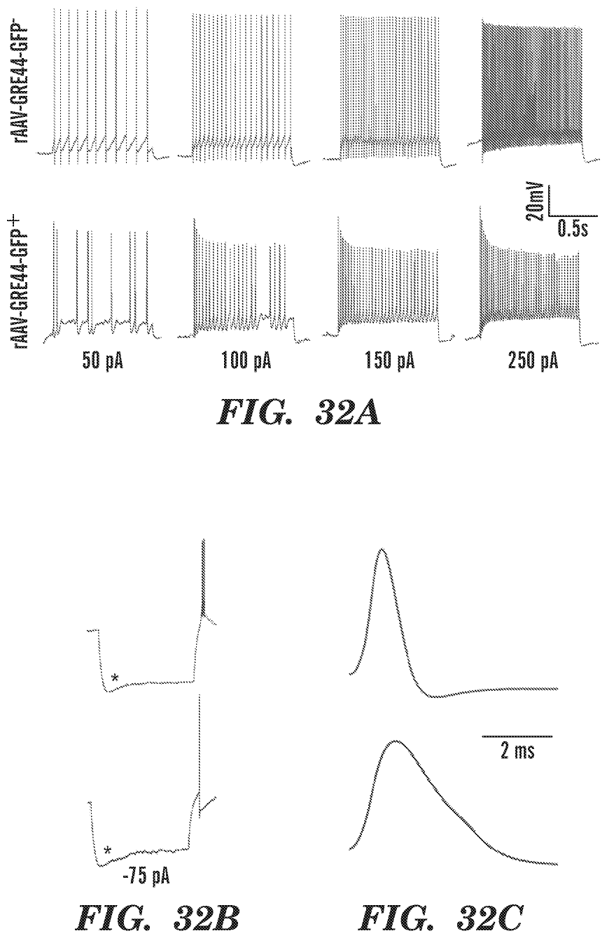

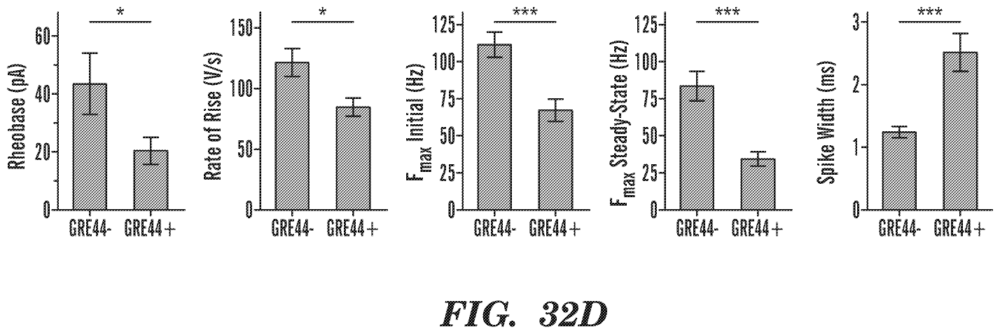

The recordings indicate that both rAAV-GRE44-GFP⁺ and rAAV-GRE44-GFP⁻ SST⁺ neurons display the properties of adapting SST interneurons with high input resistances and features consistent with those previously reported for deep layer cortical SST neurons (see e.g., Ma et al., 2006, supra; Xu et al., 2013, Neuron 77:155-167; see e.g., FIG. 32A-32B). However, rAAV-GRE44-GFP⁺ SST neurons were distinct with respect to several electrophysiological parameters. The action potentials of rAAV-GRE44-GFP⁺ SST neurons were significantly broader than those of rAAV-GRE44-GFP⁻ SST neurons (see e.g., FIG. 32C-32D), perhaps due to differences in expression of specific channels in these subgroups of SST neurons, such as voltage-activated potassium channels, and BK calcium-activated potassium channels (see e.g., Bean, 2007, Nature Reviews Neuroscience 8:451-465; Kimm et al., 2015, Journal of Neuroscience 35:16404-16417). Furthermore, rAAV-GRE44-GFP⁺ SST neurons had a lower rheobase and fired action potentials with a slower rising phase, and at lower maximal frequencies compared to rAAV-GRE44-GFP⁻ SST neurons (see e.g., FIG. 32A, FIG. 32D, Table 4). Although it cannot be confirmed that GRE44 expression is restricted to a specific transcriptionally defined subtype of SST interneurons, these electrophysiology experiments further emphasize the ability of PESCA to target functionally distinct subgroups of previously defined interneuron types.

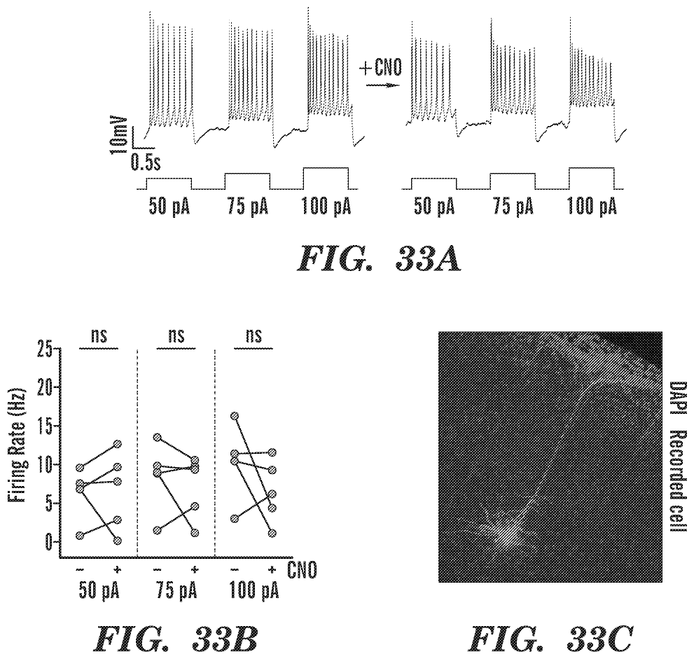

Finally, it was evaluated whether the identified SST⁺ neuron-restricted viral drivers support sufficiently high and persistent levels of payload expression to effectively modulate SST⁺ cell physiology. Designer receptors exclusively activated by designer drugs (DREADDs) are a commonly employed viral payload used to dynamically regulate neuronal activity in response to the synthetic ligand clozapine-N-oxide (CNO) (see e.g., Armbruster et al., 2007, PNAS 104:5163-5168). Therefore, the visual cortex of adult wild-type mice (6-8 week-old) was injected with rAAV-GRE12-Gq-DREADD-tdTomato, a construct in which GRE12 drives the expression of an activating DREADD as well as tdTomato (see e.g., SEQ ID NO: 22). GRE12 was chosen for this assay as it drives the weakest expression of the three evaluated GREs (see e.g., FIG. 2E, FIG. 2J) and thus, if it effectively drives DREADD expression, the other GREs would be expected to as well. Electrophysiological recordings were obtained from tdTomato⁺ cells of acute cortical slices in a whole-cell, current-clamp configuration two weeks post-injection. All tdTomato cells showed striking sensitivity to CNO, as indicated by significantly increased firing rates in response to depolarizing current steps and depolarized resting membrane potentials (see e.g., FIG. 3K-3M). To ensure that increases in firing rate upon CNO application were specific to infected SST⁺ neurons, recordings were obtained from nearby uninfected pyramidal neurons that were identified by morphology, and it was found that there was no statistically significant increase in firing rate upon CNO application (see e.g., FIG. 33A-33C). These data demonstrate the ability of GRE-driven SST⁺ neuron-specific reagents to robustly and specifically modulate the activity of SST⁺ cells in non-transgenic animals.

TABLE 4
Electrophysiological Parameters of GRE44− and GRE44+ SST Neurons
in Visual Cortex (values are shown as mean ± SEM).

| | | | p value |
|---|---|---|---|
| | | | (2-tailed |
| | GRE44− | GRE44+ | unpaired |
| | (n = 16) | (n = 16) | t-test) |
| Vᵣₑₛₜ (mV) | −62.4 ± 1.51 | −60.6 ± 1.63 | 0.41 |
| Rᵢₙ (MΩ) | 304 ± 54.8 | 391 ± 47.3 | 0.24 |
| τₘ (ms) | 14.2 ± 2.35 | 22.8 ± 4.32 | 0.094 |
| Threshold (mV) | −45.6 ± 1.24 | −48.1 ± 1.26 | 0.17 |
| AP Peak (mV) | 13 ± 3.38 | 11.6 ± 3.19 | 0.76 |
| AP Trough (mV) | −63.6 ± 1.36 | −63.7 ± 1.43 | 0.96 |
| AP Height (mV) | 76.5 ± 4.46 | 75.2 ± 4.06 | 0.83 |
| Rate of Rise (V/s) | 122 ± 11.7 | 85 ± 7.34 | 0.013* |
| Rheobase (pA) | 43.5 ± 10.4 | 20.3 ± 4.64 | 0.044* |
| Spike Half-Width (ms) | 1.25 ± 0.0819 | 2.52 ± 0.307 | 0.0004*** |
| Fₘₐₓ, steady-state (Hz) | 83.4 ± 9.8 | 34.5 ± 4.32 | 0.0002*** |
| Fₘₐₓ, initial (Hz) | 111 ± 8.35 | 67.3 ± 7.48 | 0.0007*** |
| Spike adaptation ratio | 0.763 ± 0.0839 | 0.561 ± 0.0699 | 0.08 |

### DISCUSSION

The PESCA platform extends previous paralleled reporter assays carried out using bulk tissue or sorted cells by including a single-cell RNA-seq-based readout to evaluate the cell-type-specificity of gene expression. This represents a significant advancement over current approaches to viral vector design, as it permits the rapid in vivo screening of hundreds of GREs for enhanced cell-type-specificity without needing transgenic tools to evaluate their specificity. In this study, PESCA was applied to identify enhancer elements that robustly and specifically drive gene expression in a rare SST⁺ population of GABAergic interneurons in the mouse central nervous system. Since the vectors used in this PESCA screen in the absence of GREs show broad expression in the murine V1, the identified GREs function to both enhance and restrict viral expression.

The selection of candidate GREs for screening can benefit from the systematic profiling of additional cell types by traditional or single-cell ATAC-Seq methods. In this regard, consideration of a published ATAC-Seq dataset from excitatory neurons (see e.g., Mo et al., 2015, supra) can be used to refine the starting GRE set by excluding approximately half of the screened GREs from the initial pool. This is particularly relevant insofar as the ability to assess the GRE library depends on the number of cells sequenced from the target and non-target populations and the sequencing depth, as the coverage of each GRE is inversely proportional to the number of GREs screened. In the screen described here, there is sufficient power to assess approximately ⅔ of the 287 GREs at the reported sequencing depth (see e.g., FIG. 2J, FIG. 9A-9B, FIG. 25-27).

Using a robust method of specifically isolating RNA from the target cell population, screening the PESCA library by sequencing pooled RNA from all target versus all non-target cells provides a less expensive and more scalable approach. However, by averaging across multiple non-target cell types, such an approach could be confounded by the presence of rare, highly expressing non-target cells.

Finally, once candidate PESCA hits have been identified, several follow-up assays at multiple titers can be used to identify which among these hits have the desired intensity and specificity of protein expression. In this regard, the snRNA-seq PESCA screen identified GRE12, GRE22 and GRE44 as 8.3-, 9.1- and 7.2-fold more highly expressed in SST⁺ compared to SST⁻ cells, respectively, whereas these GREs showed distinct specificity for SST⁺ cells (91%, 73% and 96% respectively; see e.g., FIG. 3F) when evaluated at the protein level.

Given current evidence that the mechanisms of gene regulatory element function are conserved across tissues and species, PESCA can be readily applied to other neuronal or non-neuronal cell types, diverse model organisms, tissues, and viral types. Moreover, single-cell screening approaches are not limited to GRE screening; PESCA can be easily adapted to assess the cell-type-specificity of viral capsid variants or other mutable aspects of viral design. Indeed, the PESCA library cloning strategy is largely vector- and capsid-independent, allowing for the use of different promoters or serotypes. The choice of capsid and promoter was driven by previous work using AAV9 and the minimal beta-globin promoter to drive expression in cortical interneurons (see e.g., Dimidschstein et al., 2016, supra). Different capsids or promoter can be used for targeting this and other cell types.

In conclusion, this study addresses the urgent practical need for new tools to access, study, and manipulate specific cell types across complex tissues, organ systems, and animal models by providing a screening platform that can be used to rapidly supply such tools as needed. Moreover, as the promise of gene therapy to treat and cure a broad range of diseases is being realized, PESCA can pave the way for a new generation of targeted gene therapy vehicles for diseases with cell-type-specific etiologies, such as congenital blindness, deafness, cystic fibrosis, and spinal muscular atrophy.

Materials and Methods

TABLE 2
Key resources

| Reagent type | | Source or | | Additional |
|---|---|---|---|---|
| (species) or resource | Designation | reference | Identifiers | information |
| Gene (Mus musculus) | Sst | | NCBI ™ Gene ID: 20604 | |
| Genetic reagent | Sst-IRES-Cre | Jackson | IMSR (International Mouse Strain | |
| (M. musculus) | | Laboratory ™ | Resource) (Cat# JAX: 013044, RRID | |
| | | Stock # 013044 | (Research Resource Identifiers): | |
| | | | IMSR_JAX: 013044 | |
| Genetic reagent | Vip-IRES-Cre | The Jackson | IMSR Cat# JAX: 010908, | |
| (M. musculus) | | Laboratory ™ | RRID: IMSR_JAX: 010908 | |
| | | Stock # 010908 | | |
| Genetic reagent | Pv-Cre | The Jackson | IMSR Cat# JAX: 017320, | |
| (M. musculus) | | Laboratory ™ | RRID: IMSR_JAX: 017320 | |
| | | Stock # 017320 | | |
| Genetic reagent | SUN1-2xsfGFP- | The Jackson | IMSR Cat# JAX: 021039, | |
| (M. musculus) | 6xMYC | Laboratory ™ | RRID: IMSR_JAX: 021039 | |
| | | Stock # 021039 | | |
| Genetic reagent | Ai14 | The Jackson | IMSR Cat# JAX: 007914, | |
| (M. musculus) | | Laboratory ™ | RRID: IMSR_JAX: 007914 | |
| | | Stock # 007914 | | |
| Strain, strain | High Efficiency | New England | C2987H | Competent |
| background | NEB 5-alpha ™ | Biolabs ™ | | cells |
| (Escherichia coli) | | | | |
| Antibody | anti-GFP (Rabbit | Thermo Fisher ™ | Cat# G10362; | 0.012 ug/ul |
| | monoclonal) | | RRID: AB_2536526 | |
| Antibody | anti-Parvalbumin | EMD Millipore ™ | Cat# MAB1572; | IF(1:2000) |
| | (Mouse | | RRID: AB_2174013 | |
| | monoclonal) | | | |
| Recombinant | pAAV-mDlx- | See e.g., | Addgene ™ # 83900; | |
| DNA reagent | GFP-Fishell-1 | Dimidschstein et | RRID: Addgene_83900 | |
| | (plasmid) | al., 2016, supra | | |
| Recombinant | pAAV-ΔGRE - | Herein, see e.g., | | |
| DNA reagent | GFP- (plasmid) | SEQ ID NO: 10 | | |
| Recombinant | pAAV-GRE12- | Herein, see e.g., | | |
| DNA reagent | GFP- (plasmid) | SEQ ID NO: 11 | | |
| Recombinant | pAAV-GRE22- | Herein, see e.g., | | |
| DNA reagent | GFP- (plasmid) | SEQ ID NO: 12 | | |
| Recombinant | pAAV-GRE44- | Herein, see e.g., | | |
| DNA reagent | GFP- (plasmid) | SEQ ID NO: 13 | | |
| Commercial | Nextera DNA | Illumina ™ | FC-121-1030 | |
| assay or kit | Library Prep | | | |
| | Kit ™ | | | |
| Commercial | In-Fusion HD | Takara Bio ™ | 639645 | |
| assay or kit | cloning kit ™ | | | |
| Commercial | Agencourt | Beckman | # A63881 | |
| assay or kit | AMPure XP ™ | Coulter ™ | | |
| Commercial | Hot Start High- | New England | M0494L | |
| assay or kit | Fidelity Q5 | Biolabs ™ | | |
| | polymerase ™ | | | |

Mice: Animal experiments were approved and followed ethical guidelines. For INTACT, the following: Sst-IRES-Cre (The Jackson Laboratory™ Stock #013044), Vip-IRES-Cre (The Jackson Laboratory Stock #010908) and Pv-Cre (The Jackson Laboratory™ Stock #017320) were crossed with SUN1-2xsfGFP-6xMYC (The Jackson Laboratory Stock #021039), and adult (6-12 wk old) male and female F1 progeny were used. For PESCA screening adult (6-10 wk) C57BL/6J (The Jackson Laboratory™, Stock #000664) mice were used. For confirmation of hits Sst-IRES-Cre (The Jackson Laboratory™ Stock #013044) or Vip-IRES-Cre (The Jackson Laboratory™ Stock #031628) mice were crossed with Ai14 mice (The Jackson Laboratory™ Stock #007914), and adult (6-12 wk old) male and female F1 progeny were used. All mice were housed under a standard 12 hr light/dark cycle.

INTACT purification and in vitro transposition: INTACT employs a transgenic mouse that expresses a cell-type-specific Cre and a Cre-dependent SUN1-2xsfGFP-6xMYC (SUN1-GFP) fusion protein. Nuclear purifications were performed from whole cortex of adult mice as previously described using anti-GFP antibodies (Fisher G10362) (see e.g., Mo et al., 2015, supra; Stroud et al., 2017, supra). Isolated nuclei were gently resuspended in cold L1 buffer (50 mM Hepes pH 7.5, 140 mM NaCl, 1 mM EDTA, 1 mM EGTA, 0.25% Triton™ X-100, 0.5% NP40, 10% Glycerol, protease inhibitors), and pelleted at 800 g for 5 min at 4° C. DNA libraries were prepared from the nuclei using the Nextera DNA Library Prep Kit™ (Illumina™) according to manufacturer's protocols. The final libraries were purified using the Qiagen MinElute™ kit (Cat #28004) and sequenced on a Nextseq 500™ benchtop DNA sequencer (Illumina™). For each of the three inhibitory subtypes examined, two independent ATAC-seq experiments were performed, each on Sun-positive nuclei isolated from a single animal. The nuclei were not counted prior to performing ATAC-seq, as yields were low enough that the process of counting would remove a large fraction of isolated nuclei and negatively impact the quality of the ATAC-seq experiment. However, during the process of establishing the Su1 IP protocol, 20-30 k nuclei were consistently counted per animal.

ATAC-seq mapping: All ATAC-seq libraries were sequenced on the Nextseq 500™ benchtop DNA sequencer (Illumina™). Seventy-five base pair (bp) single-end reads were obtained for all datasets. ATAC-seq experiments were sequenced to a minimum depth of 20 million (M) reads. Reads for all samples were aligned to the mouse genome (e.g., GRCm38/mm10, December 2011) using default parameters for the Subread (subread-1.4.6-p3) (see e.g., Liao et al., 2013, supra) alignment tool after quality trimming with Trimmomatic v0.33 (see e.g., Bolger et al., 2014, supra) with the following command: java -jar trimmomatic-0.33.jar SE -threads 1-phred33 [FASTQ_FILE] ILLUMINACLIP:[ADAPTER_FILE]:2:30:10 LEADING:5 TRAILING:5 SLIDINGWINDOW: 4: 20 MINLEN: 45. Nextera™ adapters were trimmed out for ATAC-seq data. Duplicates were removed with samtools rmdup. To generate UCSC genome browser tracks for ATAC-seq visualization, BEDtools was used to convert output bam files to BED format with the bedtools bamtobed command. Published mm10 blacklisted regions (see e.g., Schneider et al., 2017, supra) were filtered out using the following command: bedops -not-element-of 1 [BLACKLIST_BED]. Filtered BED files were scaled to 20 M reads and converted to coverageBED format using the BEDtools genomecov command: bedGraphToBigWig (UCSC-tools) was used to generate bigWIG files for the UCSC genome browser.

ATAC-seq peak calling and quantification: Two independent peak calling algorithms were employed to ensure robust, reproducible peak calls. First, tag directories were created using HOMER makeTagDirectory for each replicate, and peaks were called using default parameters for findPeaks with —style factor. MACS2 was also called using default parameters on each replicate. The summit files output by MACS2 were converted to bed format and each summit extended bidirectionally to achieve a total length of 300 bp. As the ATAC-seq peak calls would ultimately be used to identify a small subset of highly enriched regulatory elements for subsequent screening, it was required that a peak be called independently by both approaches in a given replicate for its inclusion in the final peak list for that sample. This approach reduced the rate of false positive peak calls.

Beyond the ATAC-seq data described herein (in SST, VIP, and PV populations several additional ATAC-seq experiments have been carried out across cortical regions and cell types (e.g., DRD3, GPR26, NTSR1, SCNN1, CDH5, RBP4, RORB Cre driver×Sun1 crosses; data not shown). To produce a final list of reference coordinates containing 323,369 genomic regions that were accessible in at least one sample, the MACS2/HOMER-intersected peak bed files for each experimental replicate were unioned using the bedops --everything command. Bedtools merge was then used to combine any peaks that overlapped in this unioned bed file; in this way, any region that was significantly called a peak in at least one ATAC-seq dataset was incorporated in the final aggregated peak list of 323,369 neuronal ATAC-seq peaks. The featurecounts package was then used to obtain ATAC-seq read counts for each of these accessible putative GREs, for downstream enrichment analyses.

Identification of conserved GREs: To identify GREs whose sequence is highly conserved across mammals, an appropriate conservation score was first identified to use as a threshold for high conservation. By analyzing the conservation of DNA sequences of the same length, but an arbitrary distance of 100,000 bases away from each identified GRE, a set of DNA sequences was generated whose conservation could be used to determine this threshold.

To this end, conservation scores for the 323,369 putative GREs and corresponding GRE-distal sequences were calculated using the bigWigAverageOverBed command to determine the average PhyloP score of each sequence based on mm10.60way.phyloP60wayPlacental.bw PhyloP scores (see e.g., available on the world wide web hgdownload.cse.ucsc.edu/goldenpath/mm10/phyloP60way/; see e.g., Pollard et al., 2010, Genome Research 20:110-121). After plotting the conservation score (phyloP, 60 placental mammals) of 323,369 GRE-distal sequences, the conservation score of the 95^{th} percentile of this distribution (PhyloP score=0.5) was determined and chosen as a minimal conservation score needed to classify any GRE as conserved. Using this cutoff, 36,215 GREs were classified as conserved and used for subsequent identification of SST-enriched GREs.

Identification of SST-enriched GREs: The genomic coordinates of 36,215 conserved GREs were used to quantify the ATAC-Seq signal from SST+, VIP+ and PV+ cells. A matrix was constructed representing the mean ATAC-Seq signal in SST+, VIP+ and PV+ cells for each of the 36,215 GREs and normalized such that the total ATAC-Seq signal from each cell population was scaled to 10⁷. Fold-enrichment was calculated for each region/GRE as [(Signal in cell type A)+0.5]/[mean(signal in cell types B and C)+0.5]. GREs were subsequently ranked based on fold-enrichment score.

Viral barcode design: Viral barcode sequences were chosen to be at least three insertions, deletions, or substitutions apart from each other to minimize the effects of sequencing errors on the correct identification of each barcode. The R library ‘DNAbarcodes’ and following functions were used: initialPool=create.dnabarcodes(10, dist=3, heuristic=‘ashlock’); finalPool=create.dnabarcodes(10, pool=initialPool, metric=‘seqlev’);

The result was a list of 1164 10-base barcodes that fit the initial criteria.

Amplification of GREs and barcoding is described below.

Genomic PCR: PCR primers were designed using primer3 2.3.7 such that a 150-400 bp flanking sequence was added to each side of the GRE. The forward primers contained a 5′ overhang sequence for downstream in-Fusion™ (Clonetech™) cloning into the AAV vector (SEQ ID NO: 1-5′-GCCGCACGCGTTTAAT). The reverse primers contained a 5′ overhang sequence containing the recognition sites for AsiSI and SalI restriction enzymes (SEQ ID NO: 2-5′-GCGATCGCTTGTCGAC). Hot Start High-Fidelity Q5™ polymerase (NEB™) was used according to manufacturer's protocol with mouse genomic DNA as template.

Barcoding PCR: The unpurified PCR products from the genomic PCR were used as templates for the barcoding PCR. A forward primer containing the sequence for downstream in-Fusion™ (Clonetech™) cloning into the AAV vector (SEQ ID NO: 3-5′-CTGCGGCCGCACGCGTTTA) was used in all reactions. Reverse primers were constructed featuring (in the 5′ →3′direction): 1) a sequence for downstream in-Fusion™ (Clonetech™) cloning into the AAV vector (SEQ ID NO: 4-5′-GCCGCTATCACAGATCTCTCGA), 2) a unique 10-base barcode sequence, and 3) sequence complementary with the AsiSI and SalI restriction enzyme recognition sites that were introduced during the first PCR (SEQ ID NO: 5-5′-GCGATCGCTTGTCGAC). Three different reverse primers were used for each of the GREs amplified during the genomic PCR. Hot Start High-Fidelity Q5™ polymerase (NEB™) was used according to the manufacturer's protocol.

PESCA library cloning: All PCR reactions were pooled and the amplicons purified using Agencourt AMPure XP™. The pAAV-mDlx-GFP-Fishell-1 is available from Addgene™ (plasmid #83900). The plasmid was digested with PacI and XhoI, leaving the ITRs and the polyA sequence. in-Fusion™ was used to shuttle the pool of GRE PCR products into the vector. Following transformation into High Efficiency NEB™ 5-alpha Competent E. coli and recovery, SalI and AsiSI were used to linearize the AAV vector containing the GREs. The expression cassette containing the human HBB promoter and intron followed by GFP and WPRE was isolated by PCR amplification from pAAV-mDlx-GFP-Fishell-1. The expression cassette was ligated with the linearized GRE-library-containing vector using T4 ligase and transformed into High Efficiency NEB 5-alpha Competent E. coli to yield the final library. 50 colonies were Sanger sequenced to determine the correct pairing between GRE and barcode and the correct arrangement of the AAV vector.

AAV preparation: The pooled PESCA library or individual AAV constructs (100 μg) were packed into AAV9. The titers (2-50×10¹³ genome copies/mL) were determined by qPCR. Next generation sequencing using the NextSeq 500 platform was used to determine the complexity of the pooled PESCA library (se e.g., FIG. 2A).

VI cortex injections: Animals were anesthetized with isoflurane (1-3% in air) and placed on a stereotactic instrument (Kopf™) with a 37° C. heated pad. The PESCA library (AAV9, 1.9×10¹³ genome copies/mL) was stereotactically injected in V1 (800 nL per site at 25 nL/min) using a sharp glass pipette (25-45 μm diameter) that was left in place for 5 min prior to and 10 min following injection to minimize backflow. Two injections were performed per animal at coordinates 3.0 and 3.7 mm posterior, 2.5 mm lateral relative to bregma, and 0.6 mm ventral relative to the brain surface.

Individual rAAV-GRE constructs were stereotactically injected at a titer of 1×10¹¹ genome copies/mL. (250 nL per site at 25 nL/min). All injections were performed at two depths (0.4 and 0.7 mm ventral relative to the brain surface) to achieve broader infection across cortical layers. The injection coordinates relative to bregma were 3.0 or 3.7 mm posterior, 2.5 or −2.5 mm lateral.

Nuclear isolation: Single-nuclei suspensions were generated as described previously (see e.g., Mo et al., 2015, supra), with minor modifications. V1 was dissected and placed into a Dounce with homogenization buffer (0.25 M sucrose, 25 mM KCl, 5 mM MgCl₂, 20 mM Tricine-KOH, pH 7.8, 1 mM DTT, 0.15 mM spermine, 0.5 mM spermidine, protease inhibitors). The sample was homogenized using a tight pestle with 10 stokes. IGEPAL solution (5%, Sigma™) was added to a final concentration of 0.32%, and five additional strokes were performed. The homogenate was filtered through a 40 μm filter, and OptiPrep™ (Sigma™) added to a final concentration of 25% iodixanol. The sample was layered onto an iodixanol gradient and centrifuged at 10,000 g for 18 min as previously described (see e.g., Mo et al., 2015, supra; Stroud et al., 2017, supra). Nuclei were collected between the 30% and 40% iodixanol layers and diluted to 80,000-100,000 nuclei/mL for encapsulation. All buffers contained 0.15% RNasin Plus RNase Inhibitor (Promega™) and 0.04% BSA.

snRNA-Seq library preparation and sequencing: Single nuclei were captured and barcoded whole-transcriptome libraries prepared using the inDrops™ platform as previously described (see e.g., Klein et al., 2015, supra; Zilionis et al., 2017, supra), collecting five libraries of approximately 3000 nuclei from each animal. Briefly, single nuclei along with single primer-carrying hydrogels were captured into droplets using a microfluidic platform. Each hydrogel carried oligodT primers with a unique cell-barcode. Nuclei were lysed and the cell-barcode containing primers released from the hydrogel, initiating reverse transcription and barcoding of all cDNA in each droplet. Next, the emulsions were broken and cDNA across ˜3000 nuclei pooled into the same library. The cDNA was amplified by second strand synthesis and in vitro transcription, generating an amplified RNA intermediate which was fragmented and reverse transcribed into an amplified cDNA library.

For enrichment of virally-derived transcripts, a fraction (3 μL) of the amplified RNA intermediate was reverse transcribed with random hexamers without prior fragmentation. PCR was next used to amplify virally derived transcripts. The forward primer was designed to introduce the R1 sequence and anneal to a sequence uniquely present 5′ of the viral-barcode sequence present in the viral transcripts (SEQ ID NO: 6—5′-GCATCGATACCGAGCGC). The reverse primer was designed to anneal to a sequence present 5′ of the cell-barcode (SEQ ID NO: 7—5′-GGGTGTCGGGTGCAG). The result of the PCR is preferential amplification of the viral-derived transcripts, while simultaneously retaining the cell-barcode sequence necessary to assign each transcript to a particular cell/nucleus. Following PCR amplification (e.g., 18 cycles, Hot Start High-Fidelity Q5™ polymerase) all the libraries were indexed, pooled, and sequenced on a Nextseq 500™ benchtop DNA sequencer (Illumina™).

inDrop™ sample mapping and viral barcode deconvolution by cell: The published inDrops mapping pipeline (see e.g., available on the worldwide web at github.com/indrops/indrops) was used to assign reads to cells. To map viral sequences, a custom annotated transcriptome was generated using the indrops pipeline's build_index command supplied with two custom reference files: 1. the GRCm38.dna_sm.primary_assembly.fa fasta genome with an additional contig for each viral barcode (comprising 5′ sequence [SEQ ID NO: 8-gcatcgataccgagcgcgcgatcgc], barcode, and 3′ sequence [SEQ ID NO: 9-tcgagagatctgtgatagcggc]) and 2. a GTF annotation file, with all viral sequences assigned the same gene_id and gene_name, but unique transcript_id, transcript_name, and protein_id. After inDrops™ pipeline mapping and cell deconvolution, the pysam package was used to extract the ‘XB’ and ‘XU’ tags, which contain cell barcode and UMI sequences, respectively, from every read that mapped uniquely to any one of the custom viral contigs (i.e. requiring the read map to the 10 bp barcode with at most one mismatch) in the inDrops pipeline-output bam files. These barcode-UMI combinations were condensed to generate a final cell×GRE barcode UMI counts table for each sample.

Embedding and identification of cell types: Data from all nuclei (two animals, 5 libraries of ˜3000 nuclei per animal) were analyzed simultaneously. Viral-derived sequences were removed for the purposes of embedding clustering and cell type identification. The initial dataset contained 32,335 nuclei, with more than 200 unique non-viral transcripts (UMIs) assigned to each nucleus. An average of 866 unique non-viral transcripts was recovered per nucleus, representing 610 unique genes. The R software package Seurat (see e.g., Butler et al., 2018, Nature Biotechnology 36:411-420; Satija et al., 2015, Nature Biotechnology 33:495-502) was used to cluster cells. First, the data were log-normalized and scaled to 10,000 transcripts per cell. Variable genes were identified using the FindVariableGenes( ) function. The following parameters were used to set the minimum and maximum average expression and the minimum dispersion: x.low.cutoff=0.0125, x.high.cutoff=3, y.cutoff=0.5. Next, the data was scaled using the ScaleData( ) function, and principle component analysis (PCA) was carried out. The FindClusters( ) function using the top 30 principal components (PCs) and a resolution of 1.5 was used to determine the initial 29 clusters. Based on the expression of known marker genes, clusters were merged that represented the same cell type. The final list of cell types was: Excitatory neurons, PV Interneurons, SST Interneurons, VIP interneurons, NPY Interneurons, Astrocytes, Vascular-associated cells, Microglia, Oligodendrocytes, and Oligodendrocyte precursor cells.

Enrichment calculation: Viral vector expression for each of the 861 barcodes across the ten cell types was calculated by averaging the expression of barcoded transcripts across all the individual nuclei that were assigned to that cell type. The relative fold-enrichment in expression toward Sst+ cells was computed as the ratio of the mean expression in Sst+ cells and the mean expression in Sst− cells: (mean(Sst+ cells)+0.01)/(mean(Sst− cells)+0.01).

Viral GRE expression for each of the 287 barcodes was calculated at the single-nucleus level as a sum of the expression of the three barcodes that were paired with that GRE. Average GRE-driven expression across the ten cell types was calculated by averaging the expression of the GRE transcripts across all the individual nuclei that were assigned to that cell type. The relative fold-enrichment in GRE expression toward Sst+ cells was determined as the ratio of the mean expression in Sst+ cells and the mean expression in Sst− cells: (mean(Sst+ cells)+0.01)/(mean(Sst− cells)+0.01).

Differential gene expression: To identify which of the GRE-driven transcripts were statistically enriched in Sst+ vs. Sst− cells, differential gene expression analysis was carried out using the R package Monocle2 (see e.g., Trapnell et al., 2014, Nature Biotechnology 32.381-386). The data were modeled and normalized using a negative binomial distribution, consistent with snRNA-seq experiments. The functions estimateSizeFactors( ) estimateDispersions( ) and differentialGeneTest( ) were used to identify which of the GRE-derived transcripts were statistically enriched in Sst+ cells. GREs whose false discovery rate (FDR) was less than 0.01 were considered enriched.

Subsampling GRE reads: A matrix containing counts per cell for GRE12, GRE19, GRE22, GRE44, GRE80 was subsampled using the rbinom function from the ‘stats’ package in R with the following probabilities (0.5, 0.25, 0.125, 0.0625). The resulting matrix was then analyzed by differential gene expression using the R package Monocle2™ as stated above. This process was repeated ten times for each subsampling probability.

Fluorescence microscopy methods are described below.

Sample preparation: Mice were sacrificed and perfused with 4% PFA followed by PBS. The brain was dissected out of the skull and post-fixed with 4% PFA for 1-3 days at 4° C. The brain was mounted on the vibratome (Leica™ VT1000S) and coronally sectioned into 100 μm slices. Sections containing V1 were arrayed on glass slides and mounted using DAPI Fluoromount-G™ (Southern Biotech™).

Sample imaging: Sections containing V1 were imaged on a Leica™ SPE confocal microscope using an ACS APO 10×/0.30 CS objective. Tiled V1 cortical areas of ˜1.2 mm by ˜0.5 mm were imaged at a single optical section to avoid counting the same cell across multiple optical sections. Channels were imaged sequentially to avoid any optical crosstalk.

Immunostaining: To identify parvalbumin (PV)+ cells, coronal sections were washed three times with PBS containing 0.3% TritonX-100 (PBST) and blocked for 1 hr at room temperature with PBST containing 5% donkey serum. Section were incubated overnight at 4° C. with mouse anti-PVALB antibody 1:2000 (Millipore™), washed again three times with PBST, and incubated for 1 hr at room temperature with 1:500 donkey anti-mouse 647 secondary antibody (Life Technologies™). After washing in PBST and PBS, samples were mounted onto glass slides using DAPI Fluoromount-G™.

Quantification of the percentage of GFP+ cells that were SST+, VIP+, and PV+: Across all images, coordinates were registered for each GFP+ cell that could be visually discerned. An automated ImageJ™ script was developed to quantify the intensity of each acquired channel for a given GFP+ cell. A circular mask (radius=5.7 μm) was created at each coordinate representing a GFP-positive cell, background subtracted (rolling ball, radius=72 μm) each channel, and the mean signal of the masked area was quantified. To identify the threshold intensity used to classify each GFP+ cell as either SST+, VIP+ or PV+, the background signal was first determined in the channel representing SST, VIP or PV by selecting multiple points throughout the area visually identified as background. These background points were masked as small circular areas (e.g., radius=5.7 μm), over which the mean background signal was quantified. The highest mean background signal for SST, VIP and PV was conservatively chosen as the threshold for classifying GFP+ cells as SST+, VIP+ or PV+, respectively.

Quantification of the distribution of cells as a function of distance from pia: A semiautomated ImageJ™ algorithm was developed to trace the pia in each image, generate a Euclidean Distance Map (EDM), and calculate the distance from the pia to each GFP+ cell.

Quantification of the percentage of SST+ cells that were GFP+: An automated algorithm was developed to identify SST+ cells after appropriate background subtraction, image thresholding, masking and filtering for all objects of appropriate size and circularity. The number of SST+ objects (cells) was then counted within a minimal polygonal area that encompassed all GFP+ cells in that image. The ratio of the number of GFP+ cells and SST+ cells within the area of infection (herein identified as area with discernable GFP+ cells) was calculated.

Slice preparation: Acute, coronal brain slices containing visual cortex of 250-300 μm thickness were prepared using a sapphire blade (Delaware Diamond Knives™) and a VT1000S vibratome (Leica™). Mice were anesthetized though inhalation of isoflurane, then decapitated. The head was immediately immersed in an ice-cold solution containing (in mM): 130 K-gluconate, 15 KCl, 0.05 EGTA, 20 HEPES, and 25 glucose (pH 7.4 with NaOH; Sigma™). The brains were quickly dissected and cut in the same ice-cold, gluconate based solution while oxygenated with 95% O₂/5% CO₂. Slices then recovered at 32° C. for 20-30 min in oxygenated artificial cerebrospinal fluid (ACSF) in mM: 125 NaCl, 26 NaHCO₃, 1.25 NaH2PO4, 2.5 KCl, 1.0 MgCl2, 2.0 CaCl2, and 25 glucose (Sigma™), adjusted to 310-312 mOsm with water.

Electrophysiological recordings: Using an Olympus™ BX51WI microscope equipped with a 60× water immersion objective, fluorescence illumination was used to identify rAAV-GRE44-GFP⁺ (tdTomato+ red and GFP+ green) and rAAV-GRE44-GFP⁻ (only tdTomato+ red) SST neurons in the area of injection/AAV infection (see e.g., FIG. 32A-32D). rAAV-GRE44-GFP⁻ neurons were recorded if they were in the same field of view as rAAV-GRE44-GFP⁺ neurons under 60×. For rAAV-GRE12-Gq-DREADD-tdTomato experiments (see e.g., FIG. 3K-3M; see e.g., SEQ ID NO: 22), tdTomato⁺ cells and morphologically identified pyramidal neurons in the same field of view under 60× were recorded. Whole-cell current clamp recordings of these neurons in coronal visual cortex slices of P50 to P80 wild-type mice were performed using borosilicate glass pipettes (3-6 MOhms, Sutter Instrument™) filled with an internal solution (in mM): 116 KMeSO3, 6 KCl, 2 NaCl, 0.5 EGTA, 20 HEPES, 4 MgATP, 0.3 NaGTP, 10 NaPO₄ creatine (pH 7.25 with KOH; Sigma™). Neurobiotin (1.5%) was occasionally included in the internal solution to allow for post-hoc morphological reconstruction of recorded cells. All experiments were performed at room temperature in oxygenated ACSF. Series resistance was compensated by at least 60% in a voltage-clamp configuration before switching to current-clamp (‘I Clamp Normal’). After break-in, a systematic series of 1 s current injections ranging from ˜100 pA to 500 pA were applied to each cell using the User List function in the ‘Edit Waveform’ tab of pClamp. After such baseline firing rates were calculated, CNO (2 μM, Sigma™) was bath applied. An average of at least three trials for each current injection was calculated before and during CNO application.

Electrophysiological data acquisition and analysis: For electrophysiology, data acquisition of current-clamp experiments was performed using Clampex10.2™, an Axopatch 200B™ amplifier, filtered at 2 kHz and digitized at 20 kHz with a DigiData 1440™ data acquisition board (Molecular Devices™). Analysis of electrophysiological parameters was done using Clampfit™ (Molecular Devices™), Prism7™ (GraphPad Software™), Excel™ (Microsoft™), and custom software written in Igor Pro™ version 6.1.2.1 (WaveMetrics™). Membrane potentials in this study were not corrected for the liquid junction potential and are thus positively biased by 8 mV. For analysis of action potential waveform in FIG. 32A-32D and Table 4, the first action potential that appeared during a current injection equivalent to the rheobase was analyzed, as well as the first action potential of the subsequent two current injections. For example, if the rheobase were 20 pA, then all the parameters defined in the next section were also analyzed for the first action potential elicited with 20, 25, and 30 pA of injected current, and averaged.

Definitions of electrophysiological parameters as used here are recited below.

As used herein, AP Height (in millivolts) is defined as the difference between the peak of the action potential and the most negative voltage during the afterhyperpolarization immediately following the spike.

As used herein, AP Peak (in millivolts) is defined as the most depolarized (positive) potential of the spike.

As used herein, AP Trough (in millivolts) is defined as the most negative voltage reached during the afterhyperpolarization immediately following the spike.

As used herein, Fₘₐₓ initial (in Hertz) is defined as the average of the reciprocal of the first three interstimulus intervals, measured at the maximal current step injected before spike inactivation.

As used herein, Fₘₐₓ steady-state (in Hertz) is defined as the average of the reciprocal of the last three interstimulus intervals, measured at the maximal current step injected before spike inactivation.

As used herein, rate of rise (in volts per second) is defined as maximal voltage slope (dV/dt) during the upstroke (rising phase) of the action potential.

As used herein, rheobase (in picoamperes) is defined as the minimal 1000 ms current step (in increments of 5 pA) needed to elicit an action potential.

As used herein, Rᵢₙ (in megaohms, MΩ) is defined as input resistance, determined by using Ohm's law to measure the change in voltage in response to a −50 pA, 1000 ms hyperpolarizing current at rest.

As used herein, spike adaptation ratio is defined as the ratio of Fₘₐₓ steady-state to Fₘₐₓ initial.

As used herein, spike width (in milliseconds, used interchangeably with spike half-width) is defined as the width at half-maximal spike height as defined above.

As used herein, τₘ (in milliseconds) is defined as membrane time constant, determined by fitting a mono-exponential curve to the voltage chance in response to a −50 pA, 1000 ms hyperpolarizing current at rest.

As used herein, threshold (in millivolts) is defined as the membrane potential at which dV/dt=5 V/s.

As used herein, Vᵣₑₛₜ (in millivolts) is defined as resting membrane potential a few minutes after breaking in without any current injection.

Sequences

SEQ ID NO: 10-pAAV-ΔGRE-GFP; italicized bases denote ITRs; bold bases denote eGFP
AACTACTTACTCTAGCTTCCCGGCAACAATTAATAGACTGGATGGAGGCGGATAAAGTTGCA
GGACCACTTCTGCGCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCGCTGAG
ATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTC
ATGACCAAAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAA
CTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACCGGATAA
GGCGCAGCGGTCGGGCTGAACGGGGGGTTCGTGCACACAGCCCAGCTTGGAGCGAACGACC
TACACCGAACTGAGATACCTACAGCGTGAGCTATGAGAAAGCGCCACGCTTCCCGAAGGGA
GAAAGGCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTCCTGCAGGCAGCTGCGCGCT
CGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTGGCCAACTCCATCACTAGGGGTTCCTGCGGCC
GCACGCGTTTAATGTCGACTGATATCGAATTCCTGCAGCCCGGGCTGGGCATAAAAGTCAGG
GCAGAGCCATCTATTGCTTACATTTGCTTCTAGCCTGCAGGTCGAGGAGCGCAGCCTTCCAG
AAGCAGAGCGCGGCGCCTTAAGCTGCAGAAGTTGGTCGTGAGGCACTGGGCAGGTAAGTAT
CAAGGTTACAAGACAGGTTTAAGGAGACCAATAGAAACTGGGCTTGTCGAGACAGAGAAGA
CTCTTGCGTTTCTGATAGGCACCTATTGGTCTTACTGACATCCACTTTGCCTTTCTCTCCACAG
GTGTCCACTCCCAGTTCAATTACAGCTCTTAAGAAACTAGTAGCCACCATGGTGAGCAAGG
GCGAGGAGCTGTTCACCGGGGTGGTGCCCATCCTGGTCGAGCTGGACGGCGACGTAA
ACGGCCACAAGTTCAGCGTGTCCGGCGAGGGCGAGGGCGATGCCACCTACGGCAAGC
TGACCCTGAAGTTCATCTGCACCACCGGCAAGCTGCCCGTGCCCTGGCCCACCCTCGT
GACCACCCTGACCTACGGCGTGCAGTGCTTCAGCCGCTACCCCGACCACATGAAGCAG
CACGACTTCTTCAAGTCCGCCATGCCCGAAGGCTACGTCCAGGAGCGCACCATCTTCTTCA
AGGACGACGGCAACTACAAGACCCGCGCCGAGGTGAAGTTCGAGGGCGACACCCTGGTGAA
CCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCA
AGGTGAACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTACCTGAGC
ACCCAGTCCGCCCTGAGCAAAGACCCCAACGAGAAGCGCGATCACATGGTCCTGCTGGAGT
TCGTGACCGCCGCCGGGATCACTCTCGGCATGGACGAGCTGTACAAGTAAAGGCGCGCCAC
CCCTGCAGGGAATTCCCCCTGCAGGGAATTCGATATCAAGCTTATCGATAATCAACCTCTGG
ATTACAAAATTTGTGAAAGATTGACTGGTATTCTTAACTATGTTGCTCCTTTTACGCTATGTG
GATACGCTGCTTTAATGCCTTTGTATCATGCTATTGCTTCCCGTATGGCTTTCATTTTCTCCTC
CTTGTATAAATCCTGGTTGCTGTCTCTTTATGAGGAGTTGTGGCCCGTTGTCAGGCAACGTGG
CGTGGTGTGCACTGTGTTTGCTGACGCAACCCCCACTGGTTGGGGCATTGCCACCACCTGTC
AGCTCCTTTCCGGGACTTTCGCTTTCCCCCTCCCTATTGCCACGGCGGAACTCATCGCCGCCT
GCCTTGCCCGCTGCTGGACAGGGGCTCGGCTGTTGGGCACTGACAATTCCGTGGTGTTGTCG
GGGAAATCATCGTCCTTTCCTTGGCTGCTCGCCTATGTTGCCACCTGGATTCTGCGCGGGACG
TCCTTCTGCTACGTCCCTTCGGCCCTCAATCCAGCGGACCTTCCTTCCCGCGGCCTGCTGCCG
GCTCTGCGGCCTCTTCCGCGTCTTCGCCTTCGCCCTCAGACGAGTCGGATCTCCCTTTGGGCC
GCCTCCCCGCATCGATACCGAGCGCGCGATCGCAAACAAACCTCGAGAGATCTGTGATAGC
GGCCATCAAGCTGGCCGCGACTCTAGATCATAATCAGCCATACCACATTTGTAGAGGTTTTA
CTTGCTTTAAAAAACCTCCCACACCTCCCCCTGAACCTGAAACATAAAATGAATGCAATTGT
TGTTGTTAACTTGTTTATTGCAGCTTATAATGGTTACAAATAAAGCAATAGCATCACAAATTT
CACAAATAAAGCATTTTTTTCACTGCATTCTAGTTGTGGTTTGTCCAAACTCATCAATGTATC
AGCTTATCGATACCGCATGCACGTGCGGACCGAGCGGCCGCAGGAACCCCTAGTGATGGAGTT
GGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGCTGCCTGCAGGGGCGCCTG
ATGCGGTATTTTCTCCTTACGCATCTGTGCGGTATTTCACACCGCATACGTCAAAGCAACCAT
AGTACGCGCCCTGTAGCGGCGCATTAAGCGCGGCGGGTGTGGTGGTTACGCGCAGCGTGAC
CGCTACACTTGCCAGCGCCCTAGCGCCCGCTCCTTTCGCTTTCTTCCCTTCCTTTCTCGCCACG
TTCGCCGGCTTTCCCCGTCAAGCTCTAAATCGGGGGCTCCCTTTAGGGTTCCGATTTAGTGCT
TTACGGCACCTCGACCCCAAAAAACTTGATTTGGGTGATGGTTCACGTAGTGGGCCATCGCC
CTGATAGACGGTTTTTCGCCCTTTGACGTTGGAGTCCACGTTCTTTAATAGTGGACTCTTGTT
CCAAACTGGAACAACACTCAACCCTATCTCGGGCTATTCTTTTGATTTATAAGGGATTTTGCC
GATTTCGGCCTATTGGTTAAAAAATGAGCTGATTTAACAAAAATTTAACGCGAATTTTAACA
AAATATTAACGTTTACAATTTTATGGTGCACTCTCAGTACAATCTGCTCTGATGCCGCATAGT
TAAGCCAGCCCCGACACCCGCCAACACCCGCTGACGCGCCCTGACGGGCTTGTCTGCTCCCG
GCATCCGCTTACAGACAAGCTGTGACCGTCTCCGGGAGCTGCATGTGTCAGAGGTTTTCACC
GTCATCACCGAAACGCGCGAGACGAAAGGGCCTCGTGATACGCCTATTTTTATAGGTTAATG
TCATGATAATAATGGTTTCTTAGACGTCAGGTGGCACTTTTCGGGGAAATGTGCGCGGAACC
CCTATTTGTTTATTTTTCTAAATACATTCAAATATGTATCCGCTCATGAGACAATAACCCTGA
TAAATGCTTCAATAATATTGAAAAAGGAAGAGTATGAGTATTCAACATTTCCGTGTCGCCCT
TATTCCCTTTTTTGCGGCATTTTGCCTTCCTGTTTTTGCTCACCCAGAAACGCTGGTGAAAGT
AAAAGATGCTGAAGATCAGTTGGGTGCACGAGTGGGTTACATCGAACTGGATCTCAACAGC
GGTAAGATCCTTGAGAGTTTTCGCCCCGAAGAACGTTTTCCAATGATGAGCACTTTTAAAGT
TCTGCTATGTGGCGCGGTATTATCCCGTATTGACGCCGGGCAAGAGCAACTCGGTCGCCGCA
TACACTATTCTCAGAATGACTTGGTTGAGTACTCACCAGTCACAGAAAAGCATCTTACGGAT
GGCATGACAGTAAGAGAATTATGCAGTGCTGCCATAACCATGAGTGATAACACTGCGGCCA
ACTTACTTCTGACAACGATCGGAGGACCGAAGGAGCTAACCGCTTTTTTGCACAACATGGGG
GATCATGTAACTCGCCTTGATCGTTGGGAACCGGAGCTGAATGAAGCCATACCAAACGACG
AGCGTGACACCACGATGCCTGTAGCAATGGCAACAACGTTGCGCAAACTATTAACTGGCG
SEQ ID NO: 11-pAAV-GRE12-GFP; italicized bases denote ITRs; bold bases denote
eGFP; GRE12 comprises bold underlined bases (see e.g., SEQ ID NO: 14, SEQ ID NO: 17)
AACTACTTACTCTAGCTTCCCGGCAACAATTAATAGACTGGATGGAGGCGGATAAAGTTGCA
GGACCACTTCTGCGCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCGCTGAG
ATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTC
ATGACCAAAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAA
CTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACCGGATAA
GGCGCAGCGGTCGGGCTGAACGGGGGGTTCGTGCACACAGCCCAGCTTGGAGCGAACGACC
TACACCGAACTGAGATACCTACAGCGTGAGCTATGAGAAAGCGCCACGCTTCCCGAAGGGA
GAAAGGCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTCCTGCAGGCAGCTGCGCGCT
CGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTGGCCAACTCCATCACTAGGGGTTCCTGCGGCC
GCACGCGTTTAATCTTTAGAGGGGGAAACTGCCTTTTGAGTTGTTTATATATAAAGTTAT
TTAAATAATGAAGATCATTTTTTTCTGCCTATAATGTTTTTCTTGAGATGATGCTTTCTT
GAAAAAAATATTTTCAAAGGCTGAAAACAAATACATAAGAACTCAGTAAACTCGGGAA
GTGTTTAGCTTCATAATCAGACTGTGCAGAAGATAGGAAGCAGCAGCCGGATCCACAG
CCTCTGATTGTCCCAAATCACAGGAGTCATCAACTGAGTACTCCAAAAAGGAAAACAAGC
CATTTTCAGCTAAAAGATATGAGCATAATGTGTACCATAATCTCACAGTGGCTGTTTTAGAA
CCAAGAGTGTTTGTGACTTAATTTGAATTTCTCAATGCAACATTTCTCAAAAATTCCTTAAAC
GTCATGTCATAGATGATTTATTATGTACAAAACATAACTGTTGAGAAACTCCATTTCCTTGCC
TTCTGGGAGGAACCTTAGGAAACATCAGCAGCAGGTGCAAAGTATTCCATAGAGAGAGGGC
TGGCATAAAGAACATATTTATTCATCAGTTCCAAATTTCCCTGCTTCTGAGGGCTTAAAAAG
AGGGATTTCTTGAGCTGAGGAAATTAAAAACAAAACAAACAACTATGCTGAAAGAGGACTA
GAAATGTTCTGGGATATTGTGAAATCTAGACTTGAAATTCCTTCTCATTTCCTTATGCACAGA
TTTTAACACCCTTGGTTTCTTCGGAGTAGTCGACTGATATCGAATTCCTGCAGCCCGGGCTGG
GCATAAAAGTCAGGGCAGAGCCATCTATTGCTTACATTTGCTTCTAGCCTGCAGGTCGAGGA
GCGCAGCCTTCCAGAAGCAGAGCGCGGCGCCTTAAGCTGCAGAAGTTGGTCGTGAGGCACT
GGGCAGGTAAGTATCAAGGTTACAAGACAGGTTTAAGGAGACCAATAGAAACTGGGCTTGT
CGAGACAGAGAAGACTCTTGCGTTTCTGATAGGCACCTATTGGTCTTACTGACATCCACTTT
GCCTTTCTCTCCACAGGTGTCCACTCCCAGTTCAATTACAGCTCTTAAGAAACTAGTAGCCAC
CATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCC
ACCTACGGCAAGCTGACCCTGAAGTTCATCTGCACCACCGGCAAGCTGCCCGTGCCCT
GGCCCACCCTCGTGACCACCCTGACCTACGGCGTGCAGTGCTTCAGCCGCTACCCCGA
CCACATGAAGCAGCACGACTTCTTCAAGTCCGCCATGCCCGAAGGCTACGTCCAGGAGCG
CACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCC
TGGGGCACAAGCTGGAGTACAACTACAACAGCCACAACGTCTATATCATGGCCGACAAGCA
GAAGAACGGCATCAAGGTGAACTTCAAGATCCGCCACAACATCGAGGACGGCAGCGTGCAG
CTCGCCGACCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGATCACATG
GTCCTGCTGGAGTTCGTGACCGCCGCCGGGATCACTCTCGGCATGGACGAGCTGTACAAGTA
AAGGCGCGCCACCCCTGCAGGGAATTCCCCCTGCAGGGAATTCGATATCAAGCTTATCGATA
ATCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCTTT
CATTTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATTGC
CACCACCTGTCAGCTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTATGTTGCCACCTGGATTC
TGCGCGGGACGTCCTTCTGCTACGTCCCTTCGGCCCTCAATCCAGCGGACCTTCCTTCCCGCG
GCCTGCTGCCGGCTCTGCGGCCTCTTCCGCGTCTTCGCCTTCGCCCTCAGACGAGTCGGATCT
CCCTTTGGGCCGCCTCCCCGCATCGATACCGAGCGCGCGATCGCAAACAAACCTCGAGAGAT
CTGTGATAGCGGCCATCAAGCTGGCCGCGACTCTAGATCATAATCAGCCATACCACATTTGT
AGAGGTTTTACTTGCTTTAAAAAACCTCCCACACCTCCCCCTGAACCTGAAACATAAAATGA
ATGCAATTGTTGTTGTTAACTTGTTTATTGCAGCTTATAATGGTTACAAATAAAGCAATAGCA
TCACAAATTTCACAAATAAAGCATTTTTTTCACTGCATTCTAGTTGTGGTTTGTCCAAACTCA
TCAATGTATCAGCTTATCGATACCGCATGCACGTGCGGACCGAGCGGCCGCAGGAACCCCTA
GTGATGGAGTTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCAGCTGCCTGC
AGGGGCGCCTGATGCGGTATTTTCTCCTTACGCATCTGTGCGGTATTTCACACCGCATACGTC
AAAGCAACCATAGTACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCTCCCTTTAGGGTTC
CGATTTAGTGCTTTACGGCACCTCGACCCCAAAAAACTTGATTTGGGTGATGGTTCACGTAG
TGGGCCATCGCCCTGATAGACGGTTTTTCGCCCTTTGACGTTGGAGTCCACGTTCTTTAATAG
TGGACTCTTGTTCCAAACTGGAACAACACTCAACCCTATCTCGGGCTATTCTTTTGATTTATA
AGGGATTTTGCCGATTTCGGCCTATTGGTTAAAAAATGAGCTGATTTAACAAAAATTTAACG
CGAATTTTAACAAAATATTAACGTTTACAATTTTATGGTGCACTCTCAGTACAATCTGCTCTG
ATGCCGCATAGTTAAGCCAGCCCCGACACCCGCCAACACCCGCTGACGCGCCCTGACGGGCT
TGTCTGCTCCCGGCATCCGCTTACAGACAAGCTGTGACCGTCTCCGGGAGCTGCATGTGTCA
GAGGTTTTCACCGTCATCACCGAAACGCGCGAGACGAAAGGGCCTCGTGATACGCCTATTTT
TATAGGTTAATGTCATGATAATAATGGTTTCTTAGACGTCAGGTGGCACTTTTCGGGGAAAT
GTGCGCGGAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAATGATGAGC
ACTTTTAAAGTTCTGCTATGTGGCGCGGTATTATCCCGTATTGACGCCGGGCAAGAGCAACT
CGGTCGCCGCATACACTATTCTCAGAATGACTTGGTTGAGTACTCACCAGTCACAGAAAAGC
ATCTTACGGATGGCATGACAGTAAGAGAATTATGCAGTGCTGCCATAACCATGAGTGATAAC
ACTGCGGCCAACTTACTTCTGACAACGATCGGAGGACCGAAGGAGCTAACCGCTTTTTTGCA
CAACATGGGGGATCATGTAACTCGCCTTGATCGTTGGGAACCGGAGCTGAATGAAGCCATA
CCAAACGACGAGCGTGACACCACGATGCCTGTAGCAATGGCAACAACGTTGCGCAAACTAT
TAACTGGCG
SEQ ID NO: 12-pAAV-GRE22-GFP; italicized bases denote ITRs; bold bases denote
eGFP; GRE22 comprises bold underlined bases (see e.g., SEQ ID NO: 15, SEQ ID NO: 18)
AACTACTTACTCTAGCTTCCCGGCAACAATTAATAGACTGGATGGAGGCGGATAAAGTTGCA
GGACCACTTCTGCGCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCGCTGAG
ATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTC
ATGACCAAAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAA
CTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACCGGATAA
GGCGCAGCGGTCGGGCTGAACGGGGGGTTCGTGCACACAGCCCAGCTTGGAGCGAACGACC
TACACCGAACTGAGATACCTACAGCGTGAGCTATGAGAAAGCGCCACGCTTCCCGAAGGGA
GAAAGGCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTCCTGCAGGCAGCTGCGCGCT
CGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTGGCCAACTCCATCACTAGGGGTTCCTGCGGCC
GCACGCGTTTAATAGCCAGGACTACACAGAGAAACCCTGTCTCAAAAAAACAAAATCAA
AACAAAACAAACAAACAAAAAAGCTAATGACTCCATCATGACTGTAACAAACACATCAG
TGCGGCAGTGAGAGCCCGTCTGTCAGCATCAGCAACAGCATTAGTCAGACTGTATTTG
TGAGCATATTTGCTTAGGTCTCTTCTAAATACCCTTCACTTTTCTCTCAGAGAAACCCA
GTTCATCGTATTCTGAAAAGGAGCGGCCGTAAAGGACTGATCCTGTCTGAAGCACTTTGG
TATAAAAGTTGCTTAGCAGTGGGGCAGAAAAGAAAAAAAGCAATTAAGTTTATATTTAGTG
ATCTATCTATACACATCTGGAGCACATTTGGGAAAGAATTCAAAAGGGCCAATTCATTGCAT
GCCTCCTGCTACAGAACGAGTGTGGGAGTCAAGCTGCGATTTCCACAGCATCAGACATTTAT
TGTTGACTTCAAAAAGTTCTCCCACTTATGTGTAATTACTATCCTAGCAAATGGCTCTGAAAT
TTCAGCTTCTTAAGCATAAGGCAGAGTGGTCCTTTAAAAGTAAAATAAAACGTAGGCCCTAT
GAGATAAAATTAAGATAAATTAAGAATCAGTTACTTCCAAGACGAAGCACTTATGGTGCAT
GCCTTCTTATATAAAGCAGATCCTTACCATGTATGTGTGCTGTTTGCTTGCCAAGACCAAGAT
GTCTGTCGACTGATATCGAATTCCTGCAGCCCGGGCTGGGCATAAAAGTCAGGGCAGAGCC
ATCTATTGCTTACATTTGCTTCTAGCCTGCAGGTCGAGGAGCGCAGCCTTCCAGAAGCAGAG
CGCGGCGCCTTAAGCTGCAGAAGTTGGTCGTGAGGCACTGGGCAGGTAAGTATCAAGGTTA
CAAGACAGGTTTAAGGAGACCAATAGAAACTGGGCTTGTCGAGACAGAGAAGACTCTTGCG
TTTCTGATAGGCACCTATTGGTCTTACTGACATCCACTTTGCCTTTCTCTCCACAGGTGTCCA
CTCCCAGTTCAATTACAGCTCTTAAGAAACTAGTAGCCACCATGGTGAGCAAGGGCGAGGA
GCTGTTCACCGGGGTGGTGCCCATCCTGGTCGAGCTGGACGGCGACGTAAACGGCCAC
AAGTTCAGCGTGTCCGGCGAGGGCGAGGGCGATGCCACCTACGGCAAGCTGACCCTG
AAGTTCATCTGCACCACCGGCAAGCTGCCCGTGCCCTGGCCCACCCTCGTGACCACCC
TGACCTACGGCGTGCAGTGCTTCAGCCGCTACCCCGACCACATGAAGCAGCACGACTT
CTTCAAGTCCGCCATGCCCGAAGGCTACGTCCAGGAGCGCACCATCTTCTTCAAGGACGAC
GGCAACTACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGAGTACAA
CTACAACAGCCACAACGTCTATATCATGGCCGACAAGCAGAAGAACGGCATCAAGGTGAAC
TTCAAGATCCGCCACAACATCGAGGACGGCAGCGTGCAGCTCGCCGACCACTACCAGCAGA
ACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCG
CCGCCGGGATCACTCTCGGCATGGACGAGCTGTACAAGTAAAGGCGCGCCACCCCTGCAGG
GAATTCCCCCTGCAGGGAATTCGATATCAAGCTTATCGATAATCAACCTCTGGATTACAAAA
TTTGTGAAAGATTGACTGGTATTCTTAACTATGTTGCTCCTTTTACGCTATGTGGATACGCTG
CTTTAATGCCTTTGTATCATGCTATTGCTTCCCGTATGGCTTTCATTTTCTCCTCCTTGTATAA
ATCCTGGTTGCTGTCTCTTTATGAGGAGTTGTGGCCCGTTGTCAGGCAACGTGGCGTGGTGTG
CACTGTGTTTGCTGACGCAACCCCCACTGGTTGGGGCATTGCCACCACCTGTCAGCTCCTTTC
CGGGACTTTCGCTTTCCCCCTCCCTATTGCCACGGCGGAACTCATCGCCGCCTGCCTTGCCCG
CTGCTGGACAGGGGCTCGGCTGTTGGGCACTGACAATTCCGTGGTGTTGTCGGGGAAATCAT
CGTCCTTTCCTTGGCTGCTCGCCTATGTTGCCACCTGGATTCTGCGCGGGACGTCCTTCTGCT
ACGTCCCTTCGGCCCTCAATCCAGCGGACCTTCCTTCCCGCGGCCTGCTGCCGGCTCTGCGGC
CTCTTCCGCGTCTTCGCCTTCGCCCTCAGACGAGTCGGATCTCCCTTTGGGCCGCCTCCCCGC
ATCGATACCGAGCGCGCGATCGCAAACAAACCTCGAGAGATCTGTGATAGCGGCCATCAAG
CTGGCCGCGACTCTAGATCATAATCAGCCATACCACATTTGTAGAGGTTTTACTTGCTTTAAA
AAACCTCCCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCACAAATAAA
GCATTTTTTTCACTGCATTCTAGTTGTGGTTTGTCCAAACTCATCAATGTATCAGCTTATCGAT
ACCGCATGCACGTGCGGACCGAGCGGCCGCAGGAACCCCTAGTGATGGAGTTGGCCACTCCCT
CTCTGCGCGCTCGCTCGCTCACTGAGGCCGGGCGACCAAAGGTCGCCCGACGCCCGGGCTTTGC
CCGGGCGGCCTCAGTGAGCGAGCGAGCGCGCAGCTGCCTGCAGGGGCGCCTGATGCGGTATTT
TCTCCTTACGCATCTGTGCGGTATTTCACACCGCATACGTCAAAGCAACCATAGTACGCGCC
CTGTAGCGGCGCATTAAGCGCGGCGGGTGTGGTGGTTACGCGCAGCGTGACCGCTACACTTG
CCAGCGCCCTAGCGCCCGCTCCTTTCGCTTTCTTCCCTTCCTTTCTCGCCACGTTCGCCGGCTT
TCCCCGTCAAGCTCTAAATCGGGGGCTCCCTTTAGGGTTCCGATTTAGTGCTTTACGGCACCT
CGACCCCAAAAAACTTGATTTGGGTGATGGTTCACGTAGTGGGCCATCGCCCTGATAGACGG
TTTTTCGCCCTTTGACGTTGGAGTCCACGTTCTTTAATAGTGGACTCTTGTTCCAAACTGGAA
CAACACTCAACCCTATCTCGGGCTATTCTTTTGATTTATAAGGGATTTTGCCGATTTCGGCCT
ATTGGTTAAAAAATGAGCTGATTTAACAAAAATTTAACGCGAATTTTAACAAAATATTAACG
TTTACAATTTTATGGTGCACTCTCAGTACAATCTGCTCTGATGCCGCATAGTTAAGCCAGCCC
CGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGA
AACGCGCGAGACGAAAGGGCCTCGTGATACGCCTATTTTTATAGGTTAATGTCATGATAATA
ATGGTTTCTTAGACGTCAGGTGGCACTTTTCGGGGAAATGTGCGCGGAACCCCTATTTGTTTA
TTTTTCTAAATACATTCAAATATGTATCCGCTCATGAGACAATAACCCTGATAAATGCTTCAA
TAATATTGAAAAAGGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGTAAAAGATGCTGA
AGATCAGTTGGGTGCACGAGTGGGTTACATCGAACTGGATCTCAACAGCGGTAAGATCCTTG
AGAGTTTTCGCCCCGAAGAACGTTTTCCAATGATGAGCACTTTTAAAGTTCTGCTATGTGGC
GCGGTATTATCCCGTATTGACGCCGGGCAAGAGCAACTCGGTCGCCGCATACACTATTCTCA
GAATGACTTGGTTGAGTACTCACCAGTCACAGAAAAGCATCTTACGGATGGCATGACAGTA
AGAGAATTATGCAGTGCTGCCATAACCATGAGTGATAACACTGCGGCCAACTTACTTCTGAC
AACGATCGGAGGACCGAAGGAGCTAACCGCTTTTTTGCACAACATGGGGGATCATGTAACT
CGCCTTGATCGTTGGGAACCGGAGCTGAATGAAGCCATACCAAACGACGAGCGTGACACCA
CGATGCCTGTAGCAATGGCAACAACGTTGCGCAAACTATTAACTGGCG
SEQ ID NO: 13-pAAV-GRE44-GFP; italicized bases denote ITRs; bold bases denote
eGFP; GRE44 comprises bold underlined bases (see e.g., SEQ ID NO: 16, SEQ ID NO: 19)
AACTACTTACTCTAGCTTCCCGGCAACAATTAATAGACTGGATGGAGGCGGATAAAGTTGCA
GGACCACTTCTGCGCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCGCTGAG
ATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTC
ATGACCAAAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAA
CTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACCGGATAA
GGCGCAGCGGTCGGGCTGAACGGGGGGTTCGTGCACACAGCCCAGCTTGGAGCGAACGACC
TACACCGAACTGAGATACCTACAGCGTGAGCTATGAGAAAGCGCCACGCTTCCCGAAGGGA
GAAAGGCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTCCTGCAGGCAGCTGCGCGCT
CGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTGGCCAACTCCATCACTAGGGGTTCCTGCGGCC
GCACGCGTTTAATGTTCAGTACCCAGACACTCCCATACCCTTATTTAGAAGAAATAAATA
TCATCAAGTCATAATATCCTTGACTGATTAAGAAAGCCACTTTGTAAGTGTTTATTAAA
CTGTCAAGAAACTTACAGAATTTACTACATGATCGTTAGAATAACTTTGAGTCAGGACA
TATTTGATATGACTTAATCATACTCCCTCCAAAAGGAAATAAGGCTTTGTGAAGGTAAA
TTATTTCTTCCTGGGTTGGATATGTGTTTATGGAGTGATCATTCAGCTGTTCCCAACCTIC
ATTCTGAAAAGGCCTCAGAACACTTCATGATGAATCAAGCTGTATCCTGAATAGAGTAAAAT
GAACCACTTCGTAGGAACTATGGTGTCACCACATCAGCAATTCTTATTGAAAAGTGTGCATT
TCTTATTCACATATTTCAAAGATGGTATTCCAGAGGAGTGATTTTCTCAATGTATTTTTCATC
TACAAGCCTTCATTTTAAGCCTACCACCGTGTGTGTTTTCAAGACAGCAATTATCGTTTTAAA
ATGTGCAGGTCTAGCTTGAGCTTCTCAGCAAGTTTCTATGCCAAAGAAAACACCAATCCTTT
CCATTTACTGAGAATCAATGTTTAATCCTCCTTTTTGTTCTCATACTTATTACAAATCATAAA
GAATTCTGAGTGTCAGTTTGATAACTAGAAGCTCCATGTACCATTCCTGCTCCTTATTGAGTC
GACTGATATCGAATTCCTGCAGCCCGGGCTGGGCATAAAAGTCAGGGCAGAGCCATCTATTG
CTTACATTTGCTTCTAGCCTGCAGGTCGAGGAGCGCAGCCTTCCAGAAGCAGAGCGCGGCGC
CTTAAGCTGCAGAAGTTGGTCGTGAGGCACTGGGCAGGTAAGTATCAAGGTTACAAGACAG
GTTTAAGGAGACCAATAGAAACTGGGCTTGTCGAGACAGAGAAGACTCTTGCGTTTCTGATA
GGCACCTATTGGTCTTACTGACATCCACTTTGCCTTTCTCTCCACAGGTGTCCACTCCCAGTT
CAATTACAGCTCTTAAGAAACTAGTAGCCACCATGGTGAGCAAGGGCGAGGAGCTGTTCA
CCGGGGTGGTGCCCATCCTGGTCGAGCTGGACGGCGACGTAAACGGCCACAAGTTCA
GCGTGTCCGGCGAGGGCGAGGGCGATGCCACCTACGGCAAGCTGACCCTGAAGTTCA
TCTGCACCACCGGCAAGCTGCCCGTGCCCTGGCCCACCCTCGTGACCACCCTGACCTA
CGGCGTGCAGTGCTTCAGCCGCTACCCCGACCACATGAAGCAGCACGACTTCTTCAAG
TCCGCCATGCCCGAAGGCTACGTCCAGGAGCGCACCATCTTCTTCAAGGACGACGGCAACT
ACAAGACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACTACAAC
AGCCACAACGTCTATATCATGGCCGACAAGCAGAAGAACGGCATCAAGGTGAACTTCAAGA
TCCGCCACAACATCGAGGACGGCAGCGTGCAGCTCGCCGACCACTACCAGCAGAACACCCC
CATCGGCGACGGCCCCGTGCTGCTGCCCGACAACCACTACCTGAGCACCCAGTCCGCCCTGA
GCAAAGACCCCAACGAGAAGCGCGATCACATGGTCCTGCTGGAGTTCGTGACCGCCGCCGG
GATCACTCTCGGCATGGACGAGCTGTACAAGTAAAGGCGCGCCACCCCTGCAGGGAATTCC
CCCTGCAGGGAATTCGATATCAAGCTTATCGATAATCAACCTCTGGATTACAAAATTTGTGA
AAGATTGACTGGTATTCTTAACTATGTTGCTCCTTTTACGCTATGTGGATACGCTGCTTTAAT
GCCTTTGTATCATGCTATTGCTTCCCGTATGGCTTTCATTTTCTCCTCCTTGTATAAATCCTGG
TTGCTGTCTCTTTATGAGGAGTTGTGGCCCGTTGTCAGGCAACGTGGCGTGGTGTGCACTGTG
TTTGCTGACGCAACCCCCACTGGTTGGGGCATTGCCACCACCTGTCAGCTCCTTTCCGGGACT
TTCGCTTTCCCCCTCCCTATTGCCACGGCGGAACTCATCGCCGCCTGCCTTGCCCGCTGCTGG
ACAGGGGCTCGGCTGTTGGGCACTGACAATTCCGTGGTGTTGTCGGGGAAATCATCGTCCTT
TCCTTGGCTGCTCGCCTATGTTGCCACCTGGATTCTGCGCGGGACGTCCTTCTGCTACGTCCC
TTCGGCCCTCAATCCAGCGGACCTTCCTTCCCGCGGCCTGCTGCCGGCTCTGCGGCCTCTTCC
GCGTCTTCGCCTTCGCCCTCAGACGAGTCGGATCTCCCTTTGGGCCGCCTCCCCGCATCGATA
CCGAGCGCGCGATCGCAAACAAACCTCGAGAGATCTGTGATAGCGGCCATCAAGCTGGCCG
CGACTCTAGATCATAATCAGCCATACCACATTTGTAGAGGTTTTACTTGCTTTAAAAAACCTC
CCACACCTCCCCCTGAACCTGAAACATAAAATGAATGCAATTGTTGTTGTTAACTTGTTTATT
GCAGCTTATAATGGTTACAAATAAAGCAATAGCATCACAAATTTCACAAATAAAGCATTTTT
TTCACTGCATTCTAGTTGTGGTTTGTCCAAACTCATCAATGTATCAGCTTATCGATACCGCAT
GCACGTGCGGACCGAGCGGCCGCAGGAACCCCTAGTGATGGAGTTGGCCACTCCCTCTCTGCG
CGCTCGCTCGCTCACTGAGGCCGGGCGACCAAAGGTCGCCCGACGCCCGGGCTTTGCCCGGGC
GGCCTCAGTGAGCGAGCGAGCGCGCAGCTGCCTGCAGGGGCGCCTGATGCGGTATTTTCTCCT
TACGCATCTGTGCGGTATTTCACACCGCATACGTCAAAGCAACCATAGTACGCGCCCTGTAG
CGGCGCATTAAGCGCGGCGGGTGTGGTGGTTACGCGCAGCGTGACCGCTACACTTGCCAGC
GCCCTAGCGCCCGCTCCTTTCGCTTTCTTCCCTTCCTTTCTCGCCACGTTCGCCGGCTTTCCCC
GTCAAGCTCTAAATCGGGGGCTCCCTTTAGGGTTCCGATTTAGTGCTTTACGGCACCTCGACC
CCAAAAAACTTGATTTGGGTGATGGTTCACGTAGTGGGCCATCGCCCTGATAGACGGTTTTT
CGCCCTTTGACGTTGGAGTCCACGTTCTTTAATAGTGGACTCTTGTTCCAAACTGGAACAACA
CTCAACCCTATCTCGGGCTATTCTTTTGATTTATAAGGGATTTTGCCGATTTCGGCCTATTGG
TTAAAAAATGAGCTGATTTAACAAAAATTTAACGCGAATTTTAACAAAATATTAACGTTTAC
AATTTTATGGTGCACTCTCAGTACAATCTGCTCTGATGCCGCATAGTTAAGCCAGCCCCGAC
ACCCGCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGAAACG
CGCGAGACGAAAGGGCCTCGTGATACGCCTATTTTTATAGGTTAATGTCATGATAATAATGG
TTTCTTAGACGTCAGGTGGCACTTTTCGGGGAAATGTGCGCGGAACCCCTATTTGTTTATTTT
TCTAAATACATTCAAATATGTATCCGCTCATGAGACAATAACCCTGATAAATGCTTCAATAA
TATTGAAAAAGGAAGAGTATGAGTATTCAACATTTCCGTGTCGCCCTTATTCCCTTTTTTGCG
GCATTTTGCCTTCCTGTTTTTGCTCACCCAGAAACGCTGGTGAAAGTAAAAGATGCTGAAGA
TCAGTTGGGTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATGTGGCGCGG
TATTATCCCGTATTGACGCCGGGCAAGAGCAACTCGGTCGCCGCATACACTATTCTCAGAAT
GACTTGGTTGAGTACTCACCAGTCACAGAAAAGCATCTTACGGATGGCATGACAGTAAGAG
AATTATGCAGTGCTGCCATAACCATGAGTGATAACACTGCGGCCAACTTACTTCTGACAACG
ATCGGAGGACCGAAGGAGCTAACCGCTTTTTTGCACAACATGGGGGATCATGTAACTCGCCT
TGATCGTTGGGAACCGGAGCTGAATGAAGCCATACCAAACGACGAGCGTGACACCACGATG
CCTGTAGCAATGGCAACAACGTTGCGCAAACTATTAACTGGCG
SEQ ID NO: 14-GRE 12 portion
CTTTAGAGGGGGAAACTGCCTTTTGAGTTGTTTATATATAAAGTTATTTAAATAATGAAGAT
CATTTTTTTCTGCCTATAATGTTTTTCTTGAGATGATGCTTTCTTGAAAAAAATATTTTCAAAG
GCTGAAAACAAATACATAAGAACTCAGTAAACTCGGGAAGTGTTTAGCTTCATAATCAGACT
GTGCAGAAGATAGGAAGCAGCAGCCGGATCCACAGCCTCTGATTGTCCCAAATCACAGGAG
TCATCA
SEQ ID NO: 15-GRE 22 portion
AGCCAGGACTACACAGAGAAACCCTGTCTCAAAAAAACAAAATCAAAACAAAACAAACAA
ACAAAAAAGCTAATGACTCCATCATGACTGTAACAAACACATCAGTGCGGCAGTGAGAGCC
CGTCTGTCAGCATCAGCAACAGCATTAGTCAGACTGTATTTGTGAGCATATTTGCTTAGGTCT
CTTCTAAATACCCTTCACTTTTCTCTCAGAGAAACCCAGTTCATCGTATTCTGAAAAGGAGCG
GCCGTAAA
SEQ ID NO: 16-GRE 44 portion
GTTCAGTACCCAGACACTCCCATACCCTTATTTAGAAGAAATAAATATCATCAAGTCATAAT
ATCCTTGACTGATTAAGAAAGCCACTTTGTAAGTGTTTATTAAACTGTCAAGAAACTTACAG
AATTTACTACATGATCGTTAGAATAACTTTGAGTCAGGACATATTTGATATGACTTAATCATA
CTCCCTCCAAAAGGAAATAAGGCTTTGTGAAGGTAAATTATTTCTTCCTGGGTTGGATATGT
GTTTAT
SEQ ID NO: 17-GRE 12
CTTTAGAGGGGGAAACTGCCttttgagttgtttatatataaagttatttaaataatgaagatcatttttttctgcctataatgtttttcttgagatga
tgctttcttgaaaaaaatacaaaggctgaaaacaaatacataagaactcagtaaactcgggaagtgtttagcttcataatcagactgtgcagaagatag
gaagcagcagccggatccacagcctctgattgtcccaaatcacaggagtcatcaactgagtactccaaaaaggaaaacaagccacagctaaaagat
atgagcataatgtgtaccataatctcacagtggctgttttagaaccaagagtgtttgtgacttaatttgaatttctcaatgcaacatttctcaaaaattccttaaac
gtcatgtcatagatgatttattatgtacaaaacataactgttgagaaactccatttccttgccttctgggaggaaccttaggaaacatcagcagcaggtgcaaa
gtattccatagagagagggctggcataaagaacatatttattcatcagttccaaatttccctgcttctgagggcttaaaaagagggatttcttgagctgagga
aattaaaaacaaaacaaacaactatgctgaaagaggactagaaatgttctgggatattgtgaaatctagacttgaaattccttctcatttccttatgcacagatt
ttaacaCCCTTGGTTTCTTCGGAGTA
SEQ ID NO: 18-GRE 22
AGCCAGGACTACACAGAGAAaccctgtctcaaaaaaacaaaatcaaaacaaaacaaacaaacaaaaaagctaatgactccatcatga
ctgtaacaaacacatcagtgcggcagtgagagcccgtctgtcagcatcagcaacagcattagtcagactgtatttgtgagcatatttgcttaggtctcttcta
aatacccttcacttttctctcagagaaacccagttcatcgtattctgaaaaggagcggccgtaaaggactgatcctgtctgaagcactttggtataaaagttgc
ttagcagtggggcagaaaagaaaaaaagcaattaagtttatatttagtgatctatctatacacatctggagcacatttgggaaagaattcaaaagggccaatt
cattgcatgcctcctgctacagaacgagtgtgggagtcaagctgcgatttccacagcatcagacatttattgttgacttcaaaaagttctcccacttatgtgta
attactatcctagcaaatggctctgaaatttcagcttcttaagcataaggcagagtggtcctttaaaagtaaaataaaacgtaggccctatgagataaaattaa
gataaattaagaatcagttacttccaagacgaagcacttatggtgcatgccttcttatataaagcagatccttaccatgtatgtgtgctgtttgcTTGCCA
AGACCAAGATGTCT
SEQ ID NO: 19-GRE 44
GTTCAGTACCCAGACACTCCcatacccttatttagaagaaataaatatcatcaagtcataatatccttgactgattaagaaagccactttgt
aagtgtttattaaactgtcaagaaacttacagaatttactacatgatcgttagaataactagagtcaggacatatttgatatgacttaatcatactccctccaaaa
ggaaataaggctagtgaaggtaaattatacttcctgggttggatatgtgtttatggagtgatcattcagctgttcccaaccttcattctgaaaaggcctcagaa
cacttcatgatgaatcaagctgtatcctgaatagagtaaaatgaaccacttcgtaggaactatggtgtcaccacatcagcaattcttattgaaaagtgtgcattt
cttattcacatatttcaaagatggtattccagaggagtgattttctcaatgtatttttcatctacaagccttcattttaagcctaccaccgtgtgtgttttcaagacag
caattatcgttttaaaatgtgcaggtctagcttgagcttctcagcaagtttctatgccaaagaaaacaccaatcctttccatttactgagaatcaatgtttaatcct
cctttttgttctcatacttattacaaatcataaagaattctgagtgtcagtagataactagaagctccatgtACCATTCCTGCTCCTTATTGA
SEQ ID NO: 20-GRE 19
GCAGAATCAGATAAGCAGAATGAatcttcattataatgtactcatatccaacagtttactgactttctgatctgagtatgaatctgagtct
atttcctaaccctactaatagtcaatattattatttatttctatgtctacactggcaggcaccatttacaacccggtcatcctgtagcatcattctatgtattta
catatttcctggtcctcctgggacaatattctagcatagttccccaccttccttcctcagcccagctgcagactcctctcttctttctttctcagtatgattgaatac
atttaaaatcaacatcatctcttccactcgcattcctctcccatctgctgatgcccacccaatccttcttttggaatcagatttaatatgaatctttaaaatcaaaat
catcttctgtttctttgccctaatccagcagctgcagatttcttctcccggcactgttctccgtctgcagctcaccaaaatgcttcttagaaaaatatgcagttgtt
tttctcccctatccaaaaggctggaactttcctggcttcccaattatacaatttatgcttcttttaaggattgtgaagatgatattattagaagttgagcgaattgg
ggctgtgtatggaaggaagggaagtactttaagtgaatgatattgggtatAAAATGGCACATAGGGCTCT
SEQ ID NO: 21-GRE 80
AGTAGAGGCCACAGCTAAGAagtgmctctatctgcaggtgcaaagggagcgtggataaatgatttttgtaaatctacctcaatgctgta
cttcaagtatttca cacacaatccattaagagatgaaatggaatcagtaggtcattacggtcagaagtatttaaatgatttaatatgactggagatataaat
ctatactgtagtccttgatacttctattcatccgaaaacctttattattcaaaagtgctcaccaggttctgcctcatgcagaaaaataccctcaagcagaggact
gttgcatattcttaccatattctcccaaacttgaatggtaagcagttgtgcatcagtaccaccacccgctgccacgggtgtgcatatggagtctcacaaataa
gaacataaaataatgacaggaagaaaacaaaccaaaagctaaaattaccagtggagctgattagcatatgtataagagacacttgtacagatgtgggttg
ctttctttagaacctaagttctcagagcagtgattcttcatcattttttgagttgtgaagtcttattatttgtttgctttttatgatcatcaccagctcctcccaaaagca
tatttttzaatgggaagaaataattttatttttgaacatttgctcttatttttaccctcccaaagagggtaaaaaacgctctagaggtagcctagttatcattAAT
TCGGAATCAGCAGCCTC
SEQ ID NO: 22-rAAV-GRE12-Gq-DREADD-tdTomato
aactacttactctagcttcccggcaacaattaatagactggatggaggcggataaagttgcaggaccacttctgcgctcggcccttccggctggctggttta
ttgctgataaatctggagccggtgagcgtgggtctcgcggtatcattgcagcactggggccagatggtaagccctcccgtatcgtagttatctacacgacg
gggagtcaggcaactatggatgaacgaaatagacagatcgctgagataggtgcctcactgattaagcattggtaactgtcagaccaagtttactcatatata
ctttagattgatttaaaacttcatttttaatttaaaaggatctaggtgaagatcctttttgataatctcatgaccaaaatcccttaacgtgagttttcgttccactgag
cgtcagaccccgtagaaaagatcaaaggatcttcttgagatcctttttttctgcgcgtaatctgctgcttgcaaacaaaaaaaccaccgctaccagcggtgg
tttgtttgccggatcaagagctaccaactctttttccgaaggtaactggcttcagcagagcgcagataccaaatactgtccttctagtgtagccgtagttaggc
caccacttcaagaactctgtagcaccgcctacatacctcgctctgctaatcctgttaccagtggctgctgccagtggcgataagtcgtgtcttaccgggttg
gactcaagacgatagttaccggataaggcgcagcggtcgggctgaacggggggttcgtgcacacagcccagcttggagcgaacgacctacaccgaa
ctgagatacctacagcgtgagctatgagaaagcgccacgcttcccgaagggagaaaggcggacaggtatccggtaagcggcagggtcggaacagga
gagcgcacgagggagcttccagggggaaacgcctggtatctttatagtcctgtcgggtttcgccacctctgacttgagcgtcgatttttgtgatgctcgtca
ggggggcggagcctatggaaaaacgccagcaacgcggcctttttacggttcctggccttttgctggccttttgctcacatgtcctgcaggcagctgcgcg
ctcgctcgctcactgaggccgcccgggcaaagcccgggcgtcgggcgacctaggtcgcccggcctcagtgagcgagcgagcgcgcagagaggga
gtggccaactccatcactaggggttcctgcggccgcacgcgtttaatCTTTAGAGGGGGAAACTGCCttttgagttgtttatatataaagtt
atttaaataatgaagatcatttttttctgcctataatgtttttcttgagatgatgctttcttgaaaaaaatattttcaaaggctgaaaacaaatacataagaactcagt
aaactcgggaagtgtttagcttcataatcagactgtgcagaagataggaagcagcagccggatccacagcctctgattgtcccaaatcacaggagtcatc
aactgagtactccaaaaaggaaaacaagccattttcagctaaaagatatgagcataatgtgtaccataatctcacagtggctgttttagaaccaagagtgttt
gtgacttaatttgaatttctcaatgcaacatttctcaaaaattccttaaacgtcatgtcatagatgatttattatgtacaaaacataactgttgagaaactccatttc
cttgccttctgggaggaaccttaggaaacatcagcagcaggtgcaaagtattccatagagagagggctggcataaagaacatatttattcatcagttccaa
atttccctgcttctgagggcttaaaaagagggatttcttgagctgaggaaattaaaaacaaaacaaacaactatgctgaaagaggactagaaatgttctggg
atattgtgaaatctagacttgaaattccttctcataccttatgcacagattttaacaCCCTTGGTTTCTTCGGAGTAGTCGACtgatatc
gaattcctgcagcccgggctgggcataaaagtcagggcagagccatctattgcttacatttgcttctagcctgcaggtcgaggagcgcagccttccagaa
gcagagcgcggcgccttaagctgcagaagttggtcgtgaggcactgggcaggtaagtatcaaggttacaagacaggtttaaggagaccaatagaaact
gggcttgtcgagacagagaagactcttgcgtactgataggcacctattggtcttactgacatccactttgcctttctctccacaggtgtccactcccaGTgc
caccatgaccttgcacaataacagtacaacctcgcctttgtttccaaacatcagctcctcctggatacacagcccctccgatgcagggctgcccccgggaa
ccgtcactcatttcggcagctacaatgtttctcgagcagctggcaatttctcctctccagacggtaccaccgatgaccctctgggaggtcataccgtctggc
aagtggtcttcatcgctacttaacgggcatcctggccttggtgaccatcatcggcaacatcctggtaattgtgtcatttaaggtcaacaagcagctgaagac
ggtcaacaactacttcctcttaagcctggcctgtgccgatctgattatcggggtcatttcaatgaatctgtttacgacctacatcatcatgaatcgatgggcctt
agggaacttggcctgtgacctctggcttgccattgactgcgtagccagcaatgcctctgttatgaatcttctggtcatcagctttgacagatacttttccatcac
gaggccgctcacgtaccgagccaaacgaacaacaaagagagccggtgtgatgatcggtctggcttgggtcatctcctttgtcctttgggctcctgccatct
tgttctggcaatactttgttggaaagagaactgtgcctccgggagagtgcttcattcagttcctcagtgagcccaccattacttttggcacagccatcgctggt
ttttatatgcctgtcaccattatgactattttatactggaggatctataaggaaactgaaaagcgtaccaaagagcttgctggcctgcaagcctctgggacag
aggcagagacagaaaactttgtccaccccacgggcagttctcgaagctgcagcagttacgaacttcaacagcaaagcatgaaacgctccaacaggagg
aagtatggccgctgccacttctggttcacaaccaagagctggaaacccagctccgagcagatggaccaagaccacagcagcagtgacagttggaaca
acaatgatgctgctgcctccctggagaactccgcctcctccgacgaggaggacattggctccgagacgagagccatctactccatcgtgctcaagcttcc
gggtcacagcaccatcctcaactccaccaagttaccctcatcggacaacctgcaggtgcctgaggaggagctggggatggtggacttggagaggaaa
gccgacaagctgcaggcccagaagagcgtggacgatggaggcagttttccaaaaagcttctccaagcttcccatccagctagagtcagccgtggacac
agctaagacttctgacgtcaactcctcagtgggtaagagcacggccactctacctctgtccttcaaggaagccactctggccaagaggtttgctctgaaga
ccagaagtcagatcactaagcggaaaaggatgtccctggtcaaggagaagaaagcggcccagaccctcagtgcgatcttgcttgccttcatcatcacttg
gaccccatacaacatcatggttctggtgaacaccttttgtgacagctgcatacccaaaaccttttggaatctgggctactggctgtgctacatcaacagcacc
gtgaaccccgtgtgctatgctctgtgcaacaaaacattcagaaccactttcaagatgctgctgctgtgccagtgtgacaaaaaaaagaggcgcaagcagc
agtaccagcagagacagtcggtcatttttcacaagcgcgcacccgagcaggccttgaaggatcccccggtcgccaccatggtgagcaagggcgagga
ggataacatggccatcatcaaggagttcatgcgcttcaaggtgcacatggagggctccgtgaacggccacgagttcgagatcgagggcgagggcgag
ggccgcccctacgagggcacccagaccgccaagctgaaggtgaccaagggtggccccctgcccttcgcctgggacatcctgtcccctcagttcatgta
cggctccaaggcctacgtgaagcaccccgccgacatccccgactacttgaagctgtccttccccgagggcttcaagtgggagcgcgtgatgaacttcga
ggacggcggcgtggtgaccgtgacccaggactcctccctgcaggacggcgagttcatctacaaggtgaagctgcgcggcaccaacttcccctccgac
ggccccgtaatgcagaagaagaccatgggctgggaggcctcctccgagcggatgtaccccgaggacggcgccctgaagggcgagatcaagcagag
gctgaagctgaaggacggcggccactacgacgctgaggtcaagaccacctacaaggccaagaagcccgtgcagctgcccggcgcctacaacgtcaa
catcaagttggacatcacctcccacaacgaggactacaccatcgtggaacagtacgaacgcgccgagggccgccactccaccggcggcatggacga
gctgtacaagtaagaattccccctgcagggaattcgatatcaagcttatcgataatcaacctctggattacaaaatttgtgaaagattgactggtattcttaact
atgttgctccttttacgctatgtggatacgctgctttaatgcctttgtatcatgctattgcttcccgtatggctttcattttctcctccttgtataaatcctggttgctgt
ctctttatgaggagttgtggcccgttgtcaggcaacgtggcgtggtgtgcactgtgtttgctgacgcaacccccactggttggggcattgccaccacctgtc
agctcctttccgggactttcgctttccccctccctattgccacggcggaactcatcgccgcctgccttgcccgctgctggacaggggctcggctgttgggc
actgacaattccgtggtgttgtcggggaaatcatcgtcctttccttggctgctcgcctatgttgccacctggattctgcgcgggacgtccttctgctacgtccc
ttcggccctcaatccagcggaccttccttcccgcggcctgctgccggctctgcggcctcttccgcgtcttcgccttcgccctcagacgagtcggatctccct
ttgggccgcctccccgcatcgataccgagcgcGCGATcgcAAACAAACCtcgagagatctgtgatagcggccatcaagctggccgcgac
tctagatcataatcagccataccacatttgtagaggttttacttgctttaaaaaacctcccacacctccccctgaacctgaaacataaaatgaatgcaattgttg
ttgttaacttgtttattgcagcttataatggttacaaataaagcaatagcatcacaaatttcacaaataaagcatttttttcactgcattctagttgtggtttgtccaa
actcatcaatgtatcagcttatcgataccgcatgcacgtgcggaccgagcggccgcaggaacccctagtgatggagttggccactccctctctgcgcgct
cgctcgctcactgaggccgggcgaccaaaggtcgcccgacgcccgggctttgcccgggcggcctcagtgagcgagcgagcgcgcagctgcctgca
ggggcgcctgatgcggtattttctccttacgcatctgtgcggtatttcacaccgcatacgtcaaagcaaccatagtacgcgccctgtagcggcgcattaag
cgcggcgggtgtggtggttacgcgcagcgtgaccgctacacttgccagcgccctagcgcccgctcctttcgctttcttcccttcctttctcgccacgttcgc
cggctttccccgtcaagctctaaatcgggggctccctttagggttccgatttagtgctttacggcacctcgaccccaaaaaacttgatttgggtgatggttca
cgtagtgggccatcgccctgatagacggtttttcgccctttgacgttggagtccacgttctttaatagtggactcttgttccaaactggaacaacactcaaccc
tatctcgggctattcttttgatttataagggattttgccgatttcggcctattggttaaaaaatgagctgatttaacaaaaatttaacgcgaattttaacaaaatatt
aacgtttacaattttatggtgcactctcagtacaatctgctctgatgccgcatagttaagccagccccgacacccgccaacacccgctgacgcgccctgac
gggcttgtctgctcccggcatccgcttacagacaagctgtgaccgtctccgggagctgcatgtgtcagaggcaccgtcatcaccgaaacgcgcgag
acgaaagggcctcgtgatacgcctatttttataggttaatgtcatgataataatggtttcttagacgtcaggtggcacttttcggggaaatgtgcgcggaacc
cctatttgtttatttttctaaatacattcaaatatgtatccgctcatgagacaataaccctgataaatgcttcaataatattgaaaaaggaagagtatgagtattca
acatttccgtgtcgcccttattcccttttttgcggcattttgccttcctgtttttgctcacccagaaacgctggtgaaagtaaaagatgctgaagatcagttgggt
gcacgagtgggttacatcgaactggatctcaacagcggtaagatccttgagagttttcgccccgaagaacgttttccaatgatgagcacttttaaagttctgc
tatgtggcgcggtattatcccgtattgacgccgggcaagagcaactcggtcgccgcatacactattctcagaatgacttggttgagtactcaccagtcaca
gaaaagcatcttacggatggcatgacagtaagagaattatgcagtgctgccataaccatgagtgataacactgcggccaacttacttctgacaacgatcgg
aggaccgaaggagctaaccgcttttttgcacaacatgggggatcatgtaactcgccttgatcgttgggaaccggagctgaatgaagccataccaaacga
cgagcgtgacaccacgatgcctgtagcaatggcaacaacgttgcgcaaactattaactggcg

See, for example, Supplementary file 1 of Hrvatin et al supra, for list of 323,369 genomic coordinates used to enrich for GREs that could be useful reagents to study and manipulate interneurons across mammalian species, including humans, representing a union of cortical neuron ATAC-seq-accessible regions identified across dozens of experiments. In some embodiments of any of the aspects, the genomic coordinates refer to the genome of C57BL/6J mice (Mus musculus; e.g., GRCm38/mm10, December 2011).

Table 3 below is a list of the top 287 most enriched GREs, which were selected for functional screening to identify enhancers that drive gene expression selectively in SST interneurons of the primary visual cortex. In some embodiments of any of the aspects, the genomic coordinates refer to the genome of C57BL/6J mice (Mus musculus; e.g., GRCm38/mm10, December 2011).

TABLE 3
Genomic locations of GRE1-GRE287 (TSS indicates transcriptional start site).

| | | | | | Distance | Nearest |
|---|---|---|---|---|---|---|
| | Chr | Start | End | Annotation | to TSS | PromoterID |
| master_174202 | chr2 | 1.17E+08 | 1.17E+08 | intron | 1292 | NM_026104 |
| | | | | (NM_026104, | | |
| | | | | intron 2 of 11) | | |
| master_101787 | chr14 | 87063811 | 87064111 | intron | 77153 | NM_019670 |
| | | | | (NM_019670, | | |
| | | | | intron 4 of 27) | | |
| master_100995 | chr14 | 80144647 | 80144947 | Intergenic | 144495 | NM_001030294 |
| master_10184 | chr1 | 81945313 | 81945613 | Intergenic | −186453 | NR_105762 |
| master_194536 | chr3 | 86507540 | 86507874 | intron | 40576 | NM_011839 |
| | | | | (NM_001077687, | | |
| | | | | intron 37 of 50) | | |
| master_206695 | chr4 | 13247108 | 13247408 | Intergenic | 340421 | NM_001171801 |
| master_85259 | chr13 | 75674530 | 75674830 | Intergenic | 29634 | NR_030451 |
| master_211290 | chr4 | 51486496 | 51486796 | Intergenic | 269968 | NM_001162865 |
| master_142964 | chr18 | 17976519 | 17976819 | Intergenic | −395679 | NR_045374 |
| master_63024 | chr12 | 25235134 | 25235542 | Intergenic | 106164 | NR_045844 |
| master_137958 | chr17 | 69736636 | 69736936 | Intergenic | 297460 | NM_001145192 |
| master_111792 | chr15 | 52005639 | 52005939 | Intergenic | −14029 | NM_009009 |
| master_101297 | chr14 | 82682165 | 82682465 | Intergenic | −1761248 | NM_001013753 |
| master_101845 | chr14 | 87374272 | 87374572 | Intergenic | −42161 | NM_172605 |
| master_232280 | chr5 | 54271036 | 54271407 | Intergenic | −78771 | NR_038045 |
| master_102497 | chr14 | 94023229 | 94023529 | Intergenic | −132710 | NM_001271798 |
| master_67193 | chr12 | 58554757 | 58555057 | Intergenic | −285649 | NM_025809 |
| master_300352 | chr9 | 33901745 | 33902045 | Intergenic | −140476 | NR_040744 |
| master_165379 | chr2 | 45924258 | 45924558 | Intergenic | 227803 | NR_040497 |
| master_226748 | chr5 | 13006966 | 13007266 | Intergenic | −389668 | NM_001243072 |
| master_168633 | chr2 | 72677275 | 72677713 | Intergenic | −26016 | NR_040503 |
| master_168094 | chr2 | 69084461 | 69084761 | intron | −51189 | NM_181547 |
| | | | | (NM_172856, | | |
| | | | | intron 8 of 9) | | |
| master_250505 | chr6 | 41759626 | 41759926 | Intergenic | 12521 | NM_146576 |
| master_169135 | chr2 | 76686648 | 76687525 | intron | 11771 | NM_031256 |
| | | | | (NM_031256, | | |
| | | | | intron 4 of 7) | | |
| master_213877 | chr4 | 71433118 | 71433418 | Intergenic | 767625 | NM_011599 |
| master_48723 | chr11 | 44977679 | 44978307 | intron | 138935 | NR_045100 |
| | | | | (NM_001290709, | | |
| | | | | intron 11 of 15) | | |
| master_125071 | chr16 | 47293191 | 47293491 | Intergenic | −796374 | NM_021497 |
| master_284632 | chr8 | 41858270 | 41858585 | intron | 31160 | NR_045497 |
| | | | | (NR_045497, | | |
| | | | | intron 1 of 5) | | |
| master_126619 | chr16 | 64000736 | 64001131 | Intergenic | −136776 | NM_010140 |
| master_13877 | chr1 | 1.08E+08 | 1.08E+08 | Intergenic | −101775 | NR_102306 |
| master_149404 | chr18 | 65322278 | 65322578 | intron | 71460 | NM_001037294 |
| | | | | (NM_001037294, | | |
| | | | | intron 4 of 12) | | |
| master_17921 | chr1 | 1.37E+08 | 1.37E+08 | Intergenic | −492293 | NR_029795 |
| master_273562 | chr7 | 94827754 | 94828054 | Intergenic | −1382733 | NM_011858 |
| master_1929 | chr1 | 18148248 | 18148560 | Intergenic | −11353 | NM_030033 |
| master_209025 | chr4 | 33467640 | 33468055 | intron | 29766 | NR_106198 |
| | | | | (NM_011884, | | |
| | | | | intron 14 of 15) | | |
| master_48719 | chr11 | 44943813 | 44944113 | intron | 172965 | NR_045100 |
| | | | | (NM_001290709, | | |
| | | | | intron 10 of 15) | | |
| master_231206 | chr5 | 44729846 | 44730146 | intron | 69750 | NM_010698 |
| | | | | (NM_001286348, | | |
| | | | | intron 1 of 8) | | |
| master_206581 | chr4 | 12317952 | 12318252 | Intergenic | −146087 | NM_026558 |
| master_145539 | chr18 | 37732557 | 37732857 | exon | 1553 | NM_033577 |
| | | | | (NM_033577, | | |
| | | | | exon 1 of 4) | | |
| master_234138 | chr5 | 70659320 | 70659620 | Intergenic | 183147 | NM_010252 |
| master_29489 | chr10 | 28555586 | 28555886 | intron | −112659 | NM_178666 |
| | | | | (NM_008983, | | |
| | | | | intron 14 of 31) | | |
| master_32108 | chr10 | 49877496 | 49877796 | Intergenic | −88892 | NM_001111268 |
| master_213912 | chr4 | 71672154 | 71672454 | Intergenic | 528589 | NM_011599 |
| master_203978 | chr3 | 1.51E+08 | 1.51E+08 | Intergenic | −645477 | NM_133222 |
| master_303924 | chr9 | 59125842 | 59126142 | Intergenic | −89551 | NM_001042752 |
| master_24617 | chr1 | 1.88E+08 | 1.88E+08 | intron | −190934 | NM_011935 |
| | | | | (NM_001243792, | | |
| | | | | intron 2 of 7) | | |
| master_284637 | chr8 | 41869988 | 41870288 | intron | 42871 | NR_045497 |
| | | | | (NR_045497, | | |
| | | | | intron 1 of 5) | | |
| master_27246 | chr10 | 13170785 | 13171640 | intron | 6811 | NM_029172 |
| | | | | (NM_029172, | | |
| | | | | intron 2 of 5) | | |
| master_214332 | chr4 | 76932421 | 76932721 | intron | 1279324 | NM_011211 |
| | | | | (NM_011211, | | |
| | | | | intron 8 of 39) | | |
| master_107992 | chr15 | 16637595 | 16637895 | Intergenic | −140356 | NM_009869 |
| master_47913 | chr11 | 36878046 | 36878346 | intron | 66045 | NM_001290702 |
| | | | | (NM_001290702, | | |
| | | | | intron 1 of 27) | | |
| master_201188 | chr3 | 1.32E+08 | 1.32E+08 | intron | 86516 | NM_020265 |
| | | | | (NM_020265, | | |
| | | | | intron 1 of 3) | | |
| master_108055 | chr15 | 17614957 | 17615325 | Intergenic | 754486 | NR_045711 |
| master_207201 | chr4 | 17636014 | 17636526 | Intergenic | −217212 | NM_019724 |
| master_107942 | chr15 | 16056105 | 16056405 | Intergenic | −721846 | NM_009869 |
| master_277752 | chr7 | 1.3E+08 | 1.3E+08 | Intergenic | −137134 | NR_046077 |
| master_5879 | chr1 | 48524047 | 48524347 | Intergenic | −1057072 | NR_105768 |
| master_211266 | chr4 | 51127803 | 51128117 | Intergenic | −88718 | NM_001162865 |
| master_15237 | chr1 | 1.19E+08 | 1.19E+08 | Intergenic | 168177 | NM_023755 |
| master_127446 | chr16 | 75843639 | 75844145 | Intergenic | 65374 | NM_023380 |
| master_207259 | chr4 | 18410432 | 18410732 | Intergenic | 557100 | NM_019724 |
| master_85915 | chr13 | 81182137 | 81182437 | intron | 218213 | NR_015587 |
| | | | | (NM_054053, | | |
| | | | | intron 86 of 89) | | |
| master_185216 | chr3 | 16204031 | 16204750 | exon | 21207 | NM_001145919 |
| | | | | (NM_001145919, | | |
| | | | | exon 4 of 5) | | |
| master_286157 | chr8 | 54451716 | 54452016 | Intergenic | 78132 | NM_029701 |
| master_211163 | chr4 | 50006897 | 50007259 | Intergenic | −161309 | NM_001276355 |
| master_247854 | chr6 | 22097748 | 22098067 | intron | 111997 | NM_001081351 |
| | | | | (NM_001081351, | | |
| | | | | intron 6 of 21) | | |
| master_67398 | chr12 | 60382709 | 60383009 | Intergenic | 1138961 | NR_045049 |
| master_175066 | chr2 | 1.23E+08 | 1.23E+08 | Intergenic | 409539 | NM_021507 |
| master_316282 | chrX | 61637693 | 61638003 | Intergenic | −71768 | NM_001018087 |
| master_39511 | chr10 | 1.03E+08 | 1.03E+08 | Intergenic | 82244 | NM_146240 |
| master_188695 | chr3 | 42418127 | 42418427 | Intergenic | 675659 | NM_027271 |
| master_166708 | chr2 | 57897305 | 57897605 | Intergenic | −100700 | NM_172855 |
| master_297161 | chr9 | 5752703 | 5753057 | Intergenic | 407404 | NM_009808 |
| master_55384 | chr11 | 90807428 | 90807728 | intron | 2887 | NR_045956 |
| | | | | (NR_045956, | | |
| | | | | intron 1 of 2) | | |
| master_284777 | chr8 | 43377629 | 43378144 | Intergenic | −70877 | NM_009556 |
| master_271292 | chr7 | 71374000 | 71374404 | Intergenic | 932393 | NM_001024703 |
| master_40675 | chr10 | 1.13E+08 | 1.13E+08 | intron | 27080 | NR_040579 |
| | | | | (NR_040579, | | |
| | | | | intron 2 of 6) | | |
| master_258714 | chr6 | 1.04E+08 | 1.04E+08 | Intergenic | 433631 | NM_007697 |
| master_154372 | chr19 | 14681284 | 14681836 | Intergenic | −83577 | NM_011600 |
| master_216325 | chr4 | 94392090 | 94392390 | Intergenic | 164556 | NM_026368 |
| master_104183 | chr14 | 1.06E+08 | 1.06E+08 | Intergenic | 153711 | NR_073203 |
| master_154310 | chr19 | 13749599 | 13749899 | exon | 641 | NM_146990 |
| | | | | (NM_146990, | | |
| | | | | exon 1 of 1) | | |
| master_246461 | chr6 | 9842484 | 9842784 | Intergenic | 892615 | NM_008751 |
| master_180377 | chr2 | 1.62E+08 | 1.62E+08 | exon | −255497 | NR_040617 |
| | | | | (NM_001291151, | | |
| | | | | exon 7 of 31) | | |
| master_17924 | chr1 | 1.38E+08 | 1.38E+08 | Intergenic | −458836 | NR_029795 |
| master_216328 | chr4 | 94407557 | 94408022 | Intergenic | 149007 | NM_026368 |
| master_127878 | chr16 | 79870672 | 79870972 | Intergenic | −779725 | NM_178855 |
| master_31540 | chr10 | 44444438 | 44444760 | intron | 14088 | NM_007548 |
| | | | | (NM_007548, | | |
| | | | | intron 4 of 6) | | |
| master_90969 | chr13 | 1.17E+08 | 1.17E+08 | exon | 46859 | NM_010330 |
| | | | | (NM_010330, | | |
| | | | | exon 6 of 9) | | |
| master_152861 | chr18 | 87754010 | 87754458 | Intergenic | 1041186 | NM_172633 |
| master_83032 | chr13 | 57459655 | 57460086 | intron | 448462 | NM_009262 |
| | | | | (NM_001166464, | | |
| | | | | intron 7 of 10) | | |
| master_315069 | chrX | 42393500 | 42393800 | Intergenic | −108958 | NM_011364 |
| master_177079 | chr2 | 1.38E+08 | 1.38E+08 | Intergenic | −156607 | NM_001025431 |
| master_98792 | chr14 | 64822875 | 64823265 | 3′UTR | 126777 | NM_177338 |
| | | | | (NM_177338, | | |
| | | | | exon 10 of 10) | | |
| master_7522 | chr1 | 61539768 | 61540394 | Intergenic | −98743 | NM_001081050 |
| master_234051 | chr5 | 69379142 | 69379455 | Intergenic | −37589 | NM_175519 |
| master_253151 | chr6 | 63180265 | 63180680 | Intergenic | −76385 | NM_008167 |
| master_3497 | chr1 | 33032816 | 33033116 | Intergenic | −485673 | NM_001164286 |
| master_67191 | chr12 | 58551174 | 58551474 | Intergenic | −282066 | NM_025809 |
| master_307584 | chr9 | 88135961 | 88136392 | Intergenic | −191433 | NM_011851 |
| master_21944 | chr1 | 1.69E+08 | 1.69E+08 | Intergenic | 123264 | NM_023284 |
| master_18469 | chr1 | 1.43E+08 | 1.43E+08 | Intergenic | −656000 | NM_020025 |
| master_292268 | chr8 | 99205235 | 99205535 | intron | 193498 | NR_110579 |
| | | | | (NM_007667, | | |
| | | | | intron 4 of 11) | | |
| master_258969 | chr6 | 1.07E+08 | 1.07E+08 | Intergenic | −247071 | NM_175357 |
| master_278030 | chr7 | 1.32E+08 | 1.32E+08 | Intergenic | −3940 | NM_018867 |
| master_292154 | chr8 | 97733249 | 97733712 | Intergenic | −1187507 | NR_039538 |
| master_94135 | chr14 | 27544028 | 27544385 | Intergenic | −35746 | NM_177111 |
| master_202401 | chr3 | 1.4E+08 | 1.4E+08 | Intergenic | 386637 | NR_105798 |
| master_296959 | chr8 | 1.29E+08 | 1.29E+08 | exon | 143503 | NR_035436 |
| | | | | (NM_008737, | | |
| | | | | exon 17 of 17) | | |
| master_242798 | chr5 | 1.33E+08 | 1.33E+08 | Intergenic | −676679 | NM_177047 |
| master_100196 | chr14 | 75397464 | 75397764 | Intergenic | −9468 | NR_030568 |
| master_5836 | chr1 | 48109335 | 48109677 | Intergenic | −642381 | NR_105768 |
| master_283415 | chr8 | 30998708 | 30999077 | Intergenic | −90770 | NM_025869 |
| master_176631 | chr2 | 1.34E+08 | 1.34E+08 | Intergenic | 68147 | NM_010403 |
| master_85927 | chr13 | 81283259 | 81283559 | exon | 319335 | NR_015587 |
| | | | | (NM_054053, | | |
| | | | | exon 84 of 90) | | |
| master_24377 | chr1 | 1.86E+08 | 1.86E+08 | Intergenic | −230408 | NM_146106 |
| master_246625 | chr6 | 11306040 | 11306386 | Intergenic | −331835 | NR_033776 |
| master_191318 | chr3 | 62048084 | 62048384 | Intergenic | −290543 | NM_001081295 |
| master_301220 | chr9 | 41429732 | 41430032 | intron | 53321 | NR_040725 |
| | | | | (NR_040725, | | |
| | | | | intron 4 of 4) | | |
| master_202524 | chr3 | 1.42E+08 | 1.42E+08 | exon | 74170 | NM_001277218 |
| | | | | (NM_001277218, | | |
| | | | | exon 7 of 11) | | |
| master_215096 | chr4 | 84286675 | 84286975 | intron | 388261 | NM_172870 |
| | | | | (NM_172870, | | |
| | | | | intron 5 of 5) | | |
| master_307975 | chr9 | 91413996 | 91414296 | Intergenic | 45174 | NM_009576 |
| master_293057 | chr8 | 1.06E+08 | 1.06E+08 | exon | 21800 | NM_145824 |
| | | | | (NM_145824, | | |
| | | | | exon 3 of 14) | | |
| master_289251 | chr8 | 78643062 | 78643858 | intron | 134132 | NM_001282108 |
| | | | | (NM_029736, | | |
| | | | | intron 5 of 11) | | |
| master_156217 | chr19 | 31180654 | 31180998 | intron | 35570 | NR_002849 |
| | | | | (NM_001013833, | | |
| | | | | intron 3 of 17) | | |
| master_122150 | chr16 | 24053277 | 24053830 | Intergenic | −64941 | NM_009744 |
| master_99213 | chr14 | 67303844 | 67304144 | TTS | 10717 | NM_001037931 |
| | | | | (NM_001037931) | | |
| master_242668 | chr5 | 1.32E+08 | 1.32E+08 | intron | −98864 | NR_040704 |
| | | | | (NM_177047, | | |
| | | | | intron 4 of 18) | | |
| master_322307 | chrX | 1.65E+08 | 1.65E+08 | intron | 18690 | NM_183427 |
| | | | | (NM_183427, | | |
| | | | | intron 2 of 8) | | |
| master_86830 | chr13 | 89607351 | 89607651 | intron | 66865 | NM_013500 |
| | | | | (NM_013500, | | |
| | | | | intron 4 of 4) | | |
| master_213861 | chr4 | 71303472 | 71303898 | Intergenic | −768757 | NM_172694 |
| master_202348 | chr3 | 1.4E+08 | 1.4E+08 | Intergenic | −129863 | NR_105798 |
| master_167422 | chr2 | 62878811 | 62879111 | intron | 214634 | NM_145523 |
| | | | | (NM_133207, | | |
| | | | | intron 2 of 15) | | |
| master_234232 | chr5 | 71970289 | 71970763 | intron | 197657 | NM_178599 |
| | | | | (NM_008069, | | |
| | | | | intron 4 of 8) | | |
| master_187665 | chr3 | 35232412 | 35232980 | Intergenic | −310045 | NR_105799 |
| master_234187 | chr5 | 71440193 | 71440596 | Intergenic | 107811 | NM_030052 |
| master_259350 | chr6 | 1.1E+08 | 1.1E+08 | Intergenic | −830462 | NM_177328 |
| master_258496 | chr6 | 1.02E+08 | 1.02E+08 | Intergenic | −180230 | NR_027989 |
| master_48173 | chr11 | 40573086 | 40574312 | Intergenic | 118983 | NM_134017 |
| master_321811 | chrX | 1.59E+08 | 1.59E+08 | Intergenic | −417997 | NM_148945 |
| master_206682 | chr4 | 13098879 | 13099219 | Intergenic | 192212 | NM_001171801 |
| master_18228 | chr1 | 1.4E+08 | 1.4E+08 | intron | 49374 | NM_001081027 |
| | | | | (NM_001081027, | | |
| | | | | intron 1 of 27) | | |
| master_13459 | chr1 | 1.05E+08 | 1.05E+08 | Intergenic | 238544 | NM_178779 |
| master_34001 | chr10 | 63203062 | 63203362 | intron | 740 | NM_182992 |
| | | | | (NM_182992, | | |
| | | | | intron 1 of 19) | | |
| master_86097 | chr13 | 82847529 | 82847852 | Intergenic | −656344 | NM_001170537 |
| master_35633 | chr10 | 75475056 | 75475438 | Intergenic | 25737 | NR_045841 |
| master_202366 | chr3 | 1.4E+08 | 1.4E+08 | Intergenic | 59864 | NR_105798 |
| master_231620 | chr5 | 49337154 | 49337527 | Intergenic | −51681 | NM_001199244 |
| master_190653 | chr3 | 56362436 | 56362905 | Intergenic | −178969 | NM_030595 |
| master_40228 | chr10 | 1.09E+08 | 1.09E+08 | Intergenic | −221953 | NM_001252341 |
| master_309157 | chr9 | 1.01E+08 | 1.01E+08 | Intergenic | −9263 | NM_001100451 |
| master_214627 | chr4 | 81285558 | 81285911 | intron | 157071 | NM_010820 |
| | | | | (NM_010820, | | |
| | | | | intron 41 of 46) | | |
| master_235434 | chr5 | 79855800 | 79856100 | Intergenic | −340046 | NR_035474 |
| master_65261 | chr12 | 41254199 | 41254545 | intron | 230282 | NM_053122 |
| | | | | (NM_053122, | | |
| | | | | intron 4 of 6) | | |
| master_18681 | chr1 | 1.45E+08 | 1.45E+08 | Intergenic | −288830 | NM_022881 |
| master_189536 | chr3 | 49841996 | 49842296 | Intergenic | −84830 | NM_130448 |
| master_66244 | chr12 | 50842744 | 50843405 | Intergenic | −193851 | NM_008858 |
| master_202709 | chr3 | 1.43E+08 | 1.43E+08 | Intergenic | −80900 | NR_040551 |
| master_102414 | chr14 | 92829120 | 92829420 | Intergenic | 1059618 | NM_001271800 |
| master_233871 | chr5 | 67738361 | 67738661 | intron | 108920 | NM_001284345 |
| | | | | (NM_001284345, | | |
| | | | | intron 19 of 36) | | |
| master_24278 | chr1 | 1.86E+08 | 1.86E+08 | intron | 22566 | NR_030555 |
| | | | | (NR_030555, | | |
| | | | | intron 2 of 6) | | |
| master_125761 | chr16 | 55390415 | 55390776 | Intergenic | −107358 | NM_178720 |
| master_67853 | chr12 | 65490942 | 65491543 | Intergenic | 265725 | NM_027614 |
| master_143176 | chr18 | 19781226 | 19781526 | Intergenic | 220721 | NM_007882 |
| master_185782 | chr3 | 21025848 | 21026230 | Intergenic | −658862 | NM_175086 |
| master_6467 | chr1 | 53157918 | 53158218 | TTS | 29549 | NM_027070 |
| | | | | (NM_027070) | | |
| master_78555 | chr13 | 28269651 | 28269951 | Intergenic | 127317 | NM_023746 |
| master_206583 | chr4 | 12334826 | 12335403 | Intergenic | −163099 | NM_026558 |
| master_65848 | chr12 | 46766265 | 46766571 | intron | 52357 | NM_021361 |
| | | | | (NM_021361, | | |
| | | | | intron 2 of 4) | | |
| master_275834 | chr7 | 1.15E+08 | 1.15E+08 | Intergenic | 368207 | NR_040319 |
| master_47416 | chr11 | 34349345 | 34349680 | intron | 34690 | NM_001025382 |
| | | | | (NM_001025382, | | |
| | | | | intron 1 of 3) | | |
| master_80628 | chr13 | 42671542 | 42672340 | Intergenic | −8682 | NM_198419 |
| master_268122 | chr7 | 36257068 | 36257479 | Intergenic | −440845 | NM_172298 |
| master_232451 | chr5 | 55885696 | 55886323 | Intergenic | 1536017 | NR_038045 |
| master_306022 | chr9 | 75171597 | 75172088 | intron | −60172 | NM_001081322 |
| | | | | (NM_010864, | | |
| | | | | intron 21 of 40) | | |
| master_286193 | chr8 | 54864722 | 54865065 | intron | 85459 | NM_001253754 |
| | | | | (NM_001253754, | | |
| | | | | intron 1 of 6) | | |
| master_81259 | chr13 | 46092818 | 46093223 | Intergenic | −128029 | NM_009124 |
| master_84880 | chr13 | 73009221 | 73009521 | Intergenic | −192608 | NR_046196 |
| master_68953 | chr12 | 74265005 | 74265305 | intron | −19121 | NR_030734 |
| | | | | (NM_172804, | | |
| | | | | intron 6 of 6) | | |
| master_221646 | chr4 | 1.33E+08 | 1.33E+08 | intron | 11338 | NM_153423 |
| | | | | (NM_153423, | | |
| | | | | intron 1 of 8) | | |
| master_278746 | chr7 | 1.37E+08 | 1.37E+08 | intron | 110964 | NM_008598 |
| | | | | (NM_008598, | | |
| | | | | intron 2 of 4) | | |
| master_172511 | chr2 | 1.04E+08 | 1.04E+08 | intron | 45070 | NM_178890 |
| | | | | (NM_178890, | | |
| | | | | intron 1 of 16) | | |
| master_55235 | chr11 | 89767078 | 89767378 | Intergenic | −228673 | NM_001080933 |
| master_45219 | chr11 | 17625719 | 17626019 | Intergenic | 328006 | NM_026576 |
| master_158954 | chr19 | 54465474 | 54466001 | Intergenic | 420555 | NM_007417 |
| master_126935 | chr16 | 68666918 | 68667387 | Intergenic | −1046244 | NM_178721 |
| master_187519 | chr3 | 34337876 | 34338176 | Intergenic | −222355 | NR_015580 |
| master_43348 | chr11 | 4336141 | 4336474 | Intergenic | 69511 | NM_001039537 |
| master_145175 | chr18 | 35070679 | 35071318 | Intergenic | −47914 | NM_009818 |
| master_175271 | chr2 | 1.25E+08 | 1.25E+08 | Intergenic | −70649 | NM_175034 |
| master_156705 | chr19 | 36516882 | 36517261 | Intergenic | −37568 | NM_001163471 |
| master_127572 | chr16 | 76728547 | 76728946 | Intergenic | 282135 | NR_040573 |
| master_246732 | chr6 | 12385621 | 12385921 | intron | −276191 | NR_003631 |
| | | | | (NM_001164805, | | |
| | | | | intron 11 of 26) | | |
| master_232151 | chr5 | 53402765 | 53403065 | Intergenic | 135809 | NM_001145433 |
| master_179281 | chr2 | 1.55E+08 | 1.55E+08 | exon | 19476 | NM_001242558 |
| | | | | (NM_001242558, | | |
| | | | | exon 6 of 13) | | |
| master_13360 | chr1 | 1.04E+08 | 1.04E+08 | Intergenic | −340865 | NM_011800 |
| master_247291 | chr6 | 17338854 | 17339232 | intron | 31403 | NM_001243064 |
| | | | | (NM_001243064, | | |
| | | | | intron 1 of 1) | | |
| master_207002 | chr4 | 15660677 | 15661211 | Intergenic | −220320 | NM_009788 |
| master_174514 | chr2 | 1.19E+08 | 1.19E+08 | intron | −10475 | NM_001081971 |
| | | | | (NM_177568, | | |
| | | | | intron 19 of 31) | | |
| master_275684 | chr7 | 1.14E+08 | 1.14E+08 | intron | 119595 | NM_145584 |
| | | | | (NM_145584, | | |
| | | | | intron 4 of 15) | | |
| master_299711 | chr9 | 28822118 | 28822715 | intron | 1031147 | NM_177906 |
| | | | | (NM_177906, | | |
| | | | | intron 4 of 7) | | |
| master_294361 | chr8 | 1.15E+08 | 1.15E+08 | intron | 331942 | NM_019573 |
| | | | | (NM_019573, | | |
| | | | | intron 8 of 8) | | |
| master_177384 | chr2 | 1.41E+08 | 1.41E+08 | intron | −554867 | NM_178382 |
| | | | | (NM_001013802, | | |
| | | | | intron 5 of 18) | | |
| master_110821 | chr15 | 41054659 | 41054959 | intron | −392673 | NM_001130166 |
| | | | | (NM_011766, | | |
| | | | | intron 5 of 7) | | |
| master_315773 | chrX | 53129886 | 53130186 | Intergenic | −15631 | NM_019538 |
| master_179180 | chr2 | 1.55E+08 | 1.55E+08 | exon | −31145 | NM_029305 |
| | | | | (NM_001285446, | | |
| | | | | exon 6 of 11) | | |
| master_264631 | chr6 | 1.44E+08 | 1.44E+08 | Intergenic | −12208 | NR_045732 |
| master_275370 | chr7 | 1.12E+08 | 1.12E+08 | Intergenic | −88252 | NM_173739 |
| master_124553 | chr16 | 43482199 | 43482572 | intron | −62189 | NR_046026 |
| | | | | (NM_181058, | | |
| | | | | intron 3 of 6) | | |
| master_154410 | chr19 | 15230185 | 15230497 | Intergenic | −632358 | NM_011600 |
| master_126567 | chr16 | 63287895 | 63288253 | Intergenic | 433740 | NM_011173 |
| master_93492 | chr14 | 23052850 | 23053552 | intron | 41370 | NR_033550 |
| | | | | (NR_033550, | | |
| | | | | intron 2 of 3) | | |
| master_85601 | chr13 | 78658264 | 78658661 | Intergenic | −459480 | NM_010151 |
| master_6993 | chr1 | 57490092 | 57490392 | Intergenic | −83568 | NM_001037742 |
| master_5506 | chr1 | 45340886 | 45341293 | intron | 29551 | NM_009930 |
| | | | | (NM_009930, | | |
| | | | | intron 39 of 50) | | |
| master_91716 | chr14 | 9329645 | 9330198 | Intergenic | −554174 | NR_030680 |
| master_237792 | chr5 | 1.01E+08 | 1.01E+08 | Intergenic | 136132 | NM_172715 |
| master_91673 | chr14 | 8961589 | 8961948 | Intergenic | 295378 | NR_045968 |
| master_25393 | chr1 | 1.92E+08 | 1.92E+08 | exon | −8997 | NM_011633 |
| | | | | (NM_001290280, | | |
| | | | | exon 11 of 11) | | |
| master_232007 | chr5 | 52812384 | 52812813 | intron | −21537 | NM_024213 |
| | | | | (NM_030185, | | |
| | | | | intron 8 of 11) | | |
| master_31297 | chr10 | 43124190 | 43124919 | intron | 49976 | NM_175407 |
| | | | | (NM_175407, | | |
| | | | | intron 4 of 6) | | |
| master_122123 | chr16 | 23890468 | 23890956 | exon | 132 | NM_009215 |
| | | | | (NM_009215, | | |
| | | | | exon 1 of 2) | | |
| master_85792 | chr13 | 80235196 | 80235580 | Intergenic | −648034 | NM_001042591 |
| master_225818 | chr5 | 4249174 | 4249590 | Intergenic | 57015 | NM_001042670 |
| master_210888 | chr4 | 48083083 | 48083383 | exon | 31985 | NM_015743 |
| | | | | (NM_015743, | | |
| | | | | exon 6 of 6) | | |
| master_105695 | chr14 | 1.2E+08 | 1.2E+08 | intron | 51175 | NR_045621 |
| | | | | (NM_015820, | | |
| | | | | intron 1 of 1) | | |
| master_184769 | chr3 | 12039885 | 12040185 | Intergenic | −195676 | NR_040751 |
| master_220929 | chr4 | 1.29E+08 | 1.29E+08 | Intergenic | −34039 | NM_001081098 |
| master_186184 | chr3 | 24938546 | 24938846 | Intergenic | 1214611 | NM_001163387 |
| master_73613 | chr12 | 1.09E+08 | 1.09E+08 | intron | −32742 | NM_001163394 |
| | | | | (NM_001043335, | | |
| | | | | intron 15 of 21) | | |
| master_213995 | chr4 | 72524182 | 72524490 | Intergenic | 323092 | NR_027923 |
| master_158523 | chr19 | 50005333 | 50005829 | Intergenic | 673065 | NM_001252501 |
| master_283209 | chr8 | 28739073 | 28739387 | intron | 480406 | NM_153135 |
| | | | | (NM_153135, | | |
| | | | | intron 8 of 17) | | |
| master_126555 | chr16 | 63179254 | 63179665 | Intergenic | 325125 | NM_011173 |
| master_268220 | chr7 | 37024126 | 37024915 | Intergenic | 326402 | NM_172298 |
| master_65790 | chr12 | 46020869 | 46021169 | Intergenic | 797756 | NM_021361 |
| master_30928 | chr10 | 40949604 | 40949944 | Intergenic | 66240 | NM_031877 |
| master_246867 | chr6 | 13476822 | 13477204 | Intergenic | 63676 | NR_038149 |
| master_286738 | chr8 | 60036768 | 60037068 | Intergenic | −469206 | NM_011834 |
| master_105206 | chr14 | 1.17E+08 | 1.17E+08 | intron | 236947 | NM_011821 |
| | | | | (NM_001079844, | | |
| | | | | intron 1 of 9) | | |
| master_86154 | chr13 | 83480409 | 83480756 | Intergenic | −23452 | NM_001170537 |
| master_314987 | chrX | 41216876 | 41217275 | Intergenic | −184226 | NM_016886 |
| master_247636 | chr6 | 19858366 | 19858666 | Intergenic | 303174 | NR_105789 |
| master_246720 | chr6 | 12336565 | 12336865 | intron | −227135 | NR_003631 |
| | | | | (NM_001164805, | | |
| | | | | intron 18 of 26) | | |
| master_248448 | chr6 | 26976687 | 26976987 | Intergenic | 959913 | NR_030420 |
| master_164666 | chr2 | 38143922 | 38144313 | intron | 143267 | NM_146122 |
| | | | | (NM_146122, | | |
| | | | | intron 3 of 21) | | |
| master_105108 | chr14 | 1.16E+08 | 1.16E+08 | intron | −523278 | NM_001079844 |
| | | | | (NM 175500, | | |
| | | | | intron 7 of 7) | | |
| master_71108 | chr12 | 89724007 | 89724450 | intron | −88255 | NM_001252074 |
| | | | | (NM_172544, | | |
| | | | | intron 14 of 19) | | |
| master_110079 | chr15 | 35540725 | 35541182 | intron | 119965 | NR_035527 |
| | | | | (NM_177151, | | |
| | | | | intron 19 of 61) | | |
| master_143084 | chr18 | 18899743 | 18900043 | Intergenic | 1102204 | NM_007882 |
| master_214396 | chr4 | 77861388 | 77861688 | intron | 350357 | NM_011211 |
| | | | | (NM_011211, | | |
| | | | | intron 2 of 39) | | |
| master_208620 | chr4 | 31297884 | 31298184 | Intergenic | 633438 | NR_040655 |
| master_215836 | chr4 | 89621275 | 89621662 | Intergenic | −66730 | NM_175647 |
| master_166748 | chr2 | 58240655 | 58241085 | Intergenic | −35909 | NR_040365 |
| master_167577 | chr2 | 64853888 | 64854283 | Intergenic | 168681 | NM_016719 |
| master_186145 | chr3 | 24463943 | 24464243 | Intergenic | 1689214 | NM_001163387 |
| master_67210 | chr12 | 58695469 | 58695991 | Intergenic | 316287 | NM_009147 |
| master_150387 | chr18 | 72949140 | 72949782 | Intergenic | −598392 | NM_007831 |
| master_169975 | chr2 | 83518526 | 83519006 | Intergenic | −125812 | NM_026934 |
| master_32774 | chr10 | 55444849 | 55445204 | Intergenic | −661891 | NM_001163833 |
| master_210035 | chr4 | 43182631 | 43183064 | exon | −84312 | NM_177195 |
| | | | | (NM_021468, | | |
| | | | | exon 11 of 40) | | |
| master_58417 | chr11 | 1.12E+08 | 1.12E+08 | Intergenic | 752130 | NM_008425 |
| master_9533 | chr1 | 75662704 | 75663034 | Intergenic | 116603 | NM_009208 |
| master_103842 | chr14 | 1.04E+08 | 1.04E+08 | Intergenic | −20969 | NM_001136061 |
| master_47853 | chr11 | 36484475 | 36485184 | intron | 459412 | NM_001290702 |
| | | | | (NM_001290702, | | |
| | | | | intron 2 of 27) | | |
| master_159141 | chr19 | 55774233 | 55774679 | intron | 32646 | NM_001142923 |
| | | | | (NM_001142920, | | |
| | | | | intron 4 of 11) | | |
| master_177107 | chr2 | 1.38E+08 | 1.38E+08 | Intergenic | 48257 | NM_145534 |
| master_99747 | chr14 | 71462517 | 71462910 | Intergenic | 211440 | NR_046076 |
| master_47920 | chr11 | 36940885 | 36941185 | intron | 3206 | NM_001290702 |
| | | | | (NM_001290702, | | |
| | | | | intron 1 of 27) | | |
| master_113590 | chr15 | 66559082 | 66559382 | intron | 1814 | NM_172514 |
| | | | | (NM_172514, | | |
| | | | | intron 2 of 9) | | |
| master_87341 | chr13 | 93058332 | 93059176 | intron | −14550 | NR_036451 |
| | | | | (NM_023821, | | |
| | | | | intron 9 of 12) | | |
| master_200551 | chr3 | 1.29E+08 | 1.29E+08 | Intergenic | −294029 | NR_045704 |
| master_137906 | chr17 | 69247467 | 69247767 | exon | 29589 | NR_045428 |
| | | | | (NM_013813, | | |
| | | | | exon 7 of 22) | | |
| master_89695 | chr13 | 1.08E+08 | 1.08E+08 | Intergenic | −163283 | NM_011056 |
| master_126468 | chr16 | 62441247 | 62441767 | Intergenic | 345209 | NM_178925 |
| master_258783 | chr6 | 1.05E+08 | 1.05E+08 | intron | 315754 | NM_017383 |
| | | | | (NM_017383, | | |
| | | | | intron 12 of 22) | | |
| master_177442 | chr2 | 1.42E+08 | 1.42E+08 | intron | 917828 | NM_001081133 |
| | | | | (NM 001013802, | | |
| | | | | intron 8 of 18) | | |
| master_143653 | chr18 | 23436461 | 23436761 | intron | 21202 | NM_001285811 |
| | | | | (NM 001285811, | | |
| | | | | intron 1 of 18) | | |
| master_211377 | chr4 | 52265188 | 52265563 | Intergenic | 173589 | NR_045175 |
| master_299656 | chr9 | 28327951 | 28328405 | intron | 536909 | NM_177906 |
| | | | | (NM_177906, | | |
| | | | | intron 1 of 7) | | |
| master_121703 | chr16 | 21282274 | 21282650 | non-coding | 14410 | NR_046162 |
| | | | | (NR_046162, | | |
| | | | | exon 2 of 5) | | |
| master_289020 | chr8 | 77292542 | 77293050 | intron | −224260 | NR_028125 |
| | | | | (NM_030113, | | |
| | | | | intron 19 of 22) | | |
| master_266603 | chr7 | 19612288 | 19612588 | intron | −7970 | NM_016680 |
| | | | | (NM_009046, | | |
| | | | | intron 8 of 10) | | |
| master_57320 | chr11 | 1.04E+08 | 1.04E+08 | intron | 47595 | NM_008740 |
| | | | | (NM_008740, | | |
| | | | | intron 8 of 20) | | |
| master_313130 | chrX | 9308927 | 9309227 | exon | 25313 | NM_029588 |
| | | | | (NM_023500, | | |
| | | | | exon 3 of 3) | | |
| master_68501 | chr12 | 70999393 | 70999717 | intron | 16268 | NR_045056 |
| | | | | (NR_045055, | | |
| | | | | intron 3 of 3) | | |
| master_126457 | chr16 | 62288956 | 62289426 | Intergenic | 497525 | NM_178925 |

| | Gene Name | Name | ATAC_Specificity | PESCA_Specificity |
|---|---|---|---|---|
| master_174202 | Tmco5 | GRE1 | 54.42356 | 2.04789 |
| master_101787 | Diap3 | GRE2 | 45.73314 | 1.614378 |
| master_100995 | Olfm4 | GRE3 | 43.92524 | 3.936133 |
| master_10184 | Mir6344 | GRE4 | 43.75069 | 4.439794 |
| master_194536 | Mab21l2 | GRE5 | 38.67608 | 1.197707 |
| master_206695 | Triqk | GRE6 | 37.27652 | 2.578589 |
| master_85259 | Mir682 | GRE7 | 35.46862 | 2.965952 |
| master_211290 | Cylc2 | GRE8 | 33.25238 | 1.998991 |
| master_142964 | 4930545E07Rik | GRE9 | 32.84404 | 2.940086 |
| master_63024 | Gm17746 | GRE10 | 32.66948 | 1.491584 |
| master_137958 | A330050F15Rik | GRE11 | 32.4357 | 3.815681 |
| master_111792 | Rad21 | GRE12 | 32.02736 | 8.349333 |
| master_101297 | Pcdh17 | GRE13 | 30.6278 | 3.557628 |
| master_101845 | Tdrd3 | GRE14 | 30.21946 | 3.994573 |
| master_232280 | Gm10440 | GRE15 | 28.64534 | 2.031232 |
| master_102497 | Pcdh9 | GRE17 | 27.59488 | 3.227034 |
| master_67193 | Clec14a | GRE16 | 27.59488 | 4.716805 |
| master_300352 | 7630403G23Rik | GRE18 | 26.60366 | 2.094394 |
| master_165379 | 1700019E08Rik | GRE19 | 26.19532 | 7.608419 |
| master_226748 | Sema3a | GRE20 | 26.19532 | 2.734884 |
| master_168633 | 8430437L04Rik | GRE21 | 26.02076 | 2.25101 |
| master_168094 | Nostrin | GRE22 | 25.78698 | 9.124598 |
| master_250505 | Olfr459 | GRE23 | 25.78698 | 2.973378 |
| master_169135 | Plekha3 | GRE24 | 24.50143 | 4.132428 |
| master_213877 | Tle1 | GRE25 | 24.38742 | 1.607428 |
| master_48723 | Gm12159 | GRE26 | 23.97908 | 2.748332 |
| master_125071 | Pvrl3 | GRE27 | 23.97908 | 0.996998 |
| master_284632 | 2810404M03Rik | GRE29 | 23.57074 | 2.579391 |
| master_126619 | Epha3 | GRE28 | 23.57074 | 3.336779 |
| master_13877 | D830032E09Rik | GRE30 | 23.1624 | 1.042263 |
| master_149404 | Alpk2 | GRE31 | 22.98786 | 2.321702 |
| master_17921 | Mir181a-1 | GRE32 | 21.79033 | 1.199547 |
| master_273562 | Tenm4 | GRE33 | 21.76284 | 1.171052 |
| master_1929 | Crisp4 | GRE34 | 21.76284 | 2.160536 |
| master_209025 | Mir8118 | GRE37 | 21.3545 | 2.149829 |
| master_48719 | Gm12159 | GRE35 | 21.3545 | 1.515981 |
| master_231206 | Ldb2 | GRE38 | 21.3545 | 2.853684 |
| master_206581 | Fam92a | GRE36 | 21.3545 | 1.915442 |
| master_145539 | Pcdhgb5 | GRE39 | 21.17996 | 1.796272 |
| master_234138 | Gabrg1 | GRE43 | 20.94616 | 2.70608 |
| master_29489 | Themis | GRE40 | 20.94616 | 1.954794 |
| master_32108 | Grik2 | GRE41 | 20.94616 | 2.063917 |
| master_213912 | Tle1 | GRE42 | 20.94616 | 2.284112 |
| master_203978 | Eltd1 | GRE44 | 20.22442 | 7.224984 |
| master_303924 | Neo1 | GRE45 | 19.62251 | 1.315558 |
| master_24617 | Esrrg | GRE46 | 19.5466 | 3.401349 |
| master_284637 | 2810404M03Rik | GRE47 | 19.5466 | 2.335916 |
| master_27246 | Zc2hc1b | GRE48 | 19.25842 | 4.373391 |
| master_214332 | Ptprd | GRE50 | 19.13826 | 2.939487 |
| master_107992 | Cdh9 | GRE49 | 19.13826 | 3.204406 |
| master_47913 | Tenm2 | GRE51 | 18.72992 | 2.179119 |
| master_201188 | Dkk2 | GRE54 | 18.72992 | 1.025187 |
| master_108055 | 4921515E04Rik | GRE53 | 18.72992 | 1.325807 |
| master_207201 | Mmp16 | GRE55 | 18.72992 | 1.932495 |
| master_107942 | Cdh9 | GRE52 | 18.72992 | 1.124406 |
| master_277752 | Gm4265 | GRE56 | 18.40637 | 2.465126 |
| master_5879 | Mir6350 | GRE57 | 18.36401 | 5.568431 |
| master_211266 | Cylc2 | GRE58 | 18.364 | 6.716803 |
| master_15237 | Tfcp2l1 | GRE59 | 18.14703 | 2.842816 |
| master_127446 | Samsn1 | GRE60 | 18.09993 | 0.901705 |
| master_207259 | Mmp16 | GRE63 | 17.33036 | 1.544626 |
| master_85915 | 9330111N05Rik | GRE61 | 17.33036 | 1.309903 |
| master_185216 | Ythdf3 | GRE62 | 17.33036 | 1.936168 |
| master_286157 | Spcs3 | GRE64 | 17.13158 | 3.969822 |
| master_211163 | Grin3a | GRE65 | 17.0414 | 3.965299 |
| master_247854 | Cped1 | GRE68 | 16.92202 | 1.531165 |
| master_67398 | Gm20063 | GRE66 | 16.92202 | 1.690199 |
| master_175066 | Sqrdl | GRE67 | 16.92202 | 3.031597 |
| master_316282 | Ldoc1 | GRE69 | 16.92202 | 3.894411 |
| master_39511 | Rassf9 | GRE70 | 16.51368 | 4.229043 |
| master_188695 | D3Ertd751e | GRE74 | 16.51368 | 1.199453 |
| master_166708 | Galnt5 | GRE73 | 16.51368 | 2.502856 |
| master_297161 | Casp12 | GRE77 | 16.51368 | 2.781741 |
| master_55384 | 4930405D11Rik | GRE72 | 16.51368 | 2.435881 |
| master_284777 | Zfp42 | GRE76 | 16.51368 | 1.347361 |
| master_271292 | Mctp2 | GRE75 | 16.51368 | 1.390241 |
| master_40675 | 1700010J16Rik | GRE71 | 16.51368 | 2.506238 |
| master_258714 | Chl1 | GRE78 | 16.51368 | 1.303797 |
| master_154372 | Tle4 | GRE79 | 16.4688 | 2.229144 |
| master_216325 | Caap1 | GRE80 | 16.45098 | 7.98716 |
| master_104183 | Trim52 | GRE81 | 16.41243 | 5.047027 |
| master_154310 | Olfr1494 | GRE82 | 16.33913 | 2.689691 |
| master_246461 | Nxph1 | GRE83 | 16.07045 | 5.249903 |
| master_180377 | Ptprtos | GRE84 | 15.93079 | 2.533423 |
| master_17924 | Mir181a-1 | GRE85 | 15.93079 | 1.130762 |
| master_216328 | Caap1 | GRE86 | 15.93079 | 1.953948 |
| master_127878 | Tmprss15 | GRE87 | 15.64428 | 1.679649 |
| master_31540 | Prdm1 | GRE88 | 15.52246 | 3.930135 |
| master_90969 | Emb | GRE89 | 15.52246 | 2.29439 |
| master_152861 | Cbln2 | GRE90 | 15.46207 | 1.367071 |
| master_83032 | Spock1 | GRE91 | 15.29221 | 4.999408 |
| master_315069 | Sh2d1a | GRE94 | 15.11412 | 2.108611 |
| master_177079 | Btbd3 | GRE93 | 15.11412 | 3.383857 |
| master_98792 | Hmbox1 | GRE92 | 15.11412 | 3.358701 |
| master_7522 | Pard3b | GRE95 | 14.7636 | 2.697757 |
| master_234051 | Kctd8 | GRE96 | 14.70578 | 2.194772 |
| master_253151 | Grid2 | GRE97 | 14.70578 | 1.270838 |
| master_3497 | Gm5415 | GRE98 | 14.70578 | 0.879922 |
| master_67191 | Clec14a | GRE99 | 14.70578 | 2.767234 |
| master_307584 | Nt5e | GRE100 | 14.46554 | 2.145091 |
| master_21944 | Nuf2 | GRE102 | 14.29744 | 1.490519 |
| master_18469 | B3galt2 | GRE101 | 14.29744 | 1.487266 |
| master_292268 | Gm15679 | GRE106 | 14.29744 | 2.354974 |
| master_258969 | Crbn | GRE103 | 14.29744 | 2.545115 |
| master_278030 | Cpxm2 | GRE104 | 14.29744 | 1.467424 |
| master_292154 | Mir28c | GRE105 | 14.29744 | 4.109301 |
| master_94135 | Ccdc66 | GRE107 | 14.0991 | 1.018433 |
| master_202401 | Mirlet7j | GRE108 | 13.75703 | 1.953057 |
| master_296959 | Mir1903 | GRE109 | 13.71455 | 2.007763 |
| master_242798 | Auts2 | GRE110 | 13.66486 | 2.781013 |
| master_100196 | Mir466f-3 | GRE111 | 13.61387 | 1.264524 |
| master_5836 | Mir6350 | GRE112 | 13.41737 | 5.20298 |
| master_283415 | Dusp26 | GRE113 | 13.34185 | 4.281619 |
| master_176631 | Hao1 | GRE116 | 13.30622 | 1.038939 |
| master_85927 | 9330111N05Rik | GRE115 | 13.30622 | 2.988921 |
| master_24377 | Lyplal1 | GRE114 | 13.30622 | 2.756189 |
| master_246625 | AA545190 | GRE117 | 13.30529 | 3.00865 |
| master_191318 | Arhgef26 | GRE118 | 13.22453 | 0.934956 |
| master_301220 | 3110039I08Rik | GRE120 | 13.04865 | 1.033078 |
| master_202524 | Bmpr1b | GRE119 | 13.04865 | 1.40396 |
| master_215096 | Bnc2 | GRE122 | 12.89788 | 1.045062 |
| master_307975 | Zic4 | GRE125 | 12.89788 | 2.929242 |
| master_293057 | Ranbp10 | GRE124 | 12.89788 | 1.053188 |
| master_289251 | Slc10a7 | GRE123 | 12.89788 | 2.699745 |
| master_156217 | 8430431K14Rik | GRE121 | 12.89788 | 2.489354 |
| master_122150 | Bcl6 | GRE126 | 12.88183 | 2.389765 |
| master_99213 | Gm6878 | GRE127 | 12.68908 | 2.026174 |
| master_242668 | 4930563F08Rik | GRE128 | 12.56359 | 2.274754 |
| master_322307 | Glra2 | GRE129 | 12.49534 | 1.198567 |
| master_86830 | Hapln1 | GRE131 | 12.48954 | 1.051039 |
| master_213861 | Megf9 | GRE136 | 12.48954 | 3.920748 |
| master_202348 | Mirlet7j | GRE134 | 12.48954 | 3.125495 |
| master_167422 | Gca | GRE132 | 12.48954 | 1.738808 |
| master_234232 | Commd8 | GRE138 | 12.48954 | 2.529683 |
| master_187665 | Mir6378 | GRE133 | 12.48954 | 1.028798 |
| master_234187 | Cox7b2 | GRE137 | 12.48954 | 2.815921 |
| master_259350 | Grm7 | GRE140 | 12.48954 | 1.25086 |
| master_258496 | Gm9871 | GRE139 | 12.48954 | 2.019164 |
| master_48173 | Mat2b | GRE130 | 12.48954 | 1.043617 |
| master_321811 | Rps6ka3 | GRE141 | 12.48954 | 1.091957 |
| master_206682 | Triqk | GRE135 | 12.48954 | 1.070723 |
| master_18228 | Kcnt2 | GRE143 | 12.48954 | 1.75152 |
| master_13459 | Rnf152 | GRE142 | 12.48954 | 2.999347 |
| master_34001 | Mypn | GRE144 | 12.48954 | 3.428242 |
| master_86097 | Mef2c | GRE145 | 12.48666 | 2.070103 |
| master_35633 | 4933407G14Rik | GRE146 | 12.32138 | 4.293949 |
| master_202366 | Mirlet7j | GRE150 | 12.0812 | 1.728403 |
| master_231620 | Kcnip4 | GRE152 | 12.0812 | 0.869382 |
| master_190653 | Nbea | GRE149 | 12.0812 | 2.754418 |
| master_40228 | Syt1 | GRE147 | 12.0812 | 3.062184 |
| master_309157 | Msl2 | GRE154 | 12.0812 | 1.324496 |
| master_214627 | Mpdz | GRE151 | 12.0812 | 3.933542 |
| master_235434 | Mir669m-1 | GRE153 | 12.0812 | 1.114397 |
| master_65261 | Immp2l | GRE148 | 12.0812 | 1.776374 |
| master_18681 | Rgs18 | GRE155 | 12.0812 | 2.363578 |
| master_189536 | Pcdh18 | GRE156 | 11.98507 | 4.685037 |
| master_66244 | Prkd1 | GRE157 | 11.90044 | 2.06013 |
| master_202709 | A830019L24Rik | GRE158 | 11.87653 | 2.569771 |
| master_102414 | Pcdh9 | GRE159 | 11.72941 | 2.998667 |
| master_233871 | Atp8a1 | GRE160 | 11.71329 | 1.32488 |
| master_24278 | Mir297c | GRE161 | 11.70704 | 2.088993 |
| master_125761 | Zpld1 | GRE162 | 11.65123 | 1.217694 |
| master_67853 | Wdr20rt | GRE163 | 11.60678 | 9.486783 |
| master_143176 | Dsc3 | GRE164 | 11.50643 | 1.092974 |
| master_185782 | Agtr1b | GRE165 | 11.50643 | 0.946687 |
| master_6467 | 1700019A02Rik | GRE166 | 11.50643 | 1.371648 |
| master_78555 | Prl5a1 | GRE167 | 11.35372 | 3.666029 |
| master_206583 | Fam92a | GRE168 | 11.34256 | 3.093637 |
| master_65848 | Nova1 | GRE169 | 11.31423 | 1.173537 |
| master_275834 | A730082K24Rik | GRE170 | 11.2959 | 3.401681 |
| master_47416 | Fam196b | GRE171 | 11.23479 | 3.682435 |
| master_80628 | Phactr1 | GRE172 | 11.19301 | 1.223931 |
| master_268122 | Tshz3 | GRE173 | 11.16998 | 2.079197 |
| master_232451 | Gm10440 | GRE174 | 11.14319 | 1.335985 |
| master_306022 | Myo5c | GRE175 | 11.14319 | 1.093775 |
| master_286193 | Gpm6a | GRE176 | 11.13129 | 1.062735 |
| master_81259 | Atxn1 | GRE177 | 11.09708 | 1.036437 |
| master_84880 | D730050B12Rik | GRE179 | 11.08998 | 3.76879 |
| master_68953 | 1700086L19Rik | GRE178 | 11.08998 | 3.35081 |
| master_221646 | Wasf2 | GRE180 | 11.08998 | 1.83183 |
| master_278746 | Mgmt | GRE181 | 11.08998 | 1.663423 |
| master_172511 | Abtb2 | GRE182 | 11.05082 | 5.071098 |
| master_55235 | Ankfn1 | GRE183 | 10.90312 | 2.087424 |
| master_45219 | Etaa1 | GRE184 | 10.85562 | 5.179615 |
| master_158954 | Adra2a | GRE186 | 10.80111 | 2.824125 |
| master_126935 | Cadm2 | GRE185 | 10.80111 | 2.86257 |
| master_187519 | Sox2ot | GRE187 | 10.73751 | 3.18378 |
| master_43348 | Lif | GRE188 | 10.68164 | 4.383432 |
| master_145175 | Ctnna1 | GRE190 | 10.68164 | 1.483908 |
| master_175271 | Slc24a5 | GRE192 | 10.68164 | 2.532015 |
| master_156705 | Hectd2 | GRE191 | 10.68164 | 2.687629 |
| master_127572 | 1700041M19Rik | GRE189 | 10.68164 | 2.680087 |
| master_246732 | Gm6578 | GRE195 | 10.68164 | 0.888242 |
| master_232151 | Smim20 | GRE194 | 10.68164 | 1.128666 |
| master_179281 | Ncoa6 | GRE193 | 10.68164 | 1.138425 |
| master_13360 | Cdh20 | GRE196 | 10.68164 | 1.930547 |
| master_247291 | Cav1 | GRE197 | 10.67835 | 3.862841 |
| master_207002 | Calb1 | GRE198 | 10.51752 | 1.292931 |
| master_174514 | Ankrd63 | GRE199 | 10.45144 | 1.58715 |
| master_275684 | Spon1 | GRE200 | 10.27751 | 3.738082 |
| master_299711 | Opcml | GRE212 | 10.2733 | 0.804788 |
| master_294361 | Wwox | GRE211 | 10.2733 | 1.182984 |
| master_177384 | Flrt3 | GRE207 | 10.2733 | 2.094105 |
| master_110821 | Oxr1 | GRE203 | 10.2733 | 1.332164 |
| master_315773 | Plac1 | GRE213 | 10.2733 | 1.185207 |
| master_179180 | 1700003F12Rik | GRE208 | 10.2733 | 1.246752 |
| master_264631 | 1700060C16Rik | GRE209 | 10.2733 | 3.233375 |
| master_275370 | Galnt18 | GRE210 | 10.2733 | 1.602827 |
| master_124553 | Gm15713 | GRE204 | 10.2733 | 1.659611 |
| master_154410 | Tle4 | GRE206 | 10.2733 | 1.257922 |
| master_126567 | Pros1 | GRE205 | 10.2733 | 4.821853 |
| master_93492 | Gm10248 | GRE202 | 10.2733 | 3.740895 |
| master_85601 | Nr2f1 | GRE201 | 10.2733 | 2.055457 |
| master_6993 | Tyw5 | GRE215 | 10.2733 | 2.898172 |
| master_5506 | Col3a1 | GRE214 | 10.2733 | 3.16526 |
| master_91716 | Mnd1-ps | GRE216 | 10.2733 | 2.966396 |
| master_237792 | Agpat9 | GRE217 | 10.25055 | 1.906829 |
| master_91673 | 4930455B14Rik | GRE218 | 10.20506 | 2.719016 |
| master_25393 | Traf5 | GRE219 | 10.18832 | 3.113856 |
| master_232007 | Anapc4 | GRE220 | 10.13754 | 1.984117 |
| master_31297 | Sobp | GRE221 | 10.07523 | 0.699328 |
| master_122123 | Sst | GRE222 | 10.06429 | 1.322329 |
| master_85792 | Arrdc3 | GRE223 | 10.04386 | 0.942088 |
| master_225818 | Mterf1b | GRE224 | 9.941508 | 1.023699 |
| master_210888 | Nr4a3 | GRE225 | 9.893021 | 3.811214 |
| master_105695 | 1700006F04Rik | GRE226 | 9.891833 | 1.103087 |
| master_184769 | Gm10745 | GRE227 | 9.872822 | 1.35767 |
| master_220929 | Zfp362 | GRE245 | 9.864962 | 1.978169 |
| master_186184 | Nlgn1 | GRE240 | 9.864962 | 2.632975 |
| master_73613 | Evl | GRE231 | 9.864962 | 1.169432 |
| master_213995 | C630043F03Rik | GRE242 | 9.864962 | 2.95172 |
| master_158523 | Sorcs1 | GRE238 | 9.864962 | 1.495408 |
| master_283209 | Unc5d | GRE251 | 9.864962 | 1.214519 |
| master_126555 | Pros1 | GRE236 | 9.864962 | 1.635882 |
| master_268220 | Tshz3 | GRE250 | 9.864962 | 1.131852 |
| master_65790 | Nova1 | GRE229 | 9.864962 | 2.186033 |
| master_30928 | Wasf1 | GRE228 | 9.864962 | 1.133757 |
| master_246867 | 1700016P04Rik | GRE247 | 9.864962 | 3.094225 |
| master_286738 | Aadat | GRE252 | 9.864962 | 1.240162 |
| master_105206 | Gpc6 | GRE234 | 9.864962 | 2.659111 |
| master_86154 | Mef2c | GRE232 | 9.864962 | 1.063772 |
| master_314987 | Gria3 | GRE253 | 9.864962 | 3.181121 |
| master_247636 | Mir6370 | GRE248 | 9.864962 | 1.089155 |
| master_246720 | Gm6578 | GRE246 | 9.864962 | 0.883154 |
| master_248448 | Mir592 | GRE249 | 9.864962 | 3.641407 |
| master_164666 | Dennd1a | GRE239 | 9.864962 | 1.18446 |
| master_105108 | Gpc6 | GRE233 | 9.864962 | 1.793384 |
| master_71108 | Nrxn3 | GRE230 | 9.864962 | 1.383043 |
| master_110079 | Mir599 | GRE235 | 9.864962 | 1.266069 |
| master_143084 | Dsc3 | GRE237 | 9.864962 | 2.189808 |
| master_214396 | Ptprd | GRE243 | 9.864962 | 2.920626 |
| master_208620 | 4930556G01Rik | GRE241 | 9.864962 | 1.678081 |
| master_215836 | Dmrta1 | GRE244 | 9.864962 | 1.450679 |
| master_166748 | Gm13544 | GRE255 | 9.86496 | 0.927592 |
| master_167577 | Grb14 | GRE256 | 9.86496 | 3.197471 |
| master_186145 | Nlgn1 | GRE257 | 9.86496 | 2.301026 |
| master_67210 | Sec23a | GRE254 | 9.86496 | 1.716511 |
| master_150387 | Dcc | GRE258 | 9.824919 | 1.076888 |
| master_169975 | Zc3h15 | GRE259 | 9.701785 | 1.577381 |
| master_32774 | Msl3l2 | GRE260 | 9.657466 | 0.975373 |
| master_210035 | Atp8b5 | GRE261 | 9.632925 | 2.42893 |
| master_58417 | Kcnj2 | GRE262 | 9.626664 | 2.713361 |
| master_9533 | Slc4a3 | GRE263 | 9.601577 | 2.679371 |
| master_103842 | Ednrb | GRE264 | 9.581933 | 1.312873 |
| master_47853 | Tenm2 | GRE265 | 9.48924 | 1.109632 |
| master_159141 | Tcf7l2 | GRE266 | 9.423469 | 2.409842 |
| master_177107 | Btbd3 | GRE267 | 9.413072 | 1.56881 |
| master_99747 | Gm4251 | GRE268 | 9.385473 | 2.476358 |
| master_47920 | Tenm2 | GRE269 | 9.346406 | 2.993215 |
| master_113590 | Tmem71 | GRE270 | 9.346406 | 0.877458 |
| master_87341 | Gm4814 | GRE271 | 9.324092 | 2.881709 |
| master_200551 | D030025E07Rik | GRE273 | 9.230887 | 2.130008 |
| master_137906 | 2410021H03Rik | GRE272 | 9.230887 | 2.462781 |
| master_89695 | Pde4d | GRE274 | 9.222359 | 3.076912 |
| master_126468 | Nsun3 | GRE275 | 9.164146 | 1.129087 |
| master_258783 | Cntn6 | GRE276 | 9.158559 | 3.462615 |
| master_177442 | Kif16b | GRE277 | 9.115566 | 1.133672 |
| master_143653 | Dtna | GRE278 | 9.03805 | 1.360641 |
| master_211377 | Smc2os | GRE279 | 9.030095 | 0.931531 |
| master_299656 | Opcml | GRE280 | 8.904109 | 1.352754 |
| master_121703 | Gm16863 | GRE281 | 8.876421 | 1.327734 |
| master_289020 | 0610038B21Rik | GRE282 | 8.874542 | 1.196905 |
| master_266603 | Clasrp | GRE286 | 8.873734 | 2.271913 |
| master_57320 | Nsf | GRE283 | 8.873734 | 2.532934 |
| master_313130 | 1700012L04Rik | GRE287 | 8.873734 | 1.065551 |
| master_68501 | 3110056K07Rik | GRE284 | 8.873734 | 3.060029 |
| master_126457 | Nsun3 | GRE285 | 8.873734 | 2.748813 |

1. An adeno-associated virus (AAV) vector, comprising:
a. at least one inverted terminal repeat;
b. at least one gene regulatory element (GRE);
c. an expression cassette; and
d. a polyadenylation tail.

2. The AAV vector of claim 1, wherein the at least one GRE exhibits cell-type specificity.

3. The AAV vector of claim 1, wherein the at least one GRE is selected from the group consisting of: GRE12, GRE19, GRE22, GRE44, and GRE80.

4. The AAV vector of claim 1, wherein the AAV is selected from the group consisting of: bovine AAV (b-AAV); canine AAV (CAAV); mouse AAV1; caprine AAV; rat AAV; avian AAV (AAAV); AAV1; AAV2; AAV3b; AAV4; AAV5; AAV6; AAV7; AAV8; AAV9; AAV10; AAV11; AAV12; and AAV13.

5. The AAV vector of claim 1, wherein the AAV vector encodes an AAV capsid without a functional Rep protein.

6. The AAV vector of claim 1, wherein the AAV vector encodes an AAV capsid without one or more of VP1, VP2 and VP3.

7. A host cell comprising the AAV vector of claim 1.

8. A method of screening for adeno-associated virus (AAV) cell-type specific gene regulatory elements (GREs), comprising:
a. labeling a library of GREs with barcodes comprising a nucleic acid, wherein each of the barcodes is associated with a GRE structure, function, or both, in the library of GREs;
b. packaging the library of labeled GREs into AAV to generate an AAV library;
c. administering the AAV library to an organism;
d. detecting the barcodes in one or more cell types in the organism; and
e. identifying the GRE based on the cell type of interest and detected barcodes, thereby screening cell-type specific GREs.

9. The method of claim 8, wherein labeling the library of GREs comprises amplifying GREs using polymerase chain reaction (PCR) with a primer comprising a vector cloning site and a barcode sequence.

10. The method of claim 9, wherein the barcode sequence is about 7-15 base pairs.

11. The method of claim 10, wherein the barcode is 10 base pairs.

12. The method of claim 8, wherein packaging the library of labeled GREs into the AAV library comprises shuttling of the GRE PCR products into an AAV vector.

13. The method of claim 8, wherein detecting the barcodes in one or more cell types in the organism comprises single cell RNA sequencing (sc-RNA seq) or single nucleus RNA sequencing (sn-RNA seq).

14. The method of claim 8, wherein detecting the barcodes in single cells in the organism comprises single cell RNA sequencing (sc-RNA seq).

15. The method of claim 8, wherein each of the barcodes is unique to a GRE in the library of GREs.

16. The method of claim 13, wherein detecting the barcodes in one or more cell types in the organism comprises enrichment of RNA transcripts.

17. The method of claim 16, wherein enrichment of RNA transcripts comprises reverse transcribing RNA transcripts to generate complementary DNA (cDNA), amplifying the cDNA using second strand synthesis, and transcription of the cDNA to generate RNA intermediates.

18. The method of claim 17, wherein the RNA intermediates are amplified using PCR.

19. The method of claim 8, wherein detecting the barcodes in one or more cell types in the organism comprises capturing nuclei of the one or more cell types in hydrogels comprising cell barcode single primers.

20. A composition, comprising a nucleic acid sequence at least 70%, 80%, 85%, 90%, 95%, 96%, 97%, 98%, 99% or 100% identical to one of sequence GRE12, GRE19, GRE22, GRE44 or GRE80.
